(19) United States
(12) Patent Application Publication
(10) Pub. No.: US 2023/0399608 A1
(43) Pub. Date: Dec. 14, 2023

(54) PROCESS FOR ESTABLISHING A HUMAN TESTICULAR TISSUE CULTURE SYSTEM

(71) Applicant: PATERNA BIOSCIENCES INC., Salt Lake City, UT (US)

(72) Inventors: Jim M. Hotaling, Morristown, NJ (US); Brad R. Cairns, Morristown, NJ (US); Jingtao Guo, Morristown, NJ (US); Xichen Nie, Morristown, NJ (US)

(73) Assignee: PATERNA BIOSCIENCES INC., Salt Lake City, UT (US)

(21) Appl. No.: 18/346,691

(22) Filed: Jul. 3, 2023

Related U.S. Application Data
(63) Continuation-in-part of application No. PCT/US22/11179, filed on Jan. 4, 2022.
(60) Provisional application No. 63/133,633, filed on Jan. 4, 2021.

(51) Int. Cl. C12N 5/076; C12Q 1/6888

(52) U.S. Cl. CPC C12N 5/061; C12Q 1/6888; C12N 2501/392; C12N 2501/115; C12N 2501/13; C12N 2501/60

(57) ABSTRACT

The present disclosure provides an iterative process for identifying culture conditions that maintain identity, growth, and survival of testicular cells in vitro. Testicular cell culturing systems for supporting human spermatogenesis and culture using identified culture conditions are also provided. The methods and the culture conditions can be used to culture healthy and viable spermatozoa with lower rates of deleterious or de novo mutations or epigenetic perturbations from fertile and infertile men for future use with assisted reproductive technologies.

### CROSS-REFERENCE TO RELATED APPLICATIONS

The present application is a continuation-in-part of International Application number PCT/US2022/011179, filed on Jan. 4, 2022, which claims the benefit of U.S. Provisional Application No. 63/133,633, filed Jan. 4, 2021, the entire contents of each of the aforementioned applications is hereby incorporated by reference.

### FIELD OF THE INVENTION

The present disclosure provides an iterative process for identifying culture conditions that support human testicular germ cell proliferation in vitro and testicular tissue and germ cell cultures supportive of germ cell proliferation while maintaining identity, growth, and survival of the testicular germ cells.

### BACKGROUND OF THE INVENTION

A key need is the ability to culture human germ cells long term, at a scale needed for analysis at a transcriptome-scale manner, and in a manner that fully preserves their identity and functionality for spermatogenesis. However, the field of human male fertility is impeded by the lack of tools for studying spermatogenesis. There are no current successful ways of accomplishing these needs.

Through a wide range of approaches, considerable progress in understanding gametogenesis and germline-niche communication has been achieved in mice. In addition, spermatogonia have been successfully cultured, and methods developed to produce functional sperm from cultured spermatogonia that are capable of successful in vitro fertilization and generation of viable and fertile mice. In contrast, in humans, although adult testis physiology is well described, less is known about spermatogonial stem cells (SSCs), proliferative spermatogonia and their regulation, and long-term culturing of human spermatogonia coupled to genomics approaches to ensure their identity has not been achieved. Accordingly, methods and systems for in vitro culture of testicular germline cells (spermatogonia) are needed, which are the predecessors of spermatogenesis. To accomplish, a new approach is needed.

### SUMMARY OF THE INVENTION

One aspect of the instant disclosure encompasses an iterative process for identifying culture conditions supportive of testicular germ cell proliferation in vitro. The process comprises identifying one or more dysregulated pathways in testicular cells cultured in a first set of culture conditions by (1) culturing testicular tissue or isolated testicular germ cells in vitro in a first culture medium, wherein the testicular tissue comprises seminiferous tubules and testicular germ cells, and wherein the first culture medium supports a first level of proliferation of germ cells; (2) profiling transcriptomes of single testicular cells obtained from the testicular tissue or isolated testicular germ cells; and (3) identifying RNA transcripts differentially expressed in cultured tissue or germ cells when compared to RNA transcripts expressed in cultured tissue or germ cells of corresponding cell types obtained from control testicular tissue or isolated testicular germ cells, wherein differentially expressed RNA transcripts identify one or more dysregulated biological pathways in cells of the testicular tissue or isolated testicular germ cells cultured in the first set of culture conditions.

The process then comprises identifying one or more proliferation factors that improve the level of proliferation of testicular germ cells by: (1) culturing testicular tissue or isolated testicular germ cells in vitro in one or more second culture media, wherein the one or more second culture media comprise the first culture medium supplemented with one or more factors that regulate a biological pathway identified in (a); and (2) identifying one or more second culture media that support improved levels of germ cell proliferation when compared to the first level of germ cell proliferation in the first culture medium, thereby identifying the one or more factors that improve the level of proliferation of testicular germ cells.

The process can be repeated iteratively to identify additional factors that improve the level of proliferation of testicular germ cells. The culture conditions that support testicular germ cell proliferation comprise culture media supplemented with one or more factors identified in the process or the iterated processes.

Another aspect of the instant disclosure encompasses a culture medium supportive of testicular germ cell proliferation in vitro, the culture medium comprising a base medium supplemented with proliferation factors that improve the level of proliferation of testicular germ cells in vitro. The culture medium can be a medium identified using a process described above.

In some aspects, the culture medium comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, Testosterone at a concentration ranging from about 9 uM to about 11 uM, and Echinomycin at a concentration ranging from about 4 ng/ml to about 6 ng/ml. (Condition 2) In other aspects, the culture medium comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, and Testosterone at a concentration ranging from about 9 uM to about 11 uM. (C2) In yet other aspects, the culture medium comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, and Testosterone at a concentration ranging from about 78 uM to about 82 uM. (C2 with eight times testosterone)

An additional aspect of the instant disclosure encompasses a testicular cell culture for culturing testicular germ cells in vitro. The cell culture comprises testicular tissue comprising germ cells or testicular germ cells; and a culture medium comprising base media, and one or more factors that improve the level of proliferation of testicular germ cells in vitro.

In some aspects, the testicular tissue comprises seminiferous tubules comprising the testicular germ cells and the culture medium comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, Testosterone at a concentration ranging from about 9 uM to about 11 uM, and Echinomycin at a concentration ranging from about 4 ng/ml to about 6 ng/ml.

In some aspects, the testicular tissue comprises seminiferous tubules comprising the testicular germ cells and the culture medium comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, Testosterone at a concentration ranging from about 9 uM to about 11 uM, and Echinomycin at a concentration ranging from about 4 ng/ml to about 6 ng/ml.

In yet other aspects, the testicular germ cells comprise SSC, developing spermatogonia, or a combination thereof and wherein the culture medium comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, and Testosterone at a concentration ranging from about 78 uM to about 82 uM. (C2 with eight times testosterone)

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein.

FIG. 1A depicts a single-cell transcriptome profiling and analysis of the human fetal and postnatal testis. Dimension reduction presentation (via UMAP) of combined single-cell transcriptome data from embryonic, fetal, and infant human testes (n=30,045). Each dot represents a single cell and is colored according to its age/donor of origin. For each cell cluster, 1 cell marker is shown in the main figure, accompanied by a gallery of additional markers in FIG. 8. See also FIGS. 7A-7C and FIG. 8.

FIG. 1B depicts a single-cell transcriptome profiling and analysis of the human fetal and postnatal testis. Dimension reduction presentation (via UMAP) of combined single-cell transcriptome data from embryonic, fetal, and infant human testes (n=30,045). Each dot represents a single cell and is colored according to its age/donor of origin. For each cell cluster, 1 cell marker is shown in the main figure, accompanied by a gallery of additional markers in FIG. 8. See also FIGS. 7A-7C and FIG. 8.

FIG. 1C depicts a single-cell transcriptome profiling and analysis of the human fetal and postnatal testis. Expression patterns of selected markers (genes) are projected on the UMAP plot (FIG. 1A). For each cell cluster, 1 cell marker is shown in the main figure, accompanied by a gallery of additional markers in FIG. 8. See also FIGS. 7A-7C and FIG. 8.

FIG. 2A depicts gene expression dynamics during the development of human PGCs to adult spermatogonia. Focused analysis (t-SNE and pseudotime) of the profiled germ cells (cluster 12 from FIG. 1B) combined with infant germ cells and adult spermatogonia states (from Guo et al., 2018) revealed a single pseudo-developmental trajectory for germ cell development from embryo to adult. Cells are colored based on the ages of the donors. Differential gene expression levels use a Z score as defined by the color key; associated GO terms (using DAVID version 6.7) are given on the right of the corresponding gene clusters. See also FIG. 9 and FIG. 10.

FIG. 2B depicts gene expression dynamics during the development of human PGCs to adult spermatogonia. Expression patterns of known PGC and germ cell markers projected onto the tSNE plot from (FIG. 2A). See also FIG. 9 and FIG. 10.

FIG. 2C depicts gene expression dynamics during the development of human PGCs to adult spermatogonia. k-means clustering of genes exhibiting differential expression (n=2,448) along the germ cell pseudo-developmental trajectory. Each row represents a gene, and each column represents a single cell, with columns/cells placed in the pseudotime order defined in (FIG. 2A). Differential gene expression levels use a Z score as defined by the color key; associated GO terms (using DAVID version 6.7) are given on the right of the corresponding gene clusters. See also FIG. 9 and FIG. 10.

FIG. 2D depicts a gene expression dynamics during the development of human PGCs to adult spermatogonia. Protein co-immunofluorescence for markers of proliferation (MKI67), pluripotency (NANOG), and germ cells (DDX4) in samples from 5 to 19 weeks, and their corresponding quantification. See also FIG. 9 and FIG. 10.

FIG. 2E depicts gene expression dynamics during the development of human PGCs to adult spermatogonia. Protein co-immunofluorescence for germ cell (DDX4) and state 0 (PIWIL4) markers in samples from 8 to 17 weeks. See also FIG. 9 and FIG. 10.

FIG. 2F depicts gene expression dynamics during the development of human PGCs to adult spermatogonia. Quantification of the proportion of PIWIL4+ germ cells (DDX4+) in weeks 12-16 fetal testis samples. At least 100 cells per replicate and 3 replicates were counted. Each replicate was from a unique donor. Data show the means±SEMs (1-way ANOVA followed by a Tukey's post-test). Adjusted *p=0.0136, **p=0.0048, and ***p % 0.0008. See also FIG. 9 and FIG. 10.

FIG. 3A depicts the specification of interstitial and Sertoli lineages. Focused analysis (UMAP and pseudotime) of the testicular niche cells (clusters 1-11 from FIG. 1B), with cells colored according to the ages of the donors. See also FIG. 11.

FIG. 3B depicts the specification of interstitial and Sertoli lineages. Deconvolution of the plot in (FIG. 3A) according to the ages of the donors. See also FIG. 11.

FIG. 3C depicts the specification of interstitial and Sertoli lineages Focused analysis (in FIG. 3A) of the testicular niche cells (clusters 1-11 from FIG. 1B), with cells colored according to the ages/donors of origin. See also FIG. 11.

FIG. 3D depicts the specification of interstitial and Sertoli lineages. Expression patterns of known progenitor, interstitial/Leydig, and Sertoli markers (genes) projected onto the plot from (FIG. 3A). See also FIG. 11.

FIG. 4A depicts the gene expression dynamics during specification of interstitial and Sertoli lineages. k-means clustering of genes exhibiting differential expression (n=1,578) along interstitial/Leydig and Sertoli specification. Each row represents a gene, and each column represents a single cell, with columns/cells placed in the pseudotime order defined in FIG. 3A. Differential gene expression levels use a Z score, as defined by the color key; associated GO terms (using DAVID version 6.7) are given on the right of the corresponding gene clusters. See also FIG. 12.

FIG. 4B depicts the gene expression dynamics during specification of interstitial and Sertoli lineages. Immunostaining of Leydig marker CYP17A1 (cyan) in samples from 5 to 16 weeks. See also FIG. 12.

FIG. 4C depicts the gene expression dynamics during specification of interstitial and Sertoli lineages. Analysis to reveal differentially expressed genes during Leydig cell differentiation from fetal to infant stages. Violin plot on the left of each panel displays the fold change (x axis) and adjusted p value (y axis). The right part of each panel represents the enriched GO terms and the associated p value. See also FIG. 12.

FIG. 4D depicts the gene expression dynamics during specification of interstitial and Sertoli lineages. Analysis to reveal differentially expressed genes during Sertoli cell differentiation from fetal to infant stages. Violin plot on the left of each panel displays the fold change (x axis) and adjusted p value (y axis). The right part of each panel represents the enriched GO terms and the associated p value. See also FIG. 12.

FIG. 4E depicts the gene expression dynamics during specification of interstitial and Sertoli lineages. Immunostaining of Leydig marker CYP17A1 (cyan) in fetal and postnatal testis samples. See also FIG. 12.

FIG. 4F depicts the gene expression dynamics during specification of interstitial and Sertoli lineages. Pseudotime trajectory (combined Monocle analysis) of fetal interstitial cells, prepubertal Leydig/myoid cells, and the adult Leydig and myoid cells. Cells are colored according to their predicted locations along pseudotime. Neonatal data were from Sohni et al., 2019; 1-year-old and 25-year-old data were from Guo et al., 2018, and 7- to 14-year-old data were from Guo et al., 2020. See also FIG. 12.

FIG. 4G depicts the gene expression dynamics during specification of interstitial and Sertoli lineages. Deconvolution of the Monocle pseudotime plot according to ages/donors of origin. See also FIG. 12.

FIG. 5A depicts principal-component analysis of testicular niche progenitors from 6- and 7-week cells, revealing the existence of interstitial/Leydig and Sertoli lineage bifurcation.

FIG. 5B uses FIG. 5A to project and depict the expression of key transcription factors involving the specification of interstitial and Sertoli cells. Expression patterns of key factors that show specific patterns during the progenitor differentiation.

FIG. 5C depicts the key transcription factors involving the specification of interstitial and Sertoli cells. Staining of transcription factors GATA3 (cyan) in the 5- and 8-week samples.

FIG. 5D depicts the key transcription factors involving the specification of interstitial and Sertoli cells. Staining of transcription factors GATA4 (cyan) in the 6- and 17-week samples.

FIG. 5E depicts the key transcription factors involving the specification of interstitial and Sertoli cells. Co-staining of Sertoli (DMRT1, magenta) and germ cell (DDX4, cyan) markers in the 5- and 8-week samples.

FIG. 5F depicts the key transcription factors involving the specification of interstitial and Sertoli cells. Co-staining of 2 Sertoli cell markers, DMRT1 and SOX9, in the 5.5- to 17-week samples.

FIG. 6A depicts the proposed models for human germline development and somatic niche cell specification during prenatal and postnatal stages. Schematic summarizing the combined gene expression programs and cellular events accompanying human PGC differentiation into adult SSCs.

FIG. 6B depicts the proposed models for human germline development and somatic niche cell specification during prenatal and postnatal stages. The timeline and proposed model for human testicular somatic niche cell development at embryonic, fetal, and postnatal stages. Specification of a unique progenitor cell population toward Sertoli and interstitial/Leydig lineages begins at around 7 weeks postfertilization, when the cord formation occurs.

FIG. 7A depicts a single cell transcriptome profiling and analysis of the human fetal and postnatal testis. Partitioning the combined UMAP analysis in FIG. 1A based on the ages/donors of origin, with cells from each donor colored separately in different boxes. Related to FIG. 1A-1C.

FIG. 7B Top panel: depicts a single cell transcriptome profiling and analysis of the human fetal and postnatal testis. Bar graph showing the cell number of different cell types/clusters for each sample/age. Related to FIG. 1. Bottom panel depicts a single cell transcriptome profiling and analysis of the human fetal and postnatal testis. Bar graph showing the relative proportion of different cell types/clusters for each sample/age. Related to FIG. 1A-1C.

FIG. 8 depicts the expression patterns of additional markers (genes) projected on the UMAP plot Related to FIGS. 1A-1C.

FIG. 9A depicts the transition of human PGCs to State f0. Partitioning the combined tSNE analysis in FIG. 2A based on the ages/donors of origin, with cells from each donor colored separately in different panels. Related to FIG. 2A-2F.

FIG. 9B depicts the transition of human PGCs to State f0. Partitioning the combined tSNE analysis in FIG. 2A based on the ages/donors of origin, with cells from each donor colored separately in different panels. Related to FIG. 2A-2F

FIG. 9C depicts the transition of human PGCs to State f0. Partitioning the combined tSNE analysis in FIG. 2A based on the ages/donors of origin, with cells from each donor colored separately in different panels. Related to FIG. 2A-2F

FIG. 9D depicts the transition of human PGCs to State f0. Pseudotime trajectory (Monocle analysis) of embryonic, fetal, postnatal and adult germ cells. Cells are colored based according to the predicted pseudotime. Data from 7-day samples were from Sohni et al., 2019, and 1 year and adult data were from Guo et al., 2018. Related to FIGS. 2A-2F

FIG. 9E depicts the transition of human PGCs to State f0. Deconvolution of the Monocle pseudotime plot according to ages/donors of origin. Related to FIG. 2A-2F

FIG. 9F depicts the transition of human PGCs to State f0. H&E staining of section of a 5-week human embryo. Yellow arrow indicates genital ridge. Images were stitched per the protocol described in the Microscopy Methods section. Related to FIG. 2A-2F

FIG. 9G depicts the transition of human PGCs to State f0. Large field images of protein co-immunofluorescence for markers of proliferation (MKI67, yellow), pluripotency (NANOG, magenta) and germ cells (DDX4, cyan) in 5- and 8-week samples. Related to FIG. 2A-2F.

FIG. 9H depicts the transition of human PGCs to State f0. Large field images of protein co-immunofluorescence for germ cell (DDX4, magenta) and State 0 (PIWIL4, cyan) markers in samples from 12 to 16 weeks. Related to FIG. 2A-2F.

FIG. 10A depicts the network expression dynamic during fetal and postnatal germ cell development. Gene-gene network revealed by WGCNA analysis that are upregulated in PGC (3A), spermatogonia (3B) or State 0 (3C). The top ˜10 hub genes are highlighted. Related to FIG. 2A-2F.

FIG. 10B depicts the network expression dynamic during fetal and postnatal germ cell development. Gene-gene network revealed by WGCNA analysis that are upregulated in PGC (3A), spermatogonia (3B) or State 0 (3C). The top ˜10 hub genes are highlighted. Related to FIG. 2A-2F.

FIG. 10C depicts the network expression dynamic during fetal and postnatal germ cell development. Gene-gene network revealed by WGCNA analysis that are upregulated in PGC (3A), spermatogonia (3B) or State 0 (3C). The top ˜10 hub genes are highlighted. Related to FIG. 2A-2F.

FIG. 10D depicts the network expression dynamic during fetal and postnatal germ cell development. Expression patterns of the top hub genes project onto the tSNE plot from FIG. 2A. Related to FIG. 2A-2F.

FIG. 10E depicts the network expression dynamic during fetal and postnatal germ cell development. Expression patterns of the top hub genes project onto the tSNE plot from FIG. 2A. Related to FIG. 2A-2F.

FIG. 10F depicts the network expression dynamic during fetal and postnatal germ cell development. Expression patterns of the top hub genes project onto the tSNE plot from FIG. 2A. Related to FIG. 2A-2F.

FIG. 10G depicts the network expression dynamic during fetal and postnatal germ cell development. Violin plot showing the genes that were specifically expressed in State f0 cells. With a standard statistical cutoff (fold change>2 & p-value<0.05), 11 genes more highly expressed in State f0 compared to PGCs and State 0 were identified. After filtering out genes that also exhibit high expression in other SSC states (e.g. States 1-4), this yielded 2 genes that are State f0-specific, ID3 and GAGE12H. Related to FIG. 2A-2F.

FIG. 10H depicts the network expression dynamic during fetal and postnatal germ cell development. Composition of migrating, mitotic and mitotic-arrest fetal germ cells in samples from 4 to 25 weeks. The data is from Li et al., 2017. Related to FIG. 2A-2F.

FIG. 10I depicts the network expression dynamic during fetal and postnatal germ cell development. Violin plot to show the proportion/percentage expression levels of known PGC and germ cell markers in migrating, mitotic and mitotic-arrest fetal germ cells from Li et al., 2017. Related to FIG. 2A-2F.

FIG. 11A depicts the somatic niche cell specification at embryonic and fetal stages. Bar graph showing the cell number of different cell types/clusters in the testicular niche cells for each sample/age. Related to FIGS. 3A-3D, 4A-4G and 5A-5F.

FIG. 11B depicts the somatic niche cell specification at embryonic and fetal stages. Expression patterns of key factors (genes) that show specific patterns during progenitor differentiation. Related to FIGS. 3A-3D, 4A-4G and 5A-5F.

FIG. 11C depicts the somatic niche cell specification at embryonic and fetal stages. Large field for co-staining of two Sertoli cell markers, DMRT1 (cyan) and SOX9 (magenta), in the 8- to 17-week samples. Scale bars indicate 40 um. Related to FIGS. 3A-3D, 4A-4G and 5A-5F.

FIG. 11D depicts the somatic niche cell specification at embryonic and fetal stages. Co-staining pattern of the Leydig cell marker DMRT1 (cyan) and the Sertoli cell marker SOX9 (magenta) in the 8- to 18-week samples. Scale bars indicate 50 um. Related to FIGS. 3A-3D, 4A-4G and 5A-5F.

FIG. 11E depicts the somatic niche cell specification at embryonic and fetal stages. ACTA2 staining pattern in fetal (10W) and adult testis. Unlike in the adult testis where ACTA2+ myoid cells surround the seminiferous tubules, ACTA2 expression is limited in the fetal testis (boundaries of the cords marked by dashed line). Although it was possible to detect limited ACTA2 signal outside the fetal cords, the signal was sparse and the cells that express ACTA2 did not elongate and form a ring-like structure. Scale bars on the left: large field (50 μm), insert (10 μm). Scale bars on the right: 20 μm. Related to FIGS. 3A-3D, 4A-4G and 5A-5F.

FIG. 12A. Proposed models for human germline development and somatic niche cell specification during embryonic, fetal and postnatal stages. Representative genes that display differential expression patterns during Leydig cell differentiation from fetal to infant stages. Related to FIGS. 4A-4G and 5A-5F.

FIG. 12B. Proposed models for human germline development and somatic niche cell specification during embryonic, fetal and postnatal stages. Representative genes that display differential expression patterns during Sertoli cell differentiation from fetal to infant stages. Related to FIGS. 4A-4G and 5A-5F.

FIG. 12C. Proposed models for human germline development and somatic niche cell specification during embryonic, fetal and postnatal stages. Monocle analysis of 6- and 7-week somatic progenitors revealed developmental bifurcation. Related to FIGS. 4A-4G and 5A-5F.

FIG. 12D. Proposed models for human germline development and somatic niche cell specification during embryonic, fetal and postnatal stages. Pseudotime trajectory of the monocle plot in FIG. 12C. Related to FIGS. 4A-4G and 5A-5F.

FIG. 12E. Proposed models for human germline development and somatic niche cell specification during embryonic, fetal and postnatal stages. Expression patterns of key factors projected onto the Monocle plot in FIG. 12C. Related to FIGS. 4A-4G and 5A-5F.

FIG. 13 diagrammatically depicts the complex yet organized human spermatogonial stem cell niche.

FIG. 14. Protein co-immunofluorescence in cultured adult tissue for markers of germ cells (DDX4), DNA synthesis (EdU), and nucleic acid (DAPI) showing proliferation/replication of differentiating spermatogonia in vitro.

FIG. 15. Protein co-immunofluorescence in cultured seminal tubules for markers of differentiating spermatogonia (EdU+/UTF1−/SYCP3−), spermatocytes (EdU+/SYCP3+), and nucleic acid (DAPI). The clear presence of cells showing both replication (EdU+) and a strong marker of meiosis (SYCP3+) demonstrates the ability of differentiating spermatogonia to both undergo replication and enter meiosis.

FIG. 16. Immunostaining of cultured tissue and tissue obtained directly from a donor using haemotoxylin and Eosin staining.

FIG. 17. Left panel: dimension reduction presentation (via UMAP) of combined single-cell transcriptome data from fresh tissue, tissue cultured for 1 day, and tissue cultured for 4 days. Each dot represents a single cell and is colored according to its tissue of origin and is labeled with cell categories and colored according to its cell type identity. Right panels: expression patterns of selected markers projected on the UMAP plot. For each cell cluster, 1 cell marker (gene) is shown in the main figure.

FIG. 18. Left panel: dimension reduction presentation (via UMAP) of combined single-cell transcriptome data from fresh tissue, tissue cultured for 1 day, and tissue cultured for 4 days. Each dot represents a single cell and is colored according to its tissue of origin and is labeled with cell categories and colored according to its cell type identity. Right panel: Diagrammatic depiction of the spermatogonial stem cell niche for reference.

FIG. 19. Top left panel: the dimension reduction presentation (via UMAP) shown in FIG. 18 highlighting Leydig and myoid cells. Top right panel: k-means clustering of genes exhibiting differential expression (n=2,448). Each row represents a gene, and each column represents a single cell, with columns/cells arranged according to cell type. Differential gene expression levels use a Z score as defined by the color key; associated GO terms (using DAVID version 6.7) are given on the right of the corresponding gene clusters. Genes associated with the GO terms are shown in Table 3. Bottom panels: Violin-plots of expression levels for selected genes in Leydig, cultured, and myoid cells. Each dot represents the expression level within a single cell for the gene indicated on top of each panel.

FIG. 20. Top left panel: the dimension reduction presentation (via UMAP) shown in FIG. 18 highlighting endothelial cells. Top right panel: k-means clustering of genes exhibiting differential expression (n=2,448). Each row represents a gene, and each column represents a single cell, with columns/cells arranged according to cell type. Differential gene expression levels use a Z score as defined by the color key; associated GO terms (using DAVID version 6.7) are given on the right of the corresponding gene clusters. Genes associated with the GO terms are shown in Table 4. Bottom panels: Violin-plots of expression levels for selected key marker genes in cultured and endothelial cells. Each dot represents the expression level within a single cell for the gene indicated on top of each panel.

FIG. 21. Plot showing that somatic cells are more affected by culturing than germ cells

FIG. 22. Left panel: photograph of tissue cultured for 7 days in base conditions and base conditions supplemented with echinomycin. Right panel: Protein co-immunofluorescence in the cultured tissue for markers of spermatogonia (EdU) and nucleic acid (DAPI).

FIG. 23. EdU derivative fluorescence and protein co-immunofluorescence in tissue cultured for 7 and 14 days in the absence or presence of echinomycin for markers of germ cells (DDX4), spermatogonia (EdU), and nucleic acid (DAPI) showing proliferation/replication of differentiating spermatogonia in vitro, and the ability of differentiating spermatogonia to proliferate/replicate and enter meiosis.

FIG. 24. EdU derivative fluorescence and protein co-immunofluorescence in tissue cultured for 14 days in the absence or presence of echinomycin and echinomycin, testosterone, FSH, and RA for EdU, and nucleic acid (DAPI) showing proliferation/replication of differentiating spermatogonia in vitro, and the ability of differentiating spermatogonia to proliferate/replicate and enter meiosis.

FIG. 25. Flow chart depicting the process used to identify dysregulated biological pathways in cultured testicular tissue.

FIG. 26. Flow chart depicting the process of identifying dysregulated pathways in cultured testicular tissue.

FIG. 27. Flow chart illustrating a process for identification and selection of ligands capable of improving testicular germ cell proliferation (flow chart for selection of ligands of SSCs: Document #89989143).

FIG. 28. Dot plot showing enrichment results of receptors identified using CellPhoneDB analysis. The Python package CellPhoneDB was utilized with single-cell RNA-seq of adult testis atlas to uncover candidate signaling pathways activated in human Spermatogonial Stem Cells (SSCs). The top 14 non-redundant enriched ligand-receptor pairs for receptors expressed in SSCs are plotted. Data pertaining to four SSC receptors (FGFR3, BMPR2, RET, GFRA1) of known importance to SSC biology are also plotted to serve as positive controls. The darkness of each data point indicates the mean enrichment between each paired ligand and receptor, with darker coloring indicating higher enrichment and paler color indicating lower enrichment. The size of each data point indicates the relative ranking of each identified ligand-SSC receptor pair, with larger size indicating higher ranking.

FIG. 29. EdU derivative fluorescence in seminal tubules cultured for 21 days for markers of DNA synthesis (EdU) and nucleic acid (DAPI) showing proliferation/replication of germ cells. Left panel: Cells cultured in base media. Right panel: Cells cultured in the improved culture media of Table 6, with improvements prompted by the analysis of the single cell data of cultured tubules.

FIG. 30. Dot plot showing enrichment results of dysregulated pathways identified using the iterative process of the instant disclosure. The darkness of each data point indicates the statistical significance of the enrichment of that term within a given gene set, with darker coloring indicating higher significance and paler color indicating lower significance. The size of each data point represents the gene count, which is the number of genes associated with a specific GO term. A larger point indicates a higher gene count.

FIG. 31. EdU derivative fluorescence in cultured seminal tubules for markers of DNA synthesis (EdU) showing the level of proliferating germ cells in tissue cultured in a first culture condition (top panels) and the level of proliferating germ cells in tissue cultured in the first culture condition further comprising an inhibitor discovered to be effective at improving germ cell proliferation (bottom panels). Bright dots: proliferating germ cells.

FIG. 32. EdU derivative fluorescence and protein immunofluorescence in cultured spermatogonia (in C2 medium) for markers of germ cells (DDX4), DNA synthesis (EdU), and nucleic acid (DAPI) showing the level of proliferating germ cells in spermatogonia cultured for seven days (top panel) and 14 days (bottom panel). Arrows point to proliferating spermatogonia.

FIG. 33. Protein immunofluorescence in cultured spermatogonia for markers of germ cells (DDX4) and nucleic acid (DAPI) showing the level of proliferating germ cells in spermatogonia cultured for 14 days in C2 media (top panels) and C2 media with 8× testosterone (bottom panels). Arrows indicate clusters of germ cells. Boxes indicated areas with magnified images.

FIG. 34. Bar graph showing duration of proliferation of SPG cells cultured using the hybrid culture system. The graph shows cell count of SPG at days 2, 7, 14, and 21 days after a 7-day culture period in tubules. Culture conditions are listed below the X axis. An explanation of abbreviations is also shown.

FIG. 35. EdU derivative fluorescence and protein immunofluorescence in cultured SPG (in C2 medium) for markers of germ cells (DDX4) and DNA synthesis (EdU) showing the level of proliferating germ cells in spermatogonia cultured for 14 days (top panel) and 21 days (bottom panel). SPG were prepared from 7-day cultured tissue using digestion with Col IV and Dispase followed by culture in C2 media supplemented with GSH, VA, and VE. Arrows point to proliferating spermatogonia. Culture conditions are 2AO-dg2, described in FIG. 34.

FIG. 36 diagrammatically depicts the stages during differentiation of adult spermatogonial stem cells (SSCs) into mature spermatozoa.

### DETAILED DESCRIPTION

The present disclosure encompasses processes for identifying culture conditions that support growth and development of testicular germ cells in vitro, both germline and somatic. The processes make use of genomic approaches to identify testicular germ cell proliferation factors by identifying dysregulated biological pathways in cultured cells and receptor ligands that can be used to identify the in vitro culture conditions. Surprisingly, it was discovered that culture conditions identified using the process of the instant disclosure faithfully recreate conditions needed for stages of spermatogenesis and maintaining the identity, growth, and survival of the testicular germ cells in vitro. Accordingly, media and cell cultures comprising the identified culture conditions that can be used to support testicular germ cell growth in vitro are also disclosed. The processes and compositions can comprise spermatogonial stem cells and spermatogonia grown in vitro that can be used for infertility treatment.

### I. Process for Identifying Culture Conditions

One aspect of the present disclosure encompasses processes for identifying culture conditions supportive of testicular germ cell proliferation in vitro. The processes comprise identifying factors (proliferation factors) that, when added to culture media, can improve the level of proliferation of testicular germ cells cultured in the medium. Optionally, the processes can be iteratively repeated to identify additional proliferation factors, or to identify combinations and concentrations of factors optimized for germ cell proliferation.

The process can identify culture conditions for germ cell growth and development all while maintaining the identity and survival of the germ cells. Culture conditions identified using processes of the instant disclosure can be utilized to obtain sperm with low rates of deleterious or de novo mutations or epigenetic perturbations. Further, the process can be used to help treat male infertility by manipulating germline, help restore fertility for childhood cancer survivors, and provide a useful platform to study human germline.

### (a) Testicular Tissue and Germ Cells

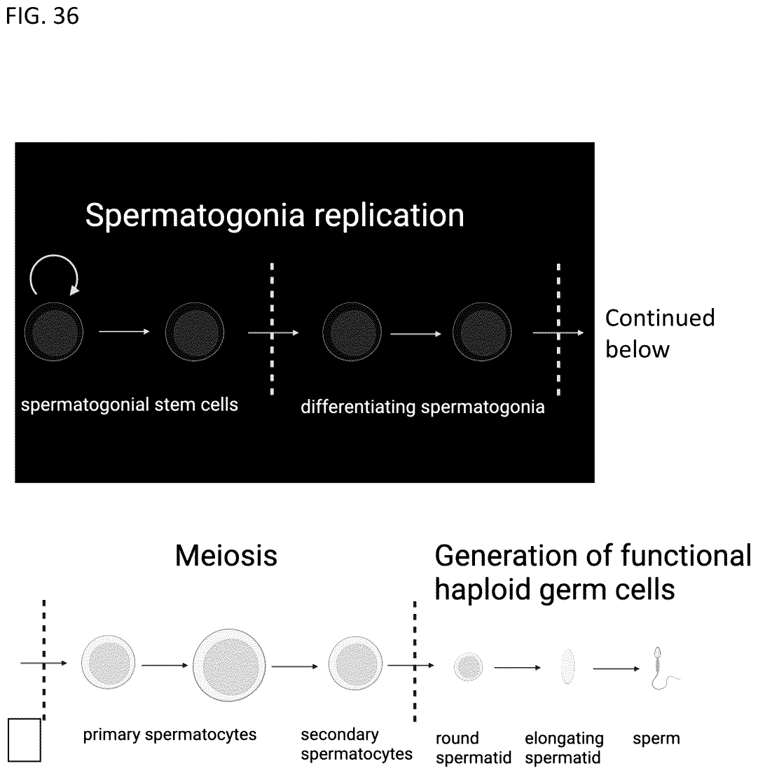

Mammalian spermatogenesis involves the differentiation of adult spermatogonial stem cells (SSCs) into mature spermatozoa through a complex developmental process (FIG. 36), regulated by the testis niche in the seminiferous tubules. SSCs denote undifferentiated male germ cells that have the potential to self-renew and differentiate into committed progenitors that maintain spermatogenesis throughout adult life. SSCs must carefully balance their self-renewal and differentiation, and then undergo niche-guided transitions between multiple cell states and cellular processes-including a commitment to mitosis, meiosis, and the subsequent stages of sperm maturation, which are accompanied by chromatin repackaging and major morphological changes. Spermatogenesis further comprises the generation of spermatocytes and spermatids, and maturation of spermatids to spermatozoa.

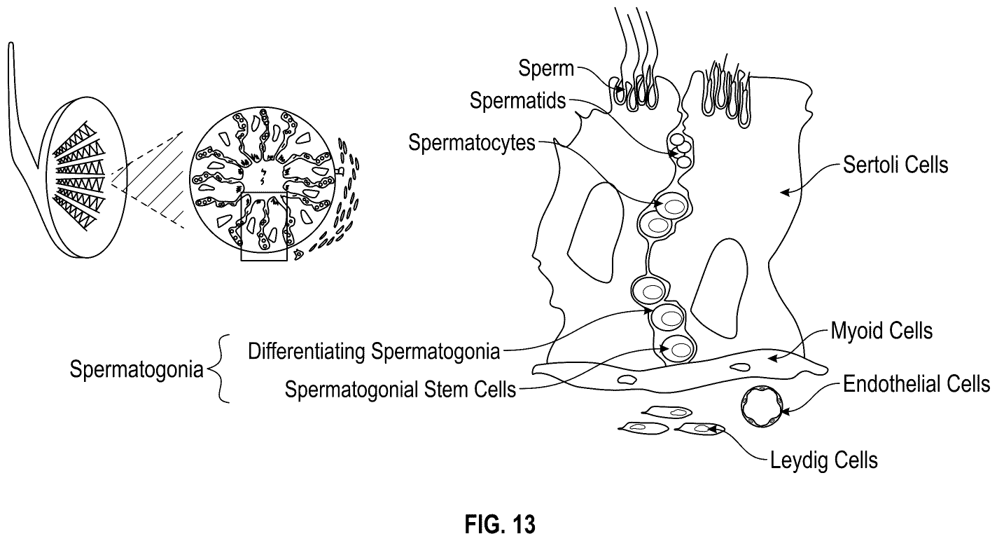

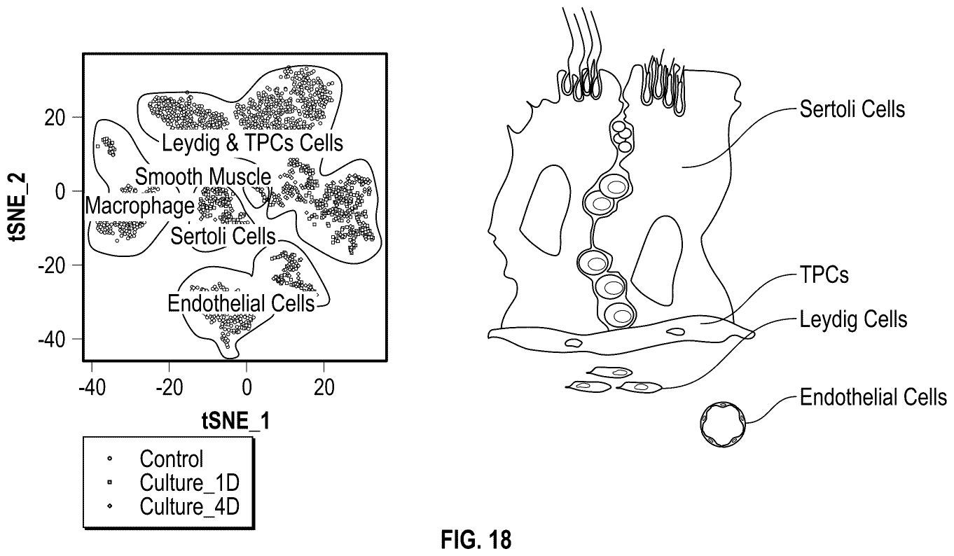

The overall efficiency and success of spermatogenesis relies on the presence of the stem cell niche. FIGS. 13 and 18 diagrammatically depicts the spermatogonial stem cell niche, showing various cell types, including Sertoli cells, Leydig cells, endothelial cells, and myoid cells also sometimes referred to as peritubular myoid cells. This relationship between the developing germ cells and the surrounding testicular environment allows for the correct spatial arrangement of cells and enables them to receive and interpret the various signals and factors necessary for spermatogonial stem cell self-renewal and germ cell differentiation. “Sertoli cells,” as used herein, refer to cells of the mammalian testis that are responsible for providing immune privilege. Sertoli cells are considered to be “nurse” or “chaperone” cells because they immunoprotect and assist in the development of germ cells into spermatozoa. The Sertoli cell maintains the “blood-testis” barrier (BTB) by forming occluding junctions that separate the tubules that comprise the seminiferous epithelium into two compartments. “Leydig cells,” as used herein, refer to the cells in the mammalian testis that contain two key steroidogenic enzyme pathways, namely, cytochrome P450 side chain cleavage (P450scc) and 3β-HSD. Leydig cells carry out the conversion of cholesterol, the substrate for all steroid hormones, to pregnenolone; and the conversion of pregnenolone to progesterone. “Peritubular cells” or “peritubular myoid cells” refer to myofibroblast-like cells that surround the seminiferous tubules and are responsible for tubular contractility and sperm transport.

A process of the instant disclosure comprises identifying culture conditions supportive of testicular germ cell proliferation in vitro. As used herein, the term “testicular germ cell” refers to testicular germ cells at any stage of development from SSCs to mature spermatozoa. Accordingly, a process of the instant disclosure can identify culture conditions supportive of SSC self-renewal and differentiation as well as transitions between multiple cell states and cellular processes during spermatogenesis.

The stages of development can be identified and verified by the distinct transcriptional/developmental states of germ cells, or by identification of markers specific for each cell type. The identity of germ cells at each stage of development can be identified using methods known in the art and can be as described in Guo et al., Cell Stem Cell, 2017; Guo et al., Cell Research, 2018; and Guo et al., Cell Stem Cell, 2020, the disclosures of all of which are incorporated herein in their entirety.

The process comprises culturing testicular tissue comprising germ cells in vitro in a culture medium. Testicular tissue can be tissue comprising germ cells isolated from a subject, or can be testicular tissue produced in vitro, such as testicular organoids comprising germ cells. If the testicular tissue is an organoid, the organoid can assume the function of the testis niche in guiding testicular germ cells during spermatogenesis. The process can also comprise culturing testicular germ cells in vitro in a culture medium. Accordingly, testicular germ cells can comprise isolated germ cells, germ cells associated with testicular tissue, or germ cells associated with organoids. In some aspects, the testicular tissue is tissue dissected from a subject and comprising seminiferous tubules and testicular germ cells. In some aspects, the testicular tissue is seminiferous tubules comprising germ cells. In some aspects, the process comprises culturing isolated SSCs and/or spermatogonia.

A process of the instant disclosure can be used to identify culture conditions that can support division, growth, and development (proliferation) of testicular germ cells in vitro at any stage in the developmental process. As used herein, the term “proliferation” when referring to testicular germ cells refers to the ability of germ cells to grow, divide, survive (e.g., dish life of stem cells), and develop, as well as maintain the identity and survival of the germ cells. Accordingly, the process can identify culture conditions that support self-renewal and transition of SSCs to proliferative spermatogonia, from proliferative spermatogonia to entering meiosis, from differentiating spermatogonia to primary and secondary spermatocytes, spermatids, through sperm maturation, or any combination thereof. In some aspects, the process is used to identify culture conditions supportive of SSC and spermatogonial proliferation.

The process can be used to identify culture conditions that can support proliferation of testicular germ cells of any mammalian animal in vitro. “Mammalian,” as used herein refers to both human subjects (and cells sources) and non-human subjects (and cell sources or types), such as dog, cat, mouse, monkey, etc. (e.g., for veterinary purposes). In some aspects, a process of the instant disclosure is used to identify culture conditions that can support proliferation of human testicular germ cells in vitro. This is important because there are no current successful ways of successfully culturing human testicular germ cells.

The testicular tissue or germ cells can be from prepubertal or adult fertile or infertile males. The testicular tissue or germ cells can be from a live subject or a cadaveric subject. The testicular tissue or germ cells can also be freshly harvested or can be cryopreserved cells. For instance, the cryopreserved testicular tissue or germ cells can be from a subject expected to have germ-damaging treatment (e.g., chemotherapy) for future use with assisted reproductive technologies. In some aspects, testicular germ cells can be from a pre-pubertal subject.

### (b) Identifying Factors of Dysregulated Pathways

One aspect of a process of the instant disclosure encompasses identifying one or more factors supportive of testicular germ cell proliferation in vitro by identifying one or more dysregulated pathways in cells of cultured testicular tissue, identifying factors that can regulate the dysregulated pathways (referred to hereinafter as pathway factors), and identifying among the pathway factors, factors that improve the level of proliferation of testicular germ cells by screening the identified pathway factors for factors that can regulate the identified dysregulated pathways to identify the factors among them that can improve germ cell proliferation. The inventors discovered that testicular cells cultured in media that cannot support sufficient germ cell proliferation comprise dysregulated biological pathways. The inventors also discovered that some factors that can regulated identified dysregulated pathways can improve proliferation of germ cells when the factors are used to supplement culture media.

A process of identifying dysregulated pathways comprises the use of a genomic approach to identify dysregulated pathways in the cultured testicular tissue or testicular germ cells. A process of the instant disclosure further comprises screening factors that can regulate the identified dysregulated pathways to identify the factors among them that can improve germ cell proliferation. Factors that can regulate the biological pathways and methods of identifying factors that can regulate pathways are known in the art or can be identified by individuals of skill in the art using known methods. The factors can be ligands, inhibitors, small molecule factors, as well as peptides among others. Screening the identified factors can be as described in Section I(d) herein below. In some aspects, identified pathway factors for screening are as listed in Table 7 herein below.

In some aspects, identifying the one or more dysregulated pathways in testicular cells cultured in a first set of culture conditions comprises (1) culturing testicular germ cells or testicular tissue comprising germ cells in vitro in a first culture medium, wherein the first culture medium supports a first level of proliferation of the germ cells; (2) profiling transcriptomes of testicular cells obtained from the testicular tissue; and (3) identifying RNA transcripts differentially expressed in cells grown in the first culture medium when compared to RNA transcripts expressed in cells obtained from control tissue. The differentially expressed RNA transcripts identify one or more dysregulated biological pathways in the testicular cells cultured in the first set of culture conditions when compared to the biological pathways of the control tissue or cells. Testicular tissues and cells can be as described in Section I(a) herein above.

In some aspects, the process further comprises assigning a cell type to each single testicular cell using cell type-specific gene markers expressed in each cell. When the process comprises assigning a cell type to single testicular cells, the process can further comprise identifying RNA transcripts differentially expressed in each cell type when compared to RNA transcripts expressed in cells of corresponding cell types obtained from control tissue.

As explained above, testicular germ cells can comprise isolated germ cells independent of other testicular tissue, germ cells associated with testicular tissue cultured in vitro, or germ cells associated with organoids in vitro. Accordingly, if the process comprises identifying factors by identifying dysregulated pathways in cells of cultured testicular tissue or in cells of cultured organoids, the process can comprise identifying one or more dysregulated pathways in cells of cultured testicular tissue or cells of cultured organoids and identifying dysregulated pathways of germ cells associated with testicular tissue or organoids. In some aspects, a process of the instant disclosure encompasses identifying dysregulated pathways in cells that form a niche for germ cell proliferation. For instance, the process can comprise identifying dysregulated pathways in somatic cells, including Sertoli cells, Leydig cells, endothelial cells, myoid cells, or any combination thereof. In some aspects, a process of the instant disclosure encompasses identifying dysregulated pathways in cells of seminiferous tubules, and germ cells in the seminiferous tubules. In some aspects, the process comprises identifying one or more factors that improve the level of proliferation of testicular germ cells cultured in vitro independently from other testicular tissue by identifying one or more dysregulated pathways in the cultured germ cells.

The dysregulated pathways are identified by first culturing testicular tissue or germ cells in vitro in a first culture medium that can support a first level of proliferation of testicular germ cells. In some aspects, the first culture medium is a base medium. The base medium can be as described in Section II herein below. In some aspects, the first culture medium comprises base medium supplemented with factors identified in a previous round of pathway factor-identification using the process of the instant disclosure and various combinations and concentrations of the previously identified pathway factors (See, e.g., Section I(d) herein below).

After culturing the testicular tissue or germ cells in the first culture medium, a genomics approach is used to identify the dysregulated pathways. In essence, identifying dysregulated pathways comprises identifying differentially expressed RNA transcripts in single testicular cells grown in vitro in the first culture medium when compared to RNA expressed in control tissue or germ cells. In some aspects, the genomes of cultured testicular tissue or cells are profiled to obtain the transcriptional profile of each isolated cell. When the process further comprises assigning a cell type to each single testicular cell, the transcriptional profile can be the transcriptional profile of each cell type. In some aspects, control testicular tissue is testicular tissue obtained directly from a subject. In some aspects, control testicular tissue is tissue directly obtained from a subject and the level of RNA transcripts in each cell type in the tissue obtained directly from a subject can be as described in Guo et al. 2018, the disclosure of all of which is incorporated herein in its entirety.

Any method of transcriptional profiling can be used in a process of the instant disclosure. Methods of obtaining transcriptional profiles of single cells are known in the art and include, without limitation, single-cell RNA sequencing (scRNA-seq), spatial transcriptomics, Single-Cell Combinatorial Indexing RNA Sequencing (sci-RNA-seq), and Single-nucleus RNA sequencing (snRNA-seq). scRNA-seq involves the isolation of individual cells, followed by reverse transcription of RNA to create a cDNA library which is then sequenced. In spatial transcriptomics, RNA transcripts are sequenced without first isolating single cells, allowing for the preservation of the spatial context of the transcriptome in tissue sections. The cells' transcriptomes can be related to their spatial position within the tissue. Single-Cell Combinatorial Indexing RNA Sequencing (sci-RNA-seq) allows for high-throughput single-cell transcriptome profiling. It works by using combinatorial labeling of cells and pooling steps to greatly increase the throughput. Single-nucleus RNA sequencing (snRNA-seq) is used when the cells are difficult to dissociate or are sensitive to the process (e.g. Sertoli cells can be too large), or when the analysis of archived frozen material is required. It profiles the transcriptome of single nuclei rather than whole cells. In some aspects, the cells are dissociated to obtain single cells of all types of testicular tissue cell types of the tissue and the genomes of isolated cells are profiled to obtain the transcriptional profile of each isolated cell.

In some aspects, the profiling step of the process comprises using scRNA-seq approaches to identify the differentially expressed RNA transcripts in each cell type when compared to the level of RNA transcripts in the corresponding cell type in control tissue or germ cells, such as, from tissue obtained directly from a donor. For instance, identification of differentially expressed RNA transcripts using scRNA-seq can be as described in Guo et al., Cell Stem Cell, 2017; Guo et al., Cell Research, 2018; and Guo et al., Cell Stem Cell, 2020, the disclosures of all of which are incorporated herein in their entirety.

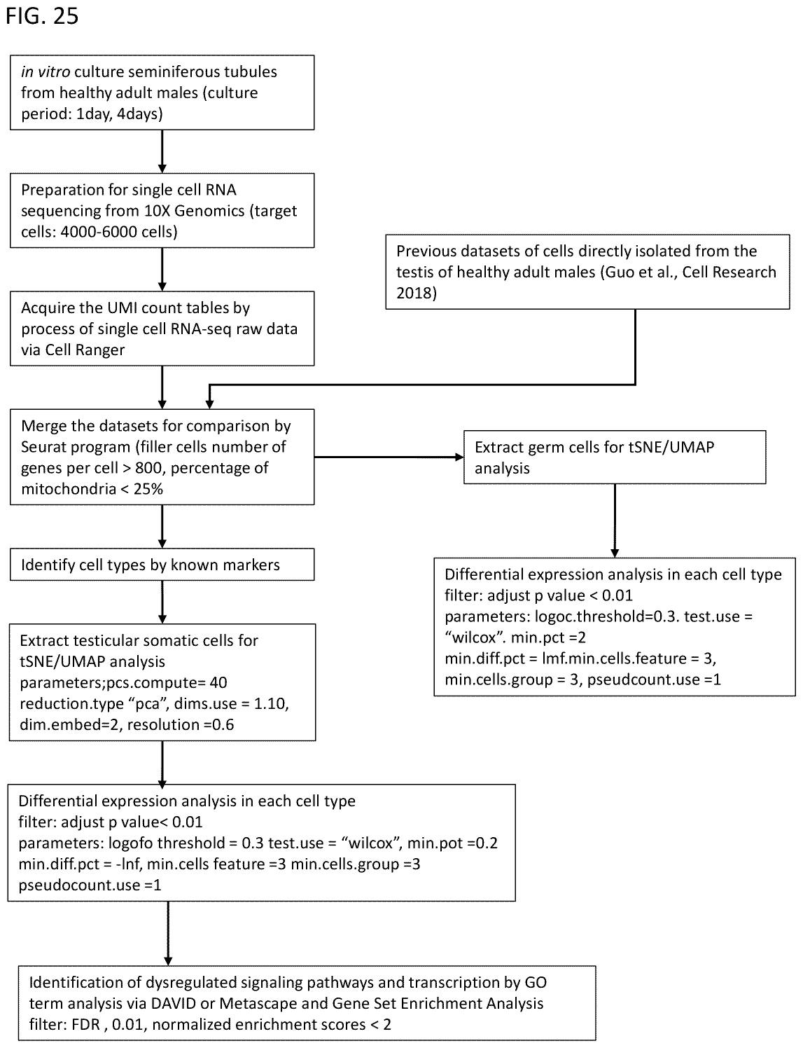

The differentially expressed RNA transcripts identify one or more dysregulated biological pathways in the in vitro cultured cells. In some aspects, Gene Ontology (GO) terms of genes expressing the RNAs in the cells or cell types is used to group differentially expressed genes into pathways. In some aspects, dysregulated pathways in cultured testicular tissue can be identified using a process detailed in the diagram shown in FIG. 25. In some aspects, the biological pathways are identified using methods described in Example 2 herein below.

A dysregulated biological pathway can be any biological pathway essential for testicular germ cell proliferation. For instance, the identified dysregulated pathways can be metabolic pathways, transcription pathways, signaling pathways, survival pathways, cell cycle pathways, physiological pathways, and developmental pathways among others. In some aspects, dysregulated pathways are identified in cultured testicular tissue, and the identified dysregulated pathways are pathways associated with extracellular exosome, negative regulation of apoptotic process, cytokine, response to hypoxia, actin cytoskeleton, extracellular matrix, and muscle contraction.

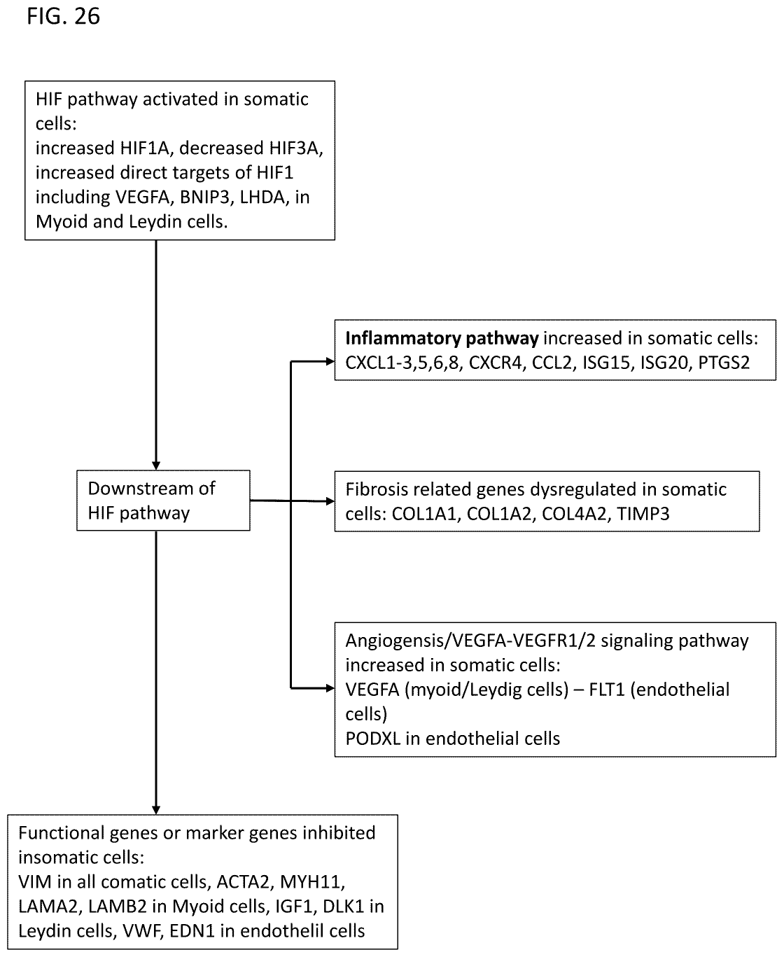

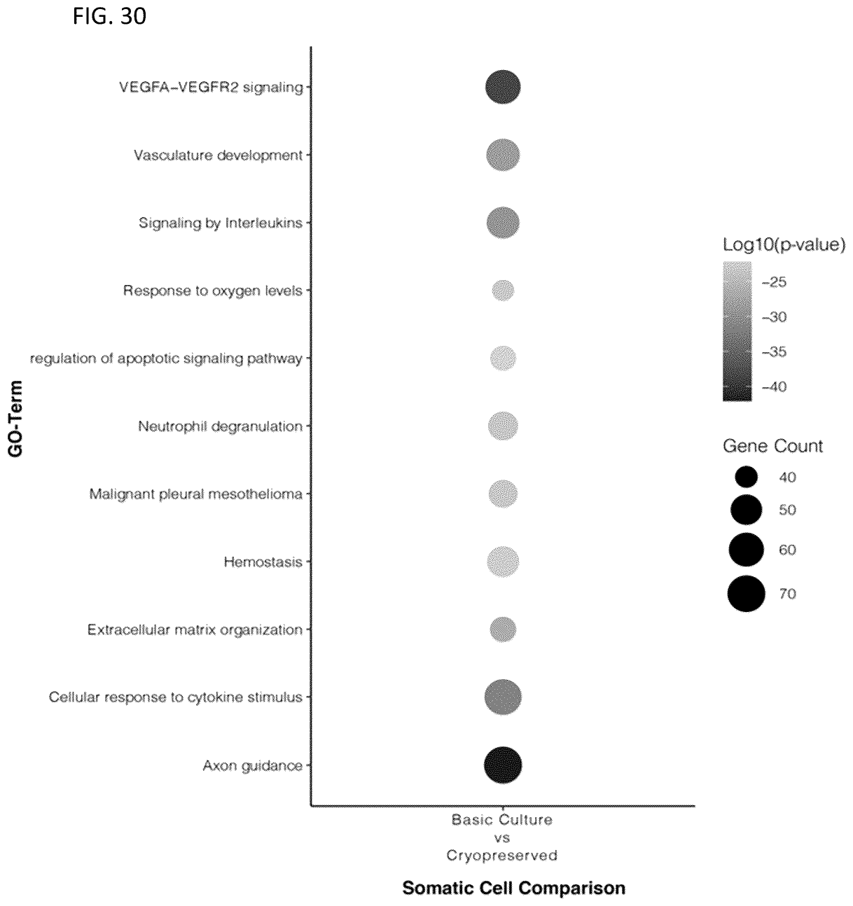

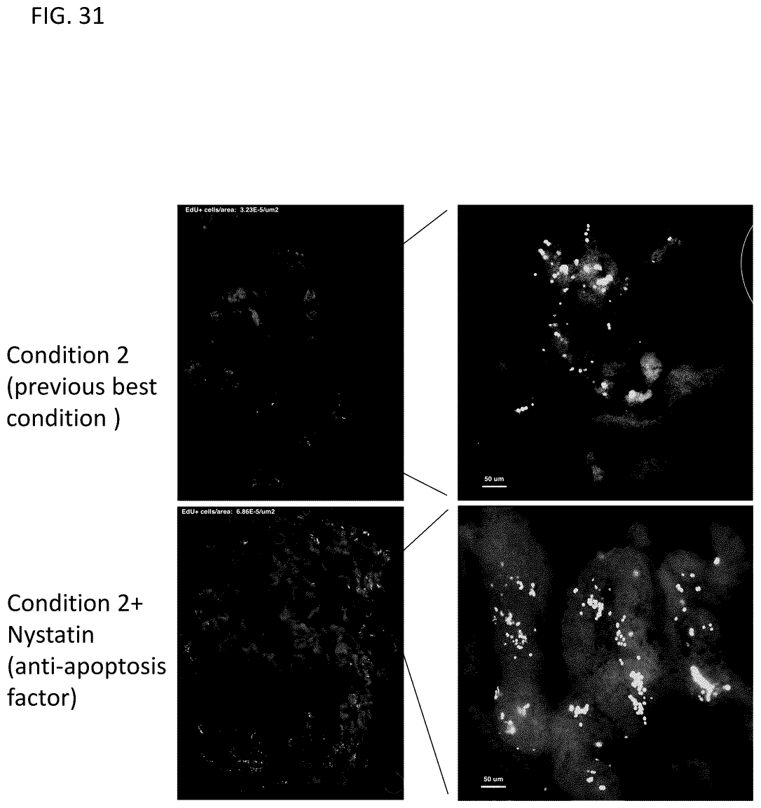

In some aspects, the one or more dysregulated pathways comprise one or more pathways of hypoxia-inducible factor (HIF; FIG. 26). In some aspects, the one or more dysregulated pathways comprise one or more pathways of apoptosis (FIG. 30 and FIG. 31). In some aspects, dysregulated pathways are identified in cultured testicular tissue, and the identified dysregulated pathways are as listed in Tables 3 and 4. In some aspects, dysregulated pathways are identified in cultured testicular tissue, and the identified dysregulated pathways are pathways associated with response to hypoxia.

### (c) Identify Receptor Ligands

Another aspect of the instant disclosure encompasses identifying one or more factors supportive of testicular germ cell proliferation in vitro by identifying ligands of receptors that can improve germ cell proliferation in testicular tissue or germ cells cultured in vitro. In essence, the process comprises identifying one or more receptors expressed in testicular tissue or germ cells, identifying ligands of the identified receptors, and screening the identified ligands in testicular tissue or germ cells cultured in vitro to identify receptor ligands that can improve germ cell proliferation in in vitro cultures of testicular tissue or germ cells.

Cell surface receptors play crucial roles in the proliferation and development of testicular germ cells, the precursors to sperm cells. Receptors mediate the signaling pathways that regulate germ cell growth, differentiation, and function. The binding of specific ligands, such as hormones or growth factors, to these cell surface receptors triggers intracellular signaling cascades, influencing gene expression and subsequent cellular behavior. For instance, follicle-stimulating hormone (FSH) receptors and luteinizing hormone (LH) receptors, both present on the cell surface, are critically involved in the endocrine regulation of spermatogenesis. FSH stimulates Sertoli cells, which support and nourish developing germ cells, while LH acts on Leydig cells to stimulate the production of testosterone, a hormone essential for spermatogenesis. Additionally, growth factors like glial cell line-derived neurotrophic factor (GDNF), acting through its receptor GFRα1, play vital roles in the self-renewal of spermatogonial stem cells.

A process of identifying receptors expressed in testicular tissue and germ cells comprises the use of a genomic approach. Testicular tissues and cells can be as described in Section I(a) herein above. In some aspects, databases of receptor expression patterns can be mined to identify receptors expressed in testicular tissue or germ cells. For instance, receptors expressed in testicular tissue and cells can be identified by identified receptor genes comprising a level of expression indicative of substantial expression in the cells and/or an expression pattern indicative of testicular tissue-specific or testicular cell-type specific expression. Any database of receptor expression and ligands can be used in a process of the instant disclosure. Databases of receptors and ligands are known in the art. Further, it will be recognized that databases continue to be updated and new databases continue to be compiled. Accordingly, a database of the instant disclosure can be an existing database of receptors and ligands as well as databases compiled or updated in the future. Non-limiting examples of databases of receptors and ligands suitable for use in a process of the instant disclosure include CellphoneDB, CellChat, huARdb (human Antigen Receptor database; Wu et al., Nucleic Acids Research, Volume 50, Issue D1, 7 Jan. 2022, Pages D1244-D1254), Cellinker (Zhang et al., Bioinformatics. 2021 Jan. 20), among others. It will be noted that, as these databases may not necessarily encompass all expressed receptors at all stages of development of all tissues and cells, a process of the instant disclosure, could identify different receptors and ligands depending on the database used in the process and depending on the testicular tissue or germ cells and developmental stages of testicular tissue and germ cells being investigated. As shown in Example 3 herein below, extensive experimentation showed that other methods of identifying receptors and corresponding ligands such as using CellphoneDB and CellChat to analyze cell-cell interaction to find potential ligands generated different results depending on the receptor ligand database used.

In some aspects, the process can comprise identifying receptors in cells of testicular tissue or cells of cultured organoids. In some aspects, a process of the instant disclosure encompasses identifying receptors in cells that form a niche for germ cell proliferation. For instance, the process can comprise identifying receptors expressed in somatic cells, including Sertoli cells, Leydig cells, endothelial cells, myoid cells, or any combination thereof. In some aspects, a process of the instant disclosure encompasses identifying receptors in cells of seminiferous tubules. In some aspects, the process comprises identifying receptors of testicular germ cells. In some aspects, the receptors are identified in SSCs, differentiating spermatogonia, or both. In some aspects, the receptors are identified in State 1 SSCs (active stem cells) and State 2/3 differentiating spermatogonia (differentiated germ cells with strong proliferation ability).

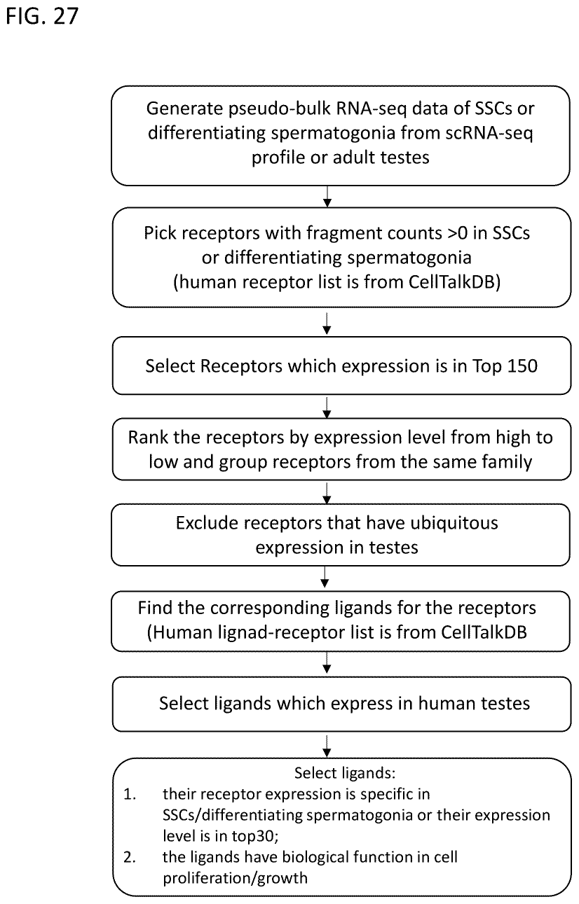

In some aspects, the receptors are identified by determining the level of expression of RNA transcripts of receptor genes expressed in the various cells and cell types of interest. In some aspects, the level of expression of RNA transcripts in testicular cells can be as described in Guo et al. 2018, the disclosure of all of which is incorporated herein in its entirety. In some aspects, identifying ligands can be as depicted in FIG. 27. In some aspects, receptors and corresponding ligands are identified as described in Example 3. In some aspects, the identified receptors can be as listed in Table 5.

### (d) Identifying Proliferation Factors

A process of the instant disclosure further comprises identifying among the pathway factors that can regulate the dysregulated pathways in Section I(b) or the receptor ligands identified in Section I(c) the one or more factors that can improve the level of proliferation of testicular germ cells in vitro. As used herein, the term “improved proliferation” when referring to testicular germ cells refers to improved growth, division, survival, physiology, or development of the germ cells and can be measured by an increase in the number of germ cells over time during culture, the duration of life of the cells in the culture medium, proper physiology of the cells, or any combination thereof.

Identifying one or more proliferation factors comprises culturing testicular tissue in vitro in culture media supplemented with one or more factors that regulate that can regulate the dysregulated pathways in Section I(b), supplemented with the receptor ligands identified in Section I(c), or any combination of the factors and ligands. The second cultures that support improved levels of germ cell proliferation comprise one or more proliferation factors, thereby identifying the one or more factors that improve the level of proliferation of testicular germ cells. In some aspects, improved proliferation of testicular germ cells in the new culture conditions can be confirmed by testing for improved growth, division, survival, physiology, or development of the germ cells and can be measured by an increase in the number of germ cells over time during culture, the duration of life of the cells in the culture medium, proper physiology of the cells, or any combination thereof. Germ cells grown in the identified culture conditions can maintain their identity at all stages of development. When the testicular cells comprise cells other than germ cells such as cells other than germ cells in tissue obtained from subjects or cells other than germ cells in organoids, cells grown in the identified culture conditions can maintain their identity at all stages of development. In some aspects, improved proliferation of testicular germ cells in the new culture conditions comprise maintained testicular size and germ cell division and survival. Culture media can be as described in Section II herein below and culturing testicular tissue comprising testicular germ cells or isolated testicular germ cells can be as described in Section III herein below.

In some aspects, identifying culture conditions can be informed by a previously performed round of the process. More specifically, identifying culture conditions comprises can be iteratively repeating to identify additional proliferation factors, various combinations of identified factors, various levels of the factors, and any combination thereof. When replication factors are identified by identifying dysregulated pathways, the process of identification of dysregulated pathways and pathway factors, screening the identified pathway factors, or any combination thereof can be iteratively repeated to identify proliferation factors to identify additional factors, various combinations of identified factors, various levels of the factors, and any combination thereof that improve the level of proliferation of testicular germ cells can be iteratively repeated to identify additional factors that improve the level of proliferation of testicular germ cells.

Importantly, the inventors discovered that a process of identifying proliferation factors using testicular tissue can be used as a learning platform for identifying proliferation factors that can improve proliferation of germ cells grown in vitro in an alternative growth format. More specifically, proliferation factors identified using a process that uses testicular tissue to identify proliferation factors can in turn be used in a process of identifying proliferation factors using isolated testicular germ cells, thereby greatly increasing the efficiency of identifying the germ cell proliferation factors.

In some aspects, proliferation factors identified using a process of the instant disclosure can be as described in Section II herein below. In some aspects, ligands of the identified receptors can be as described in Section II herein below.

### (e) Aspects of Processes

One aspect of the instant disclosure encompasses an iterative process for identifying culture conditions supportive of testicular germ cell proliferation in vitro. The process first comprises identifying one or more dysregulated pathways in testicular cells cultured in a first set of culture conditions. In some aspects, identifying one or more dysregulated pathways comprises culturing testicular tissue in vitro in a first culture medium, profiling transcriptomes of single testicular cells obtained from the testicular tissue, and identifying RNA transcripts differentially expressed in each cell type when compared to RNA transcripts expressed in cells of corresponding cell types obtained from control tissue. Culturing testicular tissue comprising testicular germ cells or isolated testicular germ cells can be as described in Section III herein below.

After identifying pathways dysregulated in testicular cells cultured in the first set of culture conditions, the process then comprises identifying one or more proliferation factors that improve the level of proliferation of testicular germ cells. Identifying one or more proliferation factors that improve the level of proliferation comprises culturing testicular tissue in vitro in one or more second culture media, wherein the one or more second culture media comprise the first culture medium supplemented with one or more factors that regulate an identified biological pathway. The tissue and cells are then cultured and one or more second culture media that support improved levels of germ cell proliferation when compared to the first level of germ cell proliferation in the first culture medium are identified. The second cultures that support improved levels of germ cell proliferation comprise one or more proliferation factors, thereby identifying the one or more factors that improve the level of proliferation of testicular germ cells.

In some aspects, the process of identification of dysregulated pathways and pathway factors, screening the identified pathway factors, or any combination thereof can be iteratively repeated to identify proliferation factors to identify additional factors, various combinations of identified factors, various levels of the factors, and any combination thereof that improve the level of proliferation of testicular germ cells can be iteratively repeated to identify additional factors that improve the level of proliferation of testicular germ cells.

In some aspects, the process of identification of receptors and receptor ligands, screening the ligands, or any combination thereof can be iteratively repeated to identify proliferation factors to identify additional factors, various combinations of identified factors, various levels of the factors, and any combination thereof that improve the level of proliferation of testicular germ cells.

In some aspects, the dysregulated pathway is selected from the HIF pathway, pathways downstream of the HIF pathway, and any combination thereof. In some aspects, the dysregulated pathway comprises the HIF pathway, inflammatory pathways, fibrosis pathways, angiogenesis/VEGFA-VEGFR1/2 signaling pathways, and any combination thereof. In some aspects, the one or more dysregulated pathways comprise one or more pathways of hypoxia-inducible factor (HIF; FIG. 26). In some aspects, dysregulated pathways are identified in cultured testicular tissue, and the identified dysregulated pathways are as listed in Tables 3 and 4. In some aspects, dysregulated pathways are identified in cultured testicular tissue, and the identified dysregulated pathways are pathways associated with response to hypoxia. In some aspects, proliferation factors identified using a process of the instant disclosure can be as described in Section II herein below.

Another aspect of the instant disclosure encompasses an iterative process for identifying culture conditions supportive of testicular germ cell proliferation in vitro. The process comprises identifying one or more dysregulated pathways in testicular cells cultured in a first set of culture conditions by: culturing testicular tissue in vitro in a first culture medium, wherein the testicular tissue comprises seminiferous tubules and testicular germ cells, and wherein the first culture medium supports a first level of proliferation of germ cells; profiling transcriptomes of single testicular cells obtained from the testicular tissue using single cell RNA sequencing (scRNA-seq); assigning a cell type to each single testicular cell using cell type-specific gene markers expressed in each cell; and identifying RNA transcripts differentially expressed in each cell type when compared to RNA transcripts expressed in cells of corresponding cell types obtained from control tissue, wherein differentially expressed RNA transcripts identify one or more dysregulated biological pathways in the testicular cell types cultured in the first set of culture conditions.

The process also comprises identifying one or more proliferation factors that improve the level of proliferation of testicular germ cells in vitro by: culturing testicular tissue in vitro in one or more second culture media, wherein the one or more second culture media comprise the first culture medium supplemented with one or more identified factors that regulate one or more of the identified biological pathways; identifying one or more second culture media that support improved levels of germ cell proliferation when compared to the first level of germ cell proliferation in the first culture medium, thereby identifying the one or more factors that improve the level of proliferation of testicular germ cells. The culture conditions that support testicular germ cell proliferation comprise culture media supplemented with one or more of the identified factors. Culture media can be as described in Section II herein below and culturing testicular tissue comprising testicular germ cells or isolated testicular germ cells can be as described in Section II herein below.

The process also comprises iteratively repeating the steps of pathway identification and screening factors that can regulate the identified pathways to identify additional factors that improve the level of proliferation of testicular germ cells.

An additional aspect of the instant disclosure encompasses an iterative process for identifying culture conditions supportive of testicular germ cell proliferation in vitro. The process comprises identifying one or more receptors expressed in testicular tissue or germ cells and identifying ligands of the identified receptors. The process also comprises identifying one or more proliferation factors that improve the level of proliferation of testicular germ cells. Identifying one or more proliferation factors comprises culturing testicular tissue in vitro in one or more second culture media, wherein the one or more second culture media comprise the first culture medium supplemented with one or more factors that regulate an identified biological pathway. The second cultures that support improved levels of germ cell proliferation comprise one or more proliferation factors, thereby identifying the one or more factors that improve the level of proliferation of testicular germ cells. Culture media can be as described in Section II herein below and culturing testicular tissue comprising testicular germ cells or isolated testicular germ cells can be as described in Section III herein below.

In some aspects, the process of identifying receptors and receptor ligands, screening the ligands, or any combination thereof can be iteratively repeated to identify proliferation factors to identify additional factors, various combinations of identified factors, various levels of the factors, and any combination thereof that improve the level of proliferation of testicular germ cells.

Yet another aspect of the instant disclosure encompasses an iterative process for identifying culture conditions supportive of testicular germ cell proliferation in vitro. The process comprises (1) identifying the receptors from a database of receptors, receptors specifically expressed in testicular identified using scRNA-seq transcriptional profiles of testicular tissue or testicular germ cells, receptors specifically expressed in testicular identified using scRNA-seq transcriptional profiles of testicular tissue or testicular germ cells; (2) identifying ligands of the identified receptors; (3) culturing testicular germ cells or testicular tissue in vitro in a culture medium supplemented with one or more of the identified ligands; (4) identifying culture media that support improved levels of germ cell proliferation when compared to the level of germ cell proliferation in un-supplemented culture medium, thereby identifying the one or more factors that improve the level of proliferation of testicular germ cells. The process further comprises iteratively repeating the process of identifying receptors and receptor ligands, screening the ligands, or any combination thereof can be iteratively repeated to identify proliferation factors to identify additional factors, various combinations of identified factors, various levels of the factors, and any combination thereof that improve the level of proliferation of testicular germ cells. The culture media that support testicular germ cell proliferation comprise one or more of the identified ligands.

Identifying the one or more receptors expressed in testicular tissue or germ cells comprises selecting about 150 receptors comprising the highest level of expression in the testicular cells, ranking the receptors by expression level in decreasing order, and excluding receptors that have ubiquitous expression in testicular tissue, i.e., expressed in testicular cells outside of SSCs, developing spermatogonia, or both. Identifying ligands of the identified receptors comprises identifying from a database of ligands, ligands expressed in human testes. The database of receptors and receptor ligands is CellTalkDB.

The process also comprises identifying one or more proliferation factors that improve the level of proliferation of testicular germ cells. Identifying one or more proliferation factors comprises culturing testicular tissue in vitro in one or more second culture media, wherein the one or more second culture media comprise the first culture medium supplemented with one or more factors that regulate an identified biological pathway. The second cultures that support improved levels of germ cell proliferation comprise one or more proliferation factors, thereby identifying the one or more factors that improve the level of proliferation of testicular germ cells. Culture media can be as described in Section II herein below and culturing testicular tissue comprising testicular germ cells or isolated testicular germ cells can be as described in Section III herein below.

Using the process of the instant disclosure, the inventors were able to identify receptors, receptor ligands, and the ligands among them that can improve proliferation of SSCs and differentiating spermatogonia. In some aspects, ligands of the identified receptors can be as described in Section II herein below. In some aspects, the identified receptors specifically expressed in SSCs and differentiating spermatogonia, the ligands of the receptors, and ligands that can improve proliferation of germ cells can be as listed in Table 5.

### II. Culture Media

Another aspect of the instant disclosure encompasses a culture medium supportive of testicular germ cell proliferation in vitro. The terms “medium”, “media”, “culture medium”, or “culture media,” as used herein, refers to an aqueous based solution that is provided for the growth, viability, or storage of cells used in carrying out the present invention. A “base media,” as used herein, refers to a basal salt nutrient or an aqueous solution of salts and other elements that provide cells with water and certain bulk inorganic ions essential for normal cell metabolism and maintains intracellular and/or extra-cellular osmotic balance. Base media can comprise energy sources such as glucose or galactose, amino acids, vitamins, salts, a buffering system to maintain the medium within the physiological pH range, or any combination thereof. Base media for culturing mammalian cells are known in the art and can be available commercially. Non-limiting examples of base media include phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5A Medium, Medium 199, William's Medium E, insect media such as Grace's medium, Ham's Nutrient mixture F-10 (Ham's F-10), Ham's F-12, α-Minimal Essential Medium (αMEM), Glasgow's Minimal Essential Medium (G-MEM) and Iscove's Modified Dulbecco's Medium.

In the context of testicular cells, certain specialized media have been developed. For instance, F12/DMEM, often supplemented with fetal bovine serum (FBS), is commonly used. Other media, like M199, can also be utilized. In addition, specific supplements may be included depending on the cell type. For instance, for the culture of Sertoli cells (a type of testicular cell), supplements can include insulin, transferrin, and biotin, among others. Furthermore, in some instances, growth factors such as glial cell line-derived neurotrophic factor (GDNF) and fibroblast growth factor (FGF) might be used to support spermatogonial stem cell cultures. However, the specific formulation can and will vary widely depending on the specific requirements of the testicular cells in question. In some aspects, the culture medium further comprises components that support the proliferation of stem cells in culture. Such a medium can be obtained commercially, e.g., STEMPRO-34, Pluripotent Stem Cell SFM XF/FF, or derived from a stem cell culture medium known in the art. In addition to a basal medium, the culture medium can include additional components or supplements to support the proliferation of testicular cells Such supplements include, but are not limited to, fetal bovine serum, Knockout Serum Replacement (KSR), B27 supplement, glial cell-derived neurotrophic factor (GDNF), basic fibroblast growth factor (bFGF), epidermal growth factor (EGF), stem cell factor (SCF) retinoic acid, follicle-stimulating hormone (FSH), an antibiotic, an antifungal, and the like.

In some aspects, the culture medium comprises a base medium supplemented with proliferation factors that improve the level of proliferation of testicular germ cells in vitro. In some aspects, a base culture medium is αMEM supplemented with KSR. In some aspects, a base culture medium is αMEM+10% KSR.

In some aspects, the culture medium comprises base medium supplemented with proliferation factors previously identified using a process of the instant disclosure. A process of the instant disclosure can be as described in Section I herein above. Proliferation factors previously identified using a process of the instant disclosure can be as described herein.

In some aspects, a medium of the instant disclosure comprises a culture medium supplemented with proliferation factors identified by identifying one or more dysregulated pathways in cells of cultured testicular tissue, identifying factors that can regulate the dysregulated pathways (referred to hereinafter as pathway factors), and identifying among the pathway factors, factors that improve the level of proliferation of testicular germ cells by screening the identified pathway factors for factors that can regulate the identified dysregulated pathways to identify the factors among them that can improve germ cell proliferation. In some aspects, the pathway factors can be identified as described in Section I(b), and the proliferation factors can be as identified in Section I(d).

In some aspects, a medium of the instant disclosure is supplemented with one or more of an inhibitor of hypoxia-inducible factor (HIF), an anti-apoptosis factor, an anti-inflammation factor, a ROS inhibitor, a gonadocorticoid, a gonadotropin, a member of the GDNF family of ligands (GFL), an activin, a fibroblast growth factor receptor (FGFR) protein ligand, an interleukin 6 cytokine, a chemokine, a retinoic acid receptor ligand, a ligand of receptor of Table 5, or any combination thereof. In some aspects, HIF can be HIF-1a, VHL E3 ubiquitin ligase (VHL), or a combination thereof. In some aspects, HIF-1a inhibitor can be a polyamide (disrupts the HIF-1-DNA interface), acriflavine (inhibits dimerization of HIF-1), chetomin (disruptes the HIF-1-p300 interaction), YC1 (inactivates the transcriptional activity of HIF-1a), amphotericin B (inactivates the transcriptional activity of HIF-1a), AJM290 (inactivates the transcriptional activity of HIF-1α), AW464 (inactivates the transcriptional activity of HIF-1α), PX-12 (inhibits HIF-1α protein levels), PX-478 (inhibits HIF-1α protein levels), aminoflavone (inhibits HIF-1α protein levels), EZN-2968 (an RNA antagonist of HIF1α), echinomycin (disrupts the HIF-1-DNA interface), or any combination thereof.

In some aspects, proliferation factors are selected from Testosterone, Activin A, FSH, GDNF, FGF2, LIF, RA, CXCL12, WNT-3A, Neurturin (NRTN), Netrin-1 (NTN1), BMP2, rh beta-NGF, rh Midkine Protein, rh HB-EGF, rh Holo-Transferrin, rh MIF, rh CXCL4, rh LIF, insulin, proliferation factors listed in Table 12, and any combination thereof.

In some aspects, a proliferation factor of the instant disclosure is a HIF-1α inhibitor. In some aspects, the HIF-1α inhibitor is echinomycin, PX-12, vitexin, or any combination thereof. In one aspect, the HIF-1α inhibitor is echinomycin, and the concentration of echinomycin in the culture media can range from about 0.1 nM to about 100 nM, about 1 nM to about 50 nM, or about 2 nM to about 7 nM.

In some aspects, a proliferation factor of the instant disclosure is a gonadocorticoid. In some aspects, the gonadocorticoid can be an androgen. The androgen can be testosterone, FSH, hCG, LH, GDNF, or a combination thereof. In some aspects, the androgen is testosterone, and the concentration of testosterone in the culture media ranges from about 10⁻⁵M to about 10⁻⁹M, from about from about 10⁻⁶M to about 10⁻⁸M, or from about 1.5×10⁻⁶M to about 0.5×10⁻⁸M. In some aspects, the androgen is testosterone, and the concentration of testosterone in the culture media ranges from about 8 uM to about 150 uM, from about 18 uM to about 140 uM, from about 28 uM to about 130 uM, from about 38 uM to about 120 uM, from about 48 uM to about 110 uM, from about 58 uM to about 110 uM, from about 68 uM to about 110 uM, or from about 78 uM to about 110 uM. In some aspects, the androgen is testosterone, and the concentration of testosterone in the culture media ranges from about 78 uM to about 82 uM.

In some aspects, a proliferation factor of the instant disclosure is a member of GFL. In some aspects, the member of GFL can be GDNF. In some aspects, the concentration of GDNF in the culture media ranges from about 0.1 ng/mL to about 100 ng/mL, about 1 ng/mL to about 50 ng/mL, or about 7 ng/mL to about 12 ng/mL.

In some aspects, a proliferation factor of the instant disclosure is a fibroblast growth factor receptor (FGFR) protein ligand. In some aspects, the FGFR protein ligand can be bFGF (FGF2). In some aspects, the concentration of bFGF in the culture media ranges from about 0.1 ng/mL to about 100 ng/mL, about 1 ng/mL to about 50 ng/mL, or about 7 ng/mL to about 12 ng/mL.

In some aspects, a proliferation factor of the instant disclosure is a gonadotropin. The gonadotropin can be human chorionic gonadotropin (hCG), leutenizing hormone (LH), or both. The activin can be activin A. The concentration of activin A in the culture media can range from about 0.1 ng/mL to about 200 ng/mL, about 1 ng/mL to about 150 ng/mL, or about 25 ng/mL to about 75 ng/mL.

In some aspects, a proliferation factor of the instant disclosure is an interleukin 6 cytokine. The interleukin 6 cytokine can be leukemia inhibitory factor (LIF). In some aspects, the concentration of LIF in the culture media ranges from about 1 ng/mL to about 500 ng/mL, about 10 ng/mL to about 200 ng/mL, or about 75 ng/mL to about 125 ng/mL.

In some aspects, a proliferation factor of the instant disclosure is a chemokine. The chemokine can be CXCL12. In some aspects, the concentration of CXCL12 in the culture media ranges from about 1 ng/mL to about 500 ng/mL, about 10 ng/mL to about 200 ng/mL, or about 75 ng/mL to about 125 ng/mL.

In some aspects, a proliferation factor of the instant disclosure is a retinoic acid. The retinoic acid receptor ligand can be retinoic acid. In some aspects, the concentration of retinoic acid in the culture media ranges from about 10⁻⁵M to about 10⁻⁹M, from about from about 10⁻⁶M to about 10⁻⁸M, or from about 2.5×10⁻⁷M to about 3.5×10⁻⁷M.

In some aspects, a proliferation factor of the instant disclosure is insulin. In some aspects, the concentration of insulin in the culture media ranges from about 0.1 ng/mL to about 100 ng/mL, about 1 ng/mL to about 50 ng/mL, about 7 ng/mL to about 12 ng/mL, or about 9 ng/mL to about 11 ng/mL.

In some aspects, a medium of the instant disclosure comprises αMEM with KSR base medium supplemented with one or more factors selected from echinomycin, testosterone, RA, and FSH and any combination thereof. In other aspects, a medium of the instant disclosure comprises αMEM with KSR base medium supplemented with one or more factors selected from echinomycin, testosterone, and GDNF. In yet other aspects, a medium of the instant disclosure comprises αMEM with KSR base medium supplemented with one or more factors selected from echinomycin, testosterone, GDNF, HCG, and FSH. In some aspects, a medium of the instant disclosure comprises αMEM with KSR base medium supplemented with one or more factors selected from Testosterone, Activin A, FSH, GDNF, FGF2, LIF, RA, CXCL12, and any combination thereof.

In some aspects, a culture medium of the instant disclosure comprises, Testosterone, GDNF, and FGF2. In some aspects, a culture medium of the instant disclosure comprises αMEM+10% KSR, echinomycin at a concentration ranging from about 4 nM to about 6 nM, Testosterone at a concentration ranging from about 10⁻⁶ M to about 10⁻⁸ M, GDNF at a concentration ranging from about 9 ng/ml to about 11 ng/mL, and FGF2 at a concentration ranging from about 9 ng/ml to about 11 ng/mL.

In some aspects, a culture medium of the instant disclosure comprises αMEM+10% KSR, Penicillin-Streptomycin, GDNF, FGF2, Insulin, EGF, Testosterone, and Echinomycin. In some aspects, a culture medium of the instant disclosure comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, Testosterone at a concentration ranging from about 9 uM to about 11 uM, and Echinomycin at a concentration ranging from about 4 ng/ml to about 6 ng/ml. (Condition 2)

In some aspects, a culture medium of the instant disclosure comprises αMEM+10% KSR, Penicillin-Streptomycin, GDNF, FGF2, Insulin, EGF, Testosterone, and Echinomycin. In some aspects, a culture medium of the instant disclosure comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, and Testosterone at a concentration ranging from about 9 uM to about 11 uM. (C2) In some aspects, a culture medium of the instant disclosure comprises αMEM+10% KSR, Penicillin-Streptomycin at a concentration ranging from about 0.9% to about 1.1%, GDNF at a concentration ranging from about 19 ng/ml to about 21 ng/ml, FGF2 at a concentration ranging from about 19 ng/ml to about 21 ng/ml, Insulin at a concentration ranging from about 9 ug/ml to about 11 ug/ml, EGF at a concentration ranging from about 19 ng/ml to about 21 ng/ml, and Testosterone at a concentration ranging from about 78 uM to about 82 uM. (C2 with eight times testosterone)

In some aspects, media comprising proliferation factors can be further supplemented with antioxidant molecules. In some aspects, the antioxidant molecules comprise GSH, VC, VA, or any combination thereof. In some aspects, the antioxidant molecules comprise GSH, VC, and VA.

### III. Cell Cultures

An additional aspect of the instant disclosure encompasses a testicular cell culture for culturing testicular germ cells in vitro. The cell culture comprises testicular tissue comprising germ cells or testicular germ cells in a culture medium comprising proliferation factors. The testicular tissue comprising germ cells or testicular germ cells can be as described in Section I(a) herein above. The culture medium and proliferation factors can be as described in Section II herein above. In some aspects, the factors that improve the level of proliferation of testicular germ cells in vitro can be identified using any of the processes described in Section I herein above.

In some aspects, the testicular cell culture comprises testicular tissue comprising testicular germ cells. In some aspects, the testicular tissue comprises seminiferous tubules. In some aspects, the testicular cell culture comprises isolated testicular germ cells. In some aspects, the testicular cell culture comprises isolated spermatogonia. In some aspects, the tissue culture comprises C2 media. In some aspects, the tissue culture comprises Control 2 media.

Importantly, the inventors surprisingly discovered that to successfully culture germ cells, methods of preparing testicular tissue differ from methods of preparing testicular germ cells for culture. For instance, the inventors developed a method of preparing testicular tissue for successfully culturing testicular germ cells. In some aspects, testicular tissue comprising testicular germ cells is prepared as described in Example 2 herein below. The inventors also developed a method of preparing testicular germ cells for successful proliferation. In some aspects, the spermatogonia are isolated as described in Example 6 herein below.

Equally importantly, the inventors also discovered that suitable media for successful culturing germ cells differ from methods of preparing testicular germ cells for culture. For instance, control 2 media was found to be suitable for culturing testicular tissue, whereas C2 media was better at culturing isolated testicular germ cells. Accordingly, in some aspects, a testicular cell culture of the instant disclosure can comprise control 2 media and testicular tissue comprising testicular germ cells. In some aspects, a testicular cell culture of the instant disclosure can comprise control 2 media comprising eight times testosterone and testicular tissue comprising testicular germ cells. This notable because to date, such an elevated concentration of testosterone has not been described to be effective or useful for culturing testicular tissue, testicular germ cells, or any other tissue. In other aspects, a testicular cell culture of the instant disclosure comprises C2 media and isolated testicular germ cells. Accordingly, in some aspects, a testicular cell culture of the instant disclosure can comprise control 2 media and testicular tissue comprising testicular germ cells, wherein the tissue is isolated using a method described in Example 2. Accordingly, in some aspects, a testicular cell culture of the instant disclosure can comprise control 2 media comprising eight times testosterone and testicular tissue comprising testicular germ cells, wherein the tissue is isolated using a method described in Example 2. In other aspects, a testicular cell culture of the instant disclosure comprises C2 media and isolated testicular germ cells, wherein the germ cells are isolated using a method described in Example 6.

### IV. Method of In Vitro Culture

Another aspect of the instant disclosure encompasses a method of culturing testicular germ cells in vitro. The method comprises culturing testicular tissue comprising germ cells or isolated germ cells in culture media comprising proliferation factors. In some aspects, the method of culturing can further comprise preparing testicular tissue for culture or isolating testicular germ cells for culture. Proliferation factors can be factors identified using a process described in Section I herein above. In some aspects, proliferation factors and proliferation media can be as described in Section II herein above. The testicular tissue and testicular germ cells can be as described in Section I(a) herein above. Preparing testicular tissue and isolating testicular germ cells can be as described in Section III.

Importantly, despite the essential role of the testis niche in spermatogenesis, a process of the instant disclosure was able to identify culturing isolated testicular germ cells independent of other testicular tissue to identify culture conditions supportive of testicular germ cell proliferation in vitro. This is in part due to the discovery of germ cell proliferation factors identified using a process of the instant disclosure that permit proliferation of the isolated germ cells in vitro independent of the testis niche. The inventors surprisingly discovered that the process of the instant disclosure can identify culture conditions that allows germ cell culture where the germ cells maintain their identity, growth, survival, and replication of the germ cells for extended periods of time. For instance, germ cells can be cultured in the identified culture conditions for 1 week or longer, 2 weeks or longer, 3 weeks or longer, 1 month or longer, 2 months or longer, 1 year or longer, or indefinitely. In some aspects, germ cells can be cultured in the identified culture conditions for 14 days or longer. In some aspects, germ cells can be cultured in the identified culture conditions for 21 days or longer. In some aspects, germ cells can be cultured in the identified culture conditions for 28 days or longer.

In some aspects, a culture method of the instant disclosure comprises culturing testicular germ cells in a culture comprising testicular tissue for a period of time, followed by isolating the testicular germ cells in the cultured tissue and culturing the isolated testicular germ cells independently of the tissue. In some aspects, such a hybrid method of culturing testicular germ cells can be used to culture the germ cells for 28 days and longer. In some aspects, such a hybrid method of culturing testicular germ cells can be as described in Example 6.

As explained herein above, the inventors surprisingly discovered that for successfully culturing germ cells, methods of preparing testicular tissue differ from methods of preparing testicular germ cells for culture. For instance, the inventors developed a method of preparing testicular tissue for successfully culturing testicular germ cells. In some aspects, testicular tissue comprising testicular germ cells is prepared as described in Example 2 herein below. Accordingly, when a method of culturing testicular germ cells of the instant disclosure comprises culturing testicular tissue, the method can comprise preparing the testicular tissue using the developed method of preparing testicular tissue. In some aspects, when a method of culturing testicular germ cells of the instant disclosure comprises culturing testicular tissue, the method comprises preparing the testicular tissue using a method of preparing testicular tissue described in Example 2 herein below.

The inventors also developed a method of preparing testicular germ cells for successful proliferation. In some aspects, the spermatogonia are isolated as described in Example 6 herein below. Accordingly, when a method of culturing testicular germ cells of the instant disclosure comprises culturing isolated testicular germ cells, the method can comprise preparing the germ cells using the developed method of preparing testicular tissue. In some aspects, when a method of culturing testicular germ cells of the instant disclosure comprises culturing isolated testicular germ cells, the method comprises preparing the testicular tissue using a method of preparing isolated testicular germ cells described in Example 6 herein below.

Yet another aspect of the present disclosure encompasses a method of obtaining spermatozoa from fertile and infertile men through culturing. The method comprises culturing more than one spermatogonial stem cell using culture conditions identified using the process described in Section I herein above. Each SSC is separately cultured. The method further comprises identifying a spermatogonial stem cell culture comprising sperm produced by the cultured SSC. The sperm do not contain deleterious heritable mutations and/or contain lower rates of de novo mutations, and comprise an expressed RNA transcript profile substantially similar to the expressed RNA transcript profile of the SSC in the SSC culture. Additionally, the method comprises harvesting spermatozoa from the identified culture conditions. The method can further comprise the step of freezing the spermatozoa for future use. In some aspects, the method further comprises the step of using the spermatozoa with assisted reproductive technologies such as intrauterine insemination or in vitro fertilization.

One aspect of the present disclosure encompasses a method of producing viable spermatozoa. The method comprises obtaining or having obtained testicular tissue from a subject; and culturing the testicular tissue in culture conditions identified using the process described in Section I herein above, the testicular cell culturing system described herein above, or both.

### V. Kits

A further aspect of the present disclosure encompasses a kit for culturing testicular germ cells in vitro under conditions identified using the process described in Section I herein above, the testicular cell culturing system described herein above, or both.

Kits according to the present disclosure can include one or more additional reagents useful for culturing testicular tissue and germ cells according to the present disclosure. A kit generally includes a package with one or more containers holding the reagents, as one or more separate compositions or, optionally, as admixture where the compatibility of the reagents will allow. The test kit can also include other material(s), which may be desirable from a user standpoint, such as a buffer(s), a diluent(s), a standard(s), and/or any other material useful in processing or conducting any other step of the tagging method.

Kits according to the present disclosure preferably include instructions for culturing testicular tissue and germ cells. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

“Mammalian,” as used herein refers to both human subjects (and cells sources) and non-human subjects (and cell sources or types), such as dog, cat, mouse, monkey, etc. (e.g., for veterinary purposes).

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Single-Cell Analysis of the Developing Human Testis

Human testis development in prenatal life involves complex changes in germline and somatic cell identity. To better understand, 32,500 single-cell transcriptomes of testicular cells from embryonic, fetal, and infant stages were profiled and analyzed. The results show that at 6-7 weeks postfertilization, as the testicular cords are established, the Sertoli and interstitial cells originate from a common heterogeneous progenitor pool, which then resolves into fetal Sertoli cells (expressing tube-forming genes) or interstitial cells (including Leydig-lineage cells expressing steroidogenesis genes). Almost 10 weeks later, beginning at 14-16 weeks post-fertilization, the male primordial germ cells exit mitosis, downregulate pluripotent transcription factors, and transition into cells that strongly resemble the state 0 spermatogonia originally defined in the infant and adult testes. Therefore, these fetal spermatogonia were called “state f0.” Overall, multiple insights into the coordinated and temporal development of the embryonic, fetal, and postnatal male germline together with the somatic niche were reveal.

Within the developing fetal testicular niche, recent genomics profiling and immunofluorescence (IF) imaging approaches have revealed that male germline cells undergo major developmental changes (Gkountela et al., 2013, 2015; Guo et al., 2015; Li et al., 2017; Tang et al., 2015). Notably, the germline transitions from pluripotent-like PGCs migrating to and into the developing gonad to pluripotent-like and mitotically active PGCs in the gonad (called fetal germ cells [FGCs] or gonocytes), followed by the transition to “mitotically arrested” germ cells that repress the pluripotency-like program at/after weeks 14-18 (Li et al., 2017). Here, a key unanswered question during this stage of germline development involving the relationship between the mitotically arrested germ cells that arise during weeks 14-18 and the postnatal SSCs is as follows: are prenatal germ cells nearly identical to postnatal SSCs or are there major additional developmental stages that occur during prenatal stages? Notably, prior work by the inventors on the adult testis identified five distinct spermatogonial states (called states 0-4) accompanying human spermatogonial differentiation, with state 0 identified as the most naive and undifferentiated state (Guo et al., 2017, 2018, 2020), a result supported by single-cell RNA sequencing (scRNA-seq) profiling from other groups (Hermann et al., 2018; Li et al., 2017; Shami et al., 2020; Sohni et al., 2019; Wang et al., 2018). Consistent with this notion, state 0 is the predominant SSC state present in the infant testis, and state 0 SSCs express hundreds of state-specific markers, including PIWIL4, TSPAN33, MSL3, and EGR4 (Guo et al., 2018). The key markers identified in state 0 SSCs are also expressed in the undifferentiated spermatogonial states identified by others in recent studies, such as the SSC1-B (Sohni et al., 2019) or SPG-1 adult spermatogonia population (Shami et al., 2020), as well as in spermatogonia profiled from human neonates (Sohni et al., 2019) and in undifferentiated spermatogonia from macaques (Shami et al., 2020). Here, it is explored whether the previously identified mitotically arrested prenatal germ cells transcriptionally resemble state 0 postnatal spermatogonia, or instead represent a unique precursor that undergoes additional prenatal changes before birth.

The testis niche plays an important role in guiding the survival and differentiation of the male germline. In the adult testis, somatic niche cells, including Sertoli, Leydig, and myoid cells, provide physical and hormonal support for the successful execution of spermatogenesis from SSCs (Guo et al., 2018). The development of the functional adult testis and its organized tubule-like structure is completed at puberty, during which the final specification and maturation of all somatic niche cells takes place. Prior work by the inventors, which used scRNA-seq to study human testis development during puberty, revealed a common progenitor for Ley-dig and myoid cells that exists before puberty in humans, which is analogous to the somatic progenitor observed in fetal mice (Guo et al., 2020). However, during prenatal life, several key issues remain elusive, such as how the human testicular niche cell lineages are initially specified, whether they have a common progenitor, how the nascent gonad initially forms cords, and how niche cells differentiate further during subsequent fetal developmental stages to arrive at their postnatal states.

To address these questions, a total of 32,500 unsorted single testicular cells from embryonic, fetal, and postnatal samples were profiled through the 10× Genomics Chromium platform. This unbiased profiling allowed us to examine the specification process in the somatic cell niche and the development of both the germline and niche cells; this enabled a detailed comparison of the cell types and developmental processes in infant, pubertal, and adult testis.

### Results

### Single-Cell Transcriptomes of Human Embryonic, Fetal, and Postnatal Testes

Human testis tissues were obtained from 3 embryonic stages (6, 7, and 8 weeks postfertilization), 3 fetal stages (12, 15, and 16 weeks postfertilization), and 1 young infant stage (5 months postbirth) for comparisons to prior datasets from older infants, juveniles, and adults. To systematically investigate both germ cell and somatic cell development across embryonic and fetal stages, single-cell suspensions were prepared from these testicular tissues and performed scRNA-seq using the 10× Genomics platform. For embryonic and fetal samples, 5,000 single cells per sample were profiled; for the young infant sample, 2 replicates were performed, and profiled 2,500 single cells. From a total of 32,500 cells, 30,045 passed standard quality control dataset filters and were retained for downstream analysis (see Method details). 80,000-120,000 reads/cell were obtained, which enabled the analysis of 1,800-2,500 genes/cell.

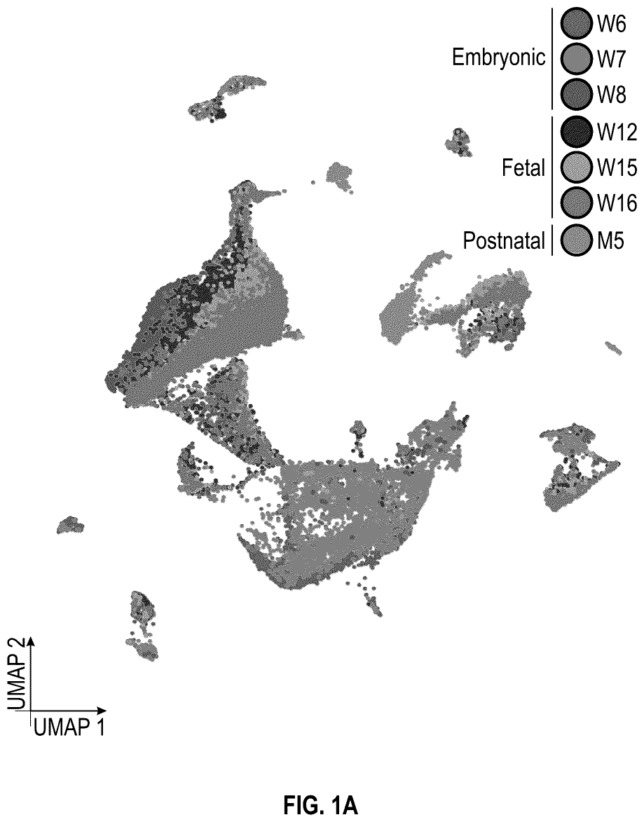

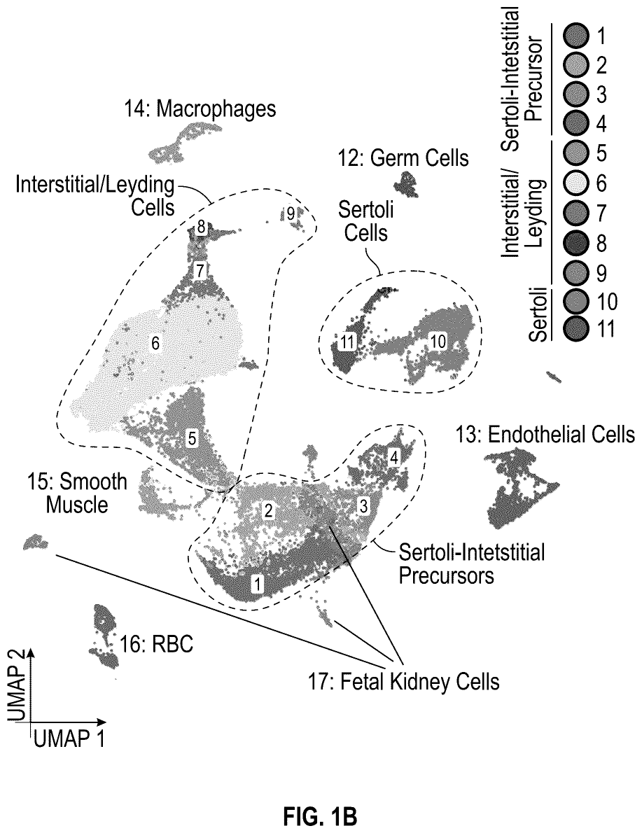

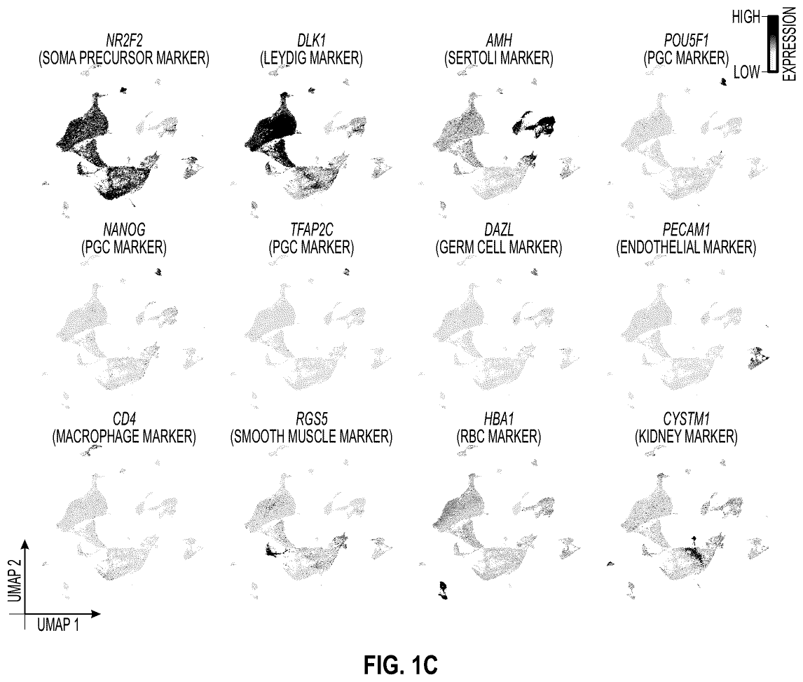

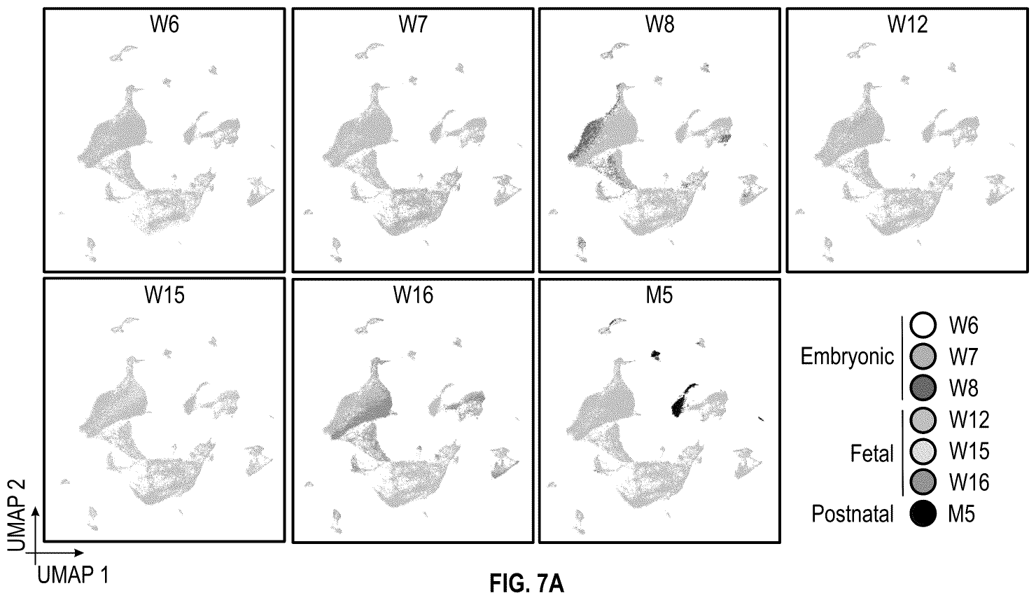

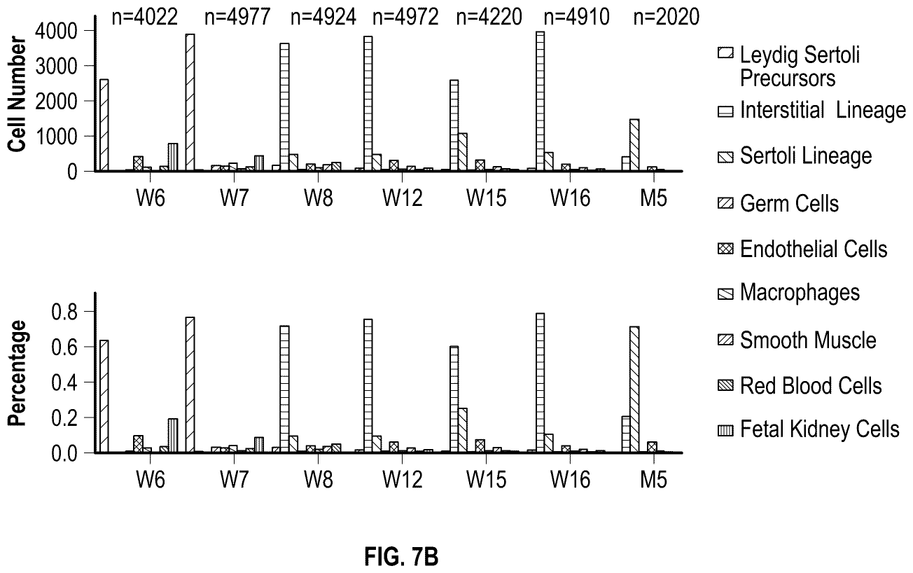

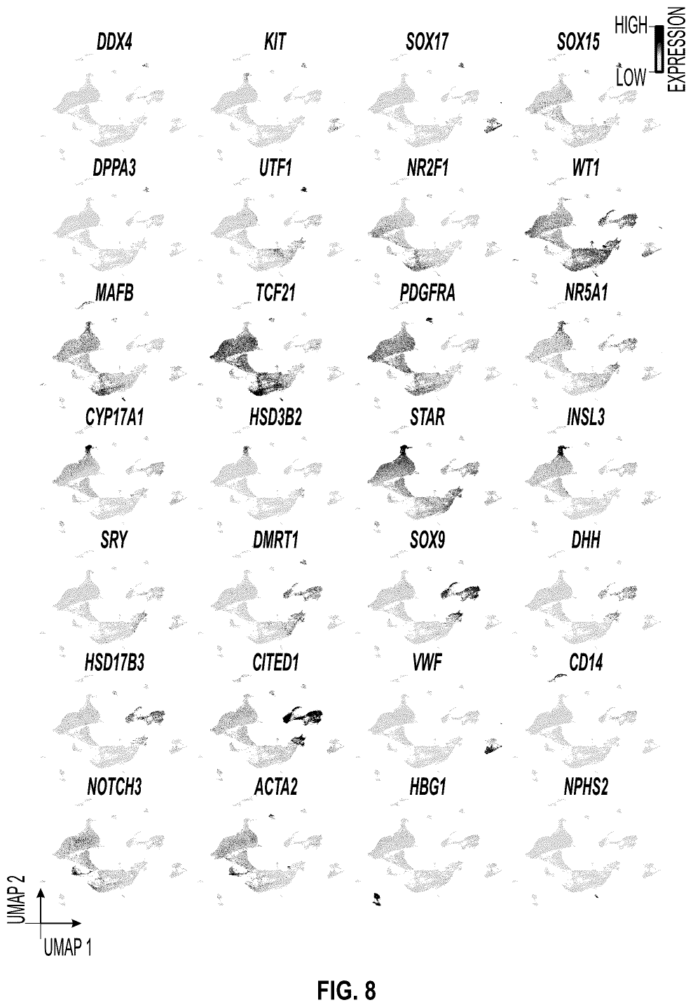

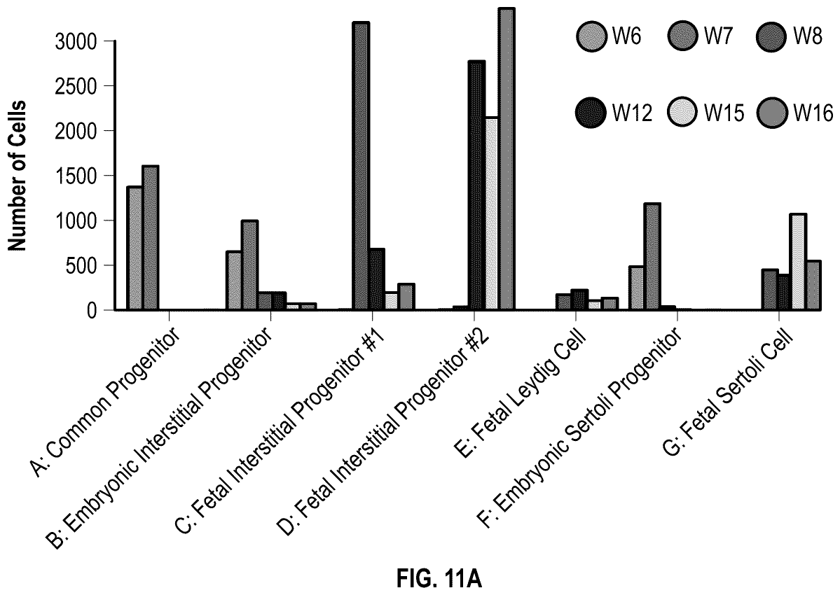

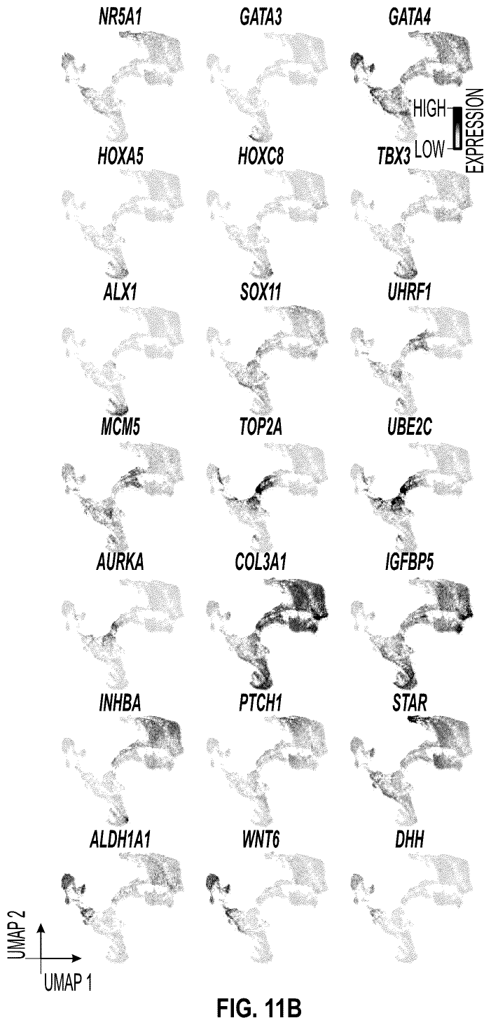

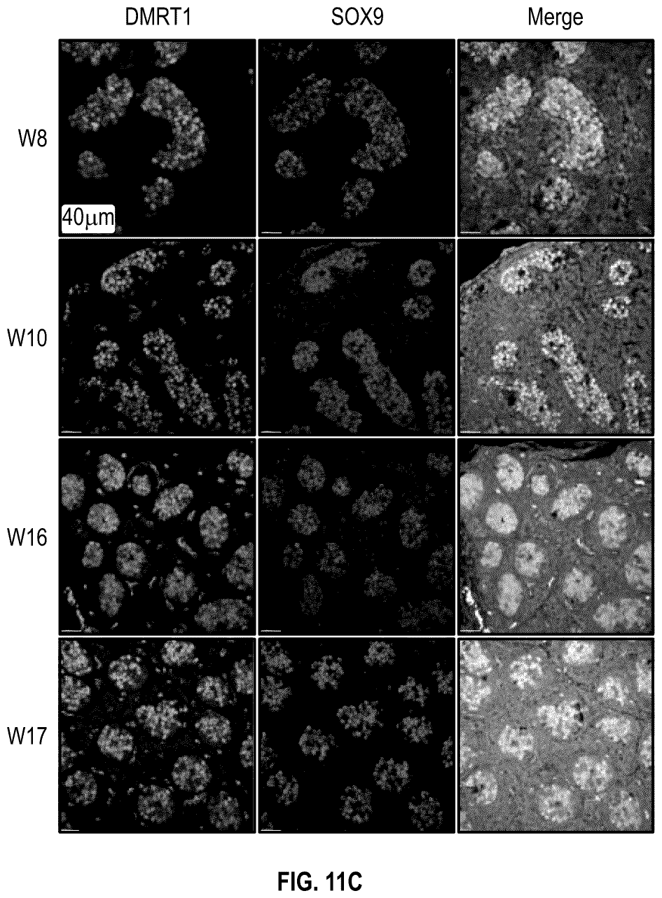

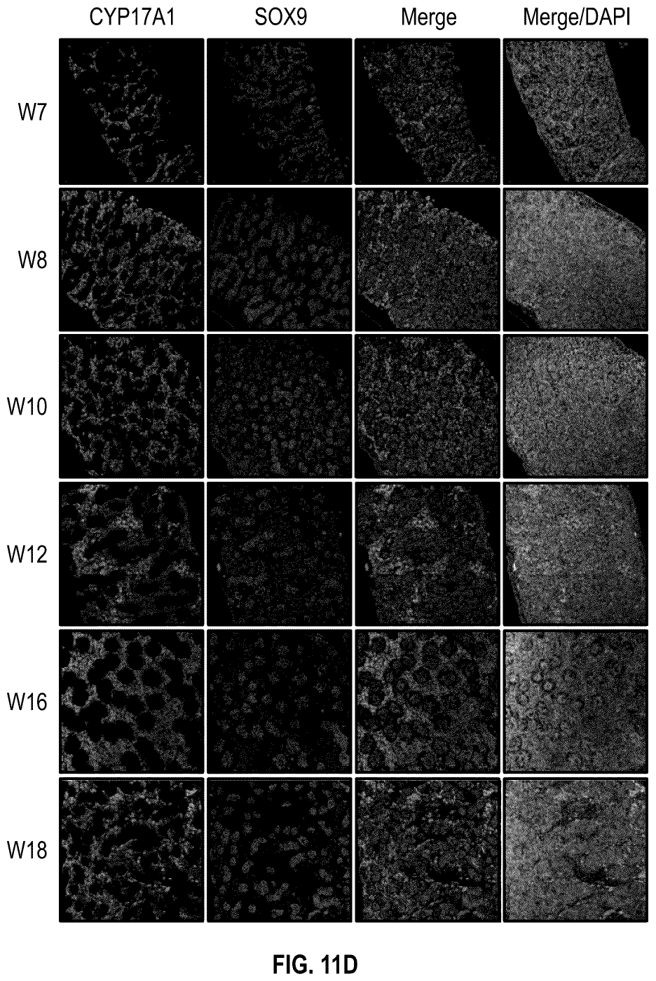

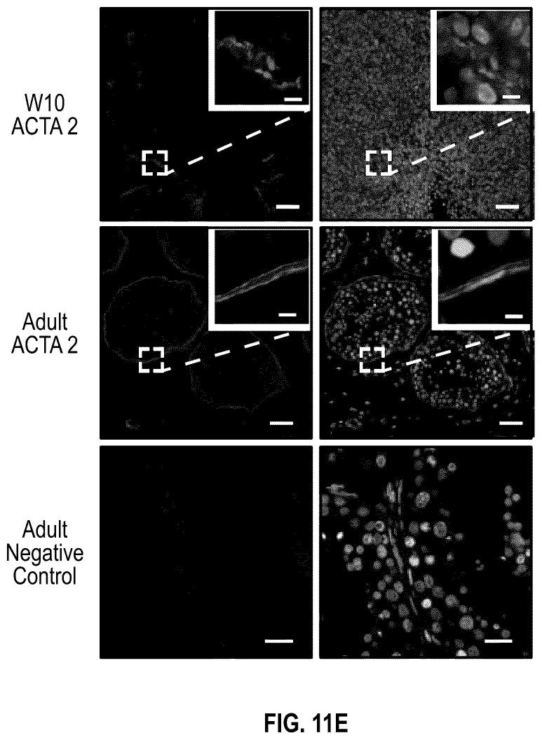

To analyze the dataset, UMAP (uniform manifold approximation and projection dimension reduction analysis) was first performed on the combined datasets using the Seurat package (FIG. 1A and FIG. 7A: Butler et al., 2018). Interestingly, a trend was observed in which cells from 6 and 7 weeks cluster closely, and likewise, cells from 8, 12, 15, and 16 weeks cluster closely (FIG. 1A and FIG. 7A), while also displaying temporal changes in particular cell types (FIG. 7B and FIG. 7C). Further clustering analyses yielded 17 major clusters or cell types (FIG. 11B) that were subsequently annotated using known gene markers (FIG. 1C and FIG. 8). Clusters 1-4 are testicular niche cells from 6- and 7-week embryos, which uniquely express NR2F2 and TCF21. Clusters 5-9 correspond to somatic cells from the interstitial and Leydig lineage from 8-week samples, which express DLK1. Clusters 10-11 are Sertoli lineage cells from 8-week samples, which express AMH and SOX9. Cluster 12 includes germ cells from all of the samples, which express known germ cell markers (e.g., TFAP2C, DAZL) with a subset expressing markers of pluripotency (e.g., POU5F1, NANOG). Clusters 13-17 correspond to endothelial cells (cluster 13, PECAM1⁺), macrophages (cluster 14, CD4⁺), smooth muscle cells (cluster 15, RGS5⁺), red blood cells (cluster 16, HBA1⁺), and fetal kidney cells (cluster 17, CYSTM1⁺), respectively. Examples of the many additional markers that were used to define these cell types were also provide (FIG. 8).

### Emergence of State 0 SSCs as PGCs Exit Mitosis and Repress Pluripotency

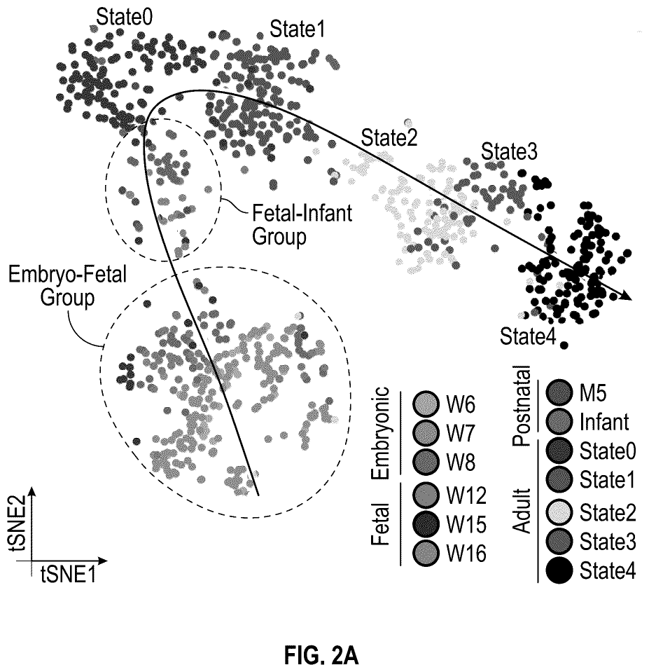

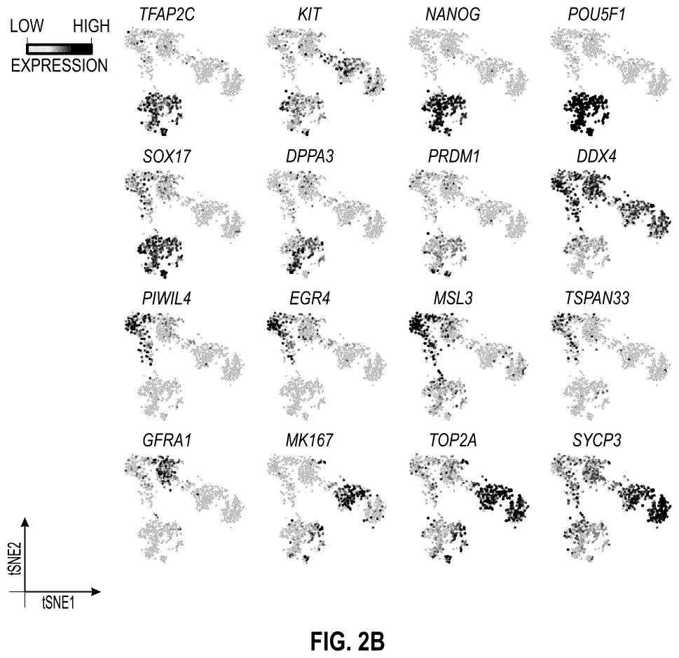

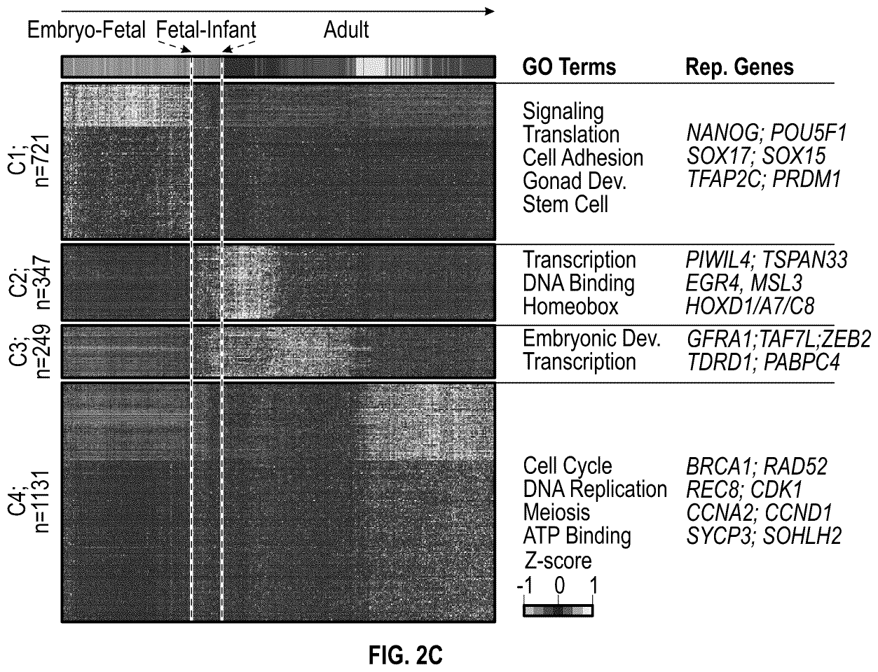

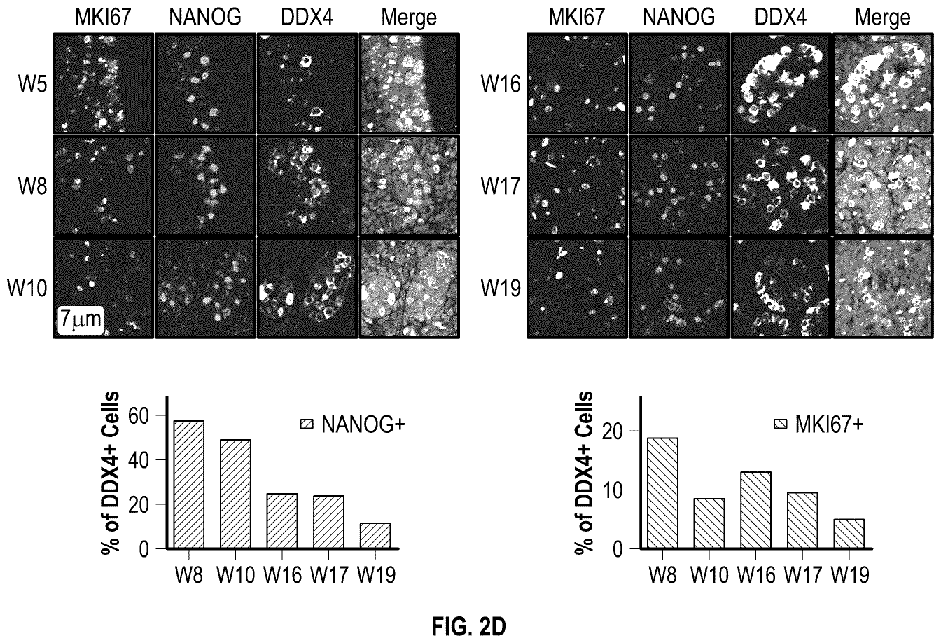

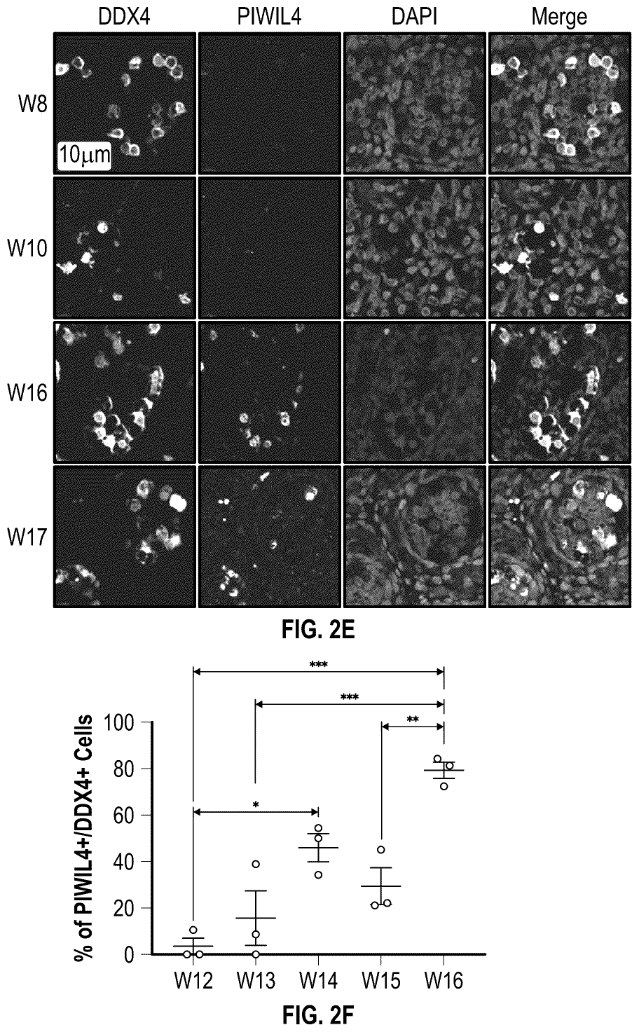

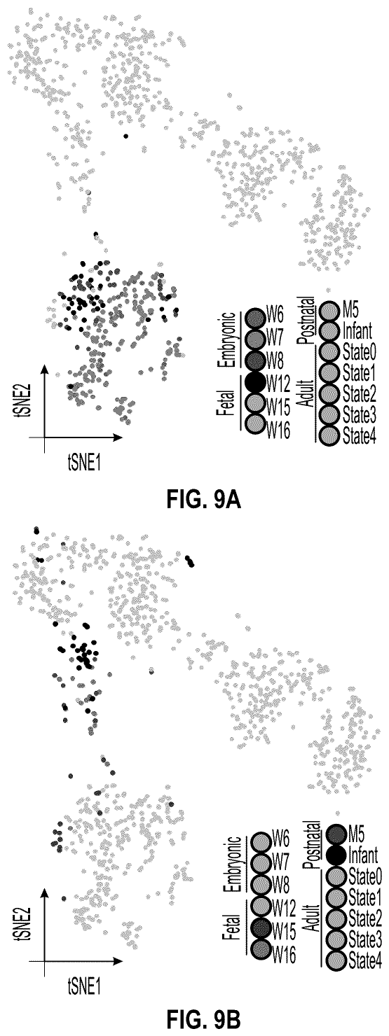

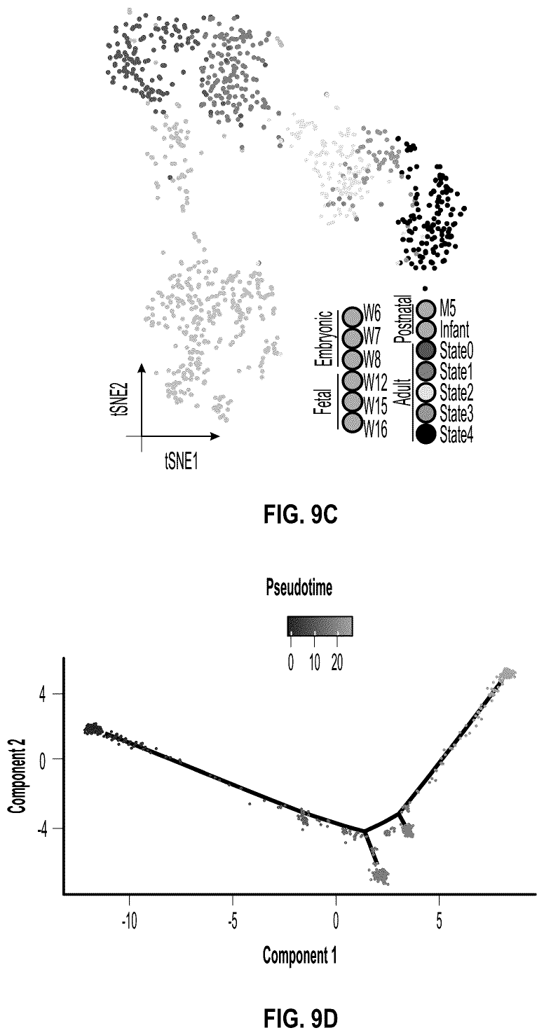

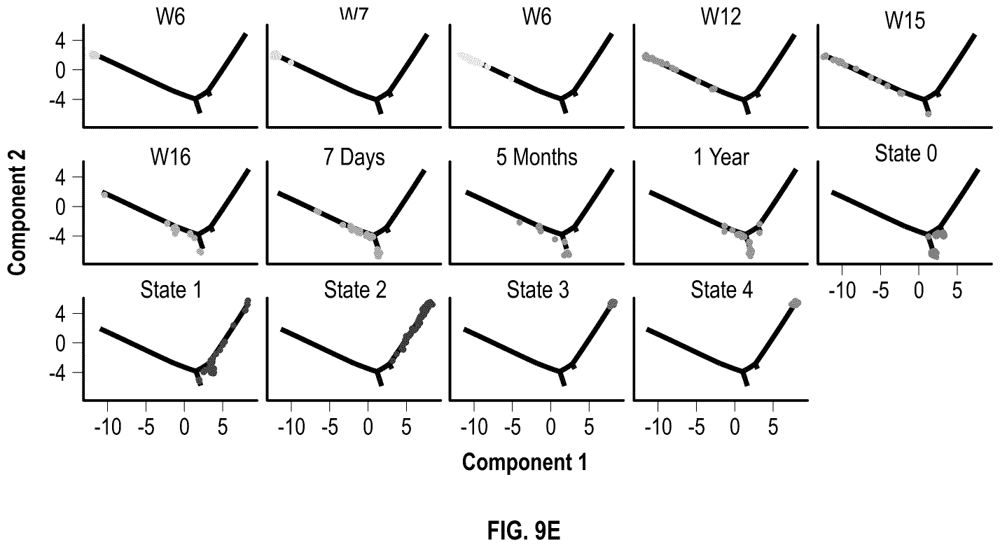

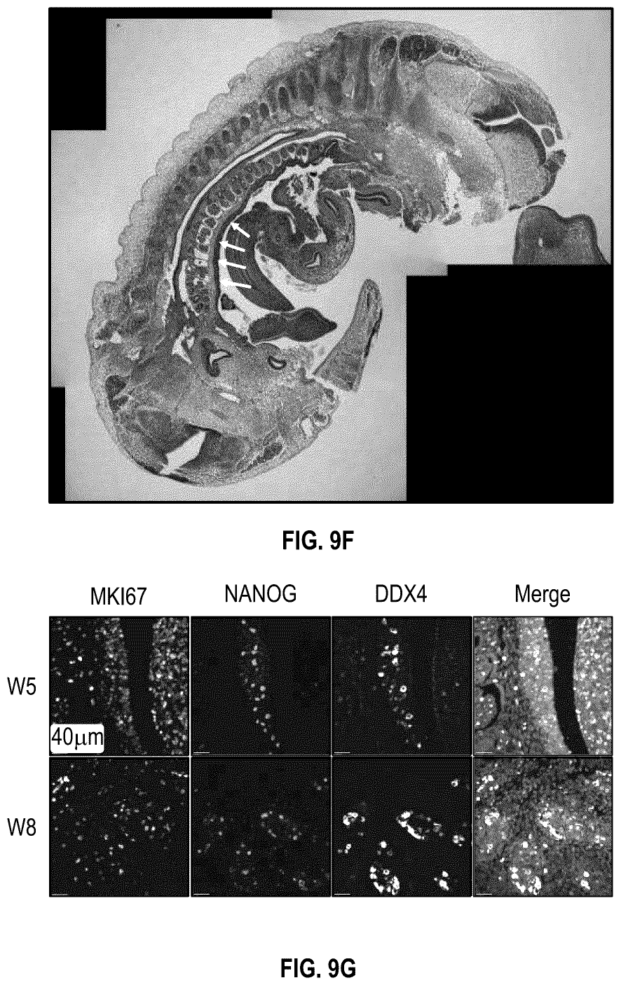

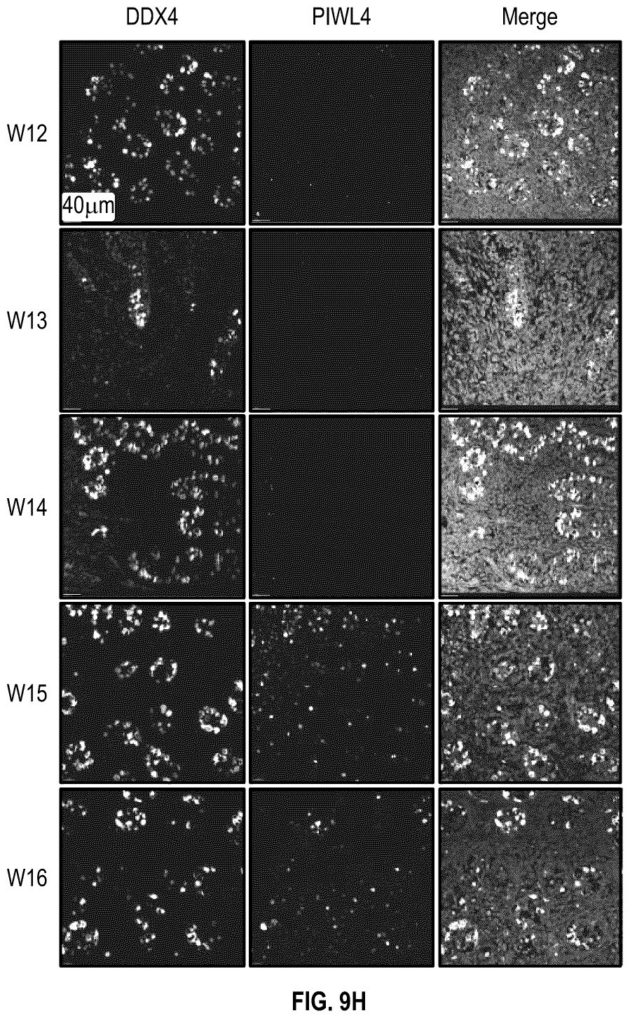

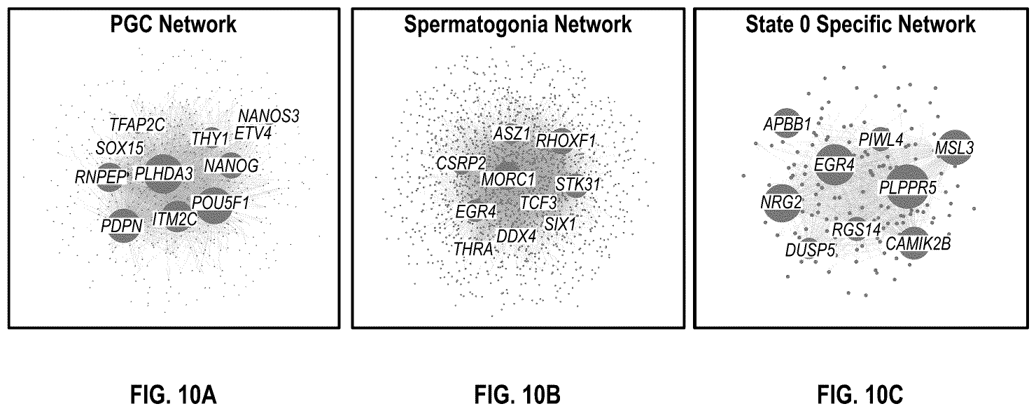

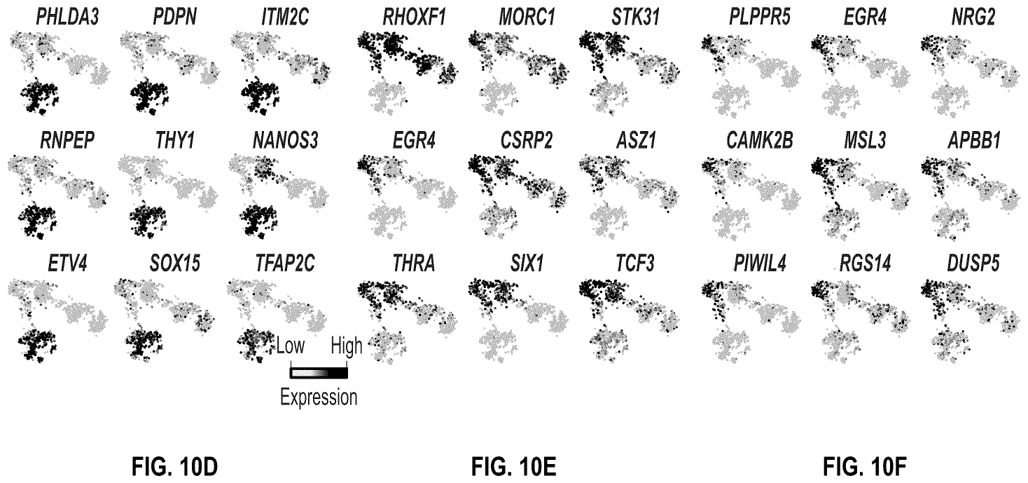

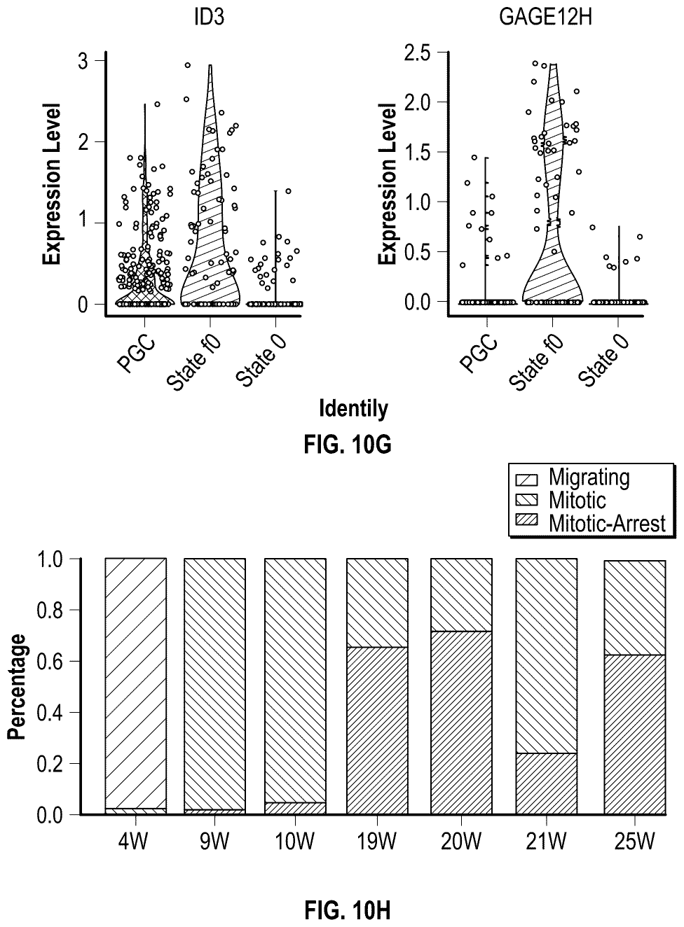

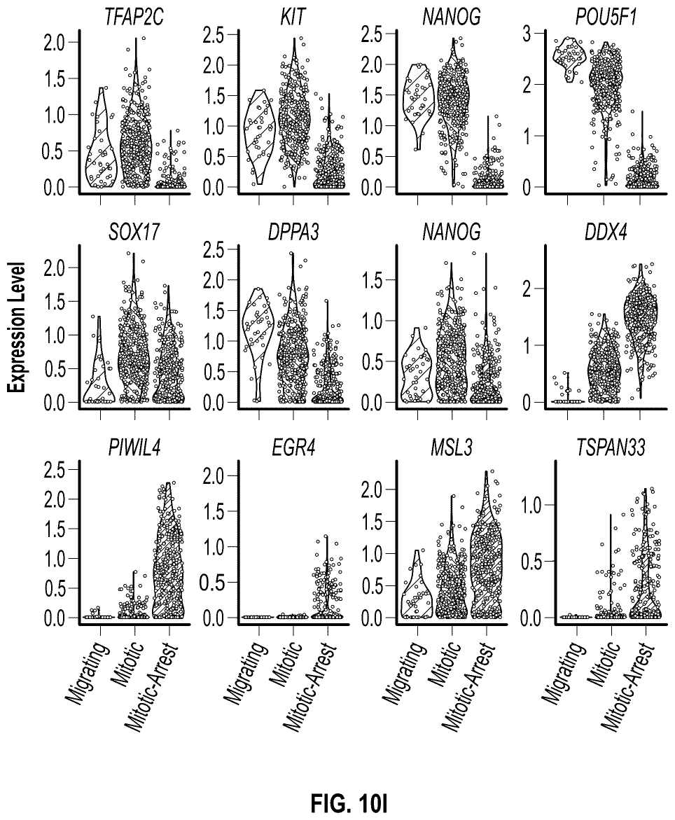

Development of the male germline was examined by parsing out and analyzing the germ cells separately from the somatic cells of the prenatal and postnatal (5 months) testes (cluster 12 from FIG. 1B). To place the embryonic, fetal, and postnatal germ cells in a more complete developmental timeline and enable comparisons, these data were combined with data from infant germ cells (1 year old) and adult spermatogonial states (states 0-4) from prior published work (Guo et al., 2018) by the inventors, which was also profiled on the 10× Genomics platform. A combination of dimension reduction (via t-distributed stochastic neighbor embedding [t-SNE]) and pseudotime analysis revealed seven defined clusters and a single pseudo-developmental trajectory that ordered and linked germ cells from the different stages (FIG. 2A). Following the order of pseudotime, it was observed that the first cluster of germ cells was largely composed of cells from 6 to 12 weeks, as well as a portion of germ cells from week 15 (FIG. 2A and FIG. 9A). This cluster was called the “embryonic-fetal group.” Their transcriptional identity is consistent with that of PGCs, including the expression of TFAP2C, KIT, NANOG, POUF51, SOX17, and others (FIG. 2B), which is consistent with prior scRNA-seq results (Li et al., 2017). The next developmental stage along pseudotime consists of cells from 15- and 16-week fetal samples that group together with cells from the 5-month- and 1-year-old postnatal samples, and was thus called the “fetal-infant group” (FIG. 2A and FIG. 9B). Interestingly, cells from the fetal-infant group lacked expression of the PGC markers mentioned above, and instead initiated the expression of multiple key state 0-specific markers (PIWIL4, EGR4, MSL3, TSPAN33, others), which were previously defined in the adult, infant, and neonatal testis. The subsequent clusters correspond to states 0-4 spermatogonia from adults, which display the sequential expression of markers associated with the subsequent developmental states: quiescent/undifferentiated (state 1; GFRA1⁺), proliferative (states 2-3; MKI67⁺, TOP2A⁺), and differentiating (state 4; SYCP3⁺) (FIG. 2A, 2B, and FIG. 9C), which is consistent with previous work by the inventors (Guo et al., 2017, 2018). This pseudotime order was further supported by orthogonal Monocle-based pseudotime analysis (FIG. 9D and FIG. 9E). A more systematic analysis via heatmap and clustering yielded 2,448 dynamic genes and provided a format to explore and display the identity, Gene Ontology (GO) terms, and magnitude of genes that show dynamic expression along this germ cell differentiation timeline (FIG. 2C). The embryo-fetal group (PGCs) displayed a high expression of genes (cluster 1) associated with signaling and gonad and stem cell development, which were then abruptly repressed between weeks 15 and 16, coinciding with the transition to the subsequent fetal-infant group. Here, the upregulation of many transcription- and homeobox-related genes (cluster 2) in the fetal-infant group, and the clear upregulation of markers of state 0 spermatogonia were also observe. Interestingly, the transition from the fetal-infant group to state 0 spermatogonia is characterized by a deepening and reinforcement of the state 0 gene expression signature, rather than a large number of new genes displaying upregulation. For example, differential gene expression analysis comparing fetal germ cells to adult state 0 spermatogonia identified only 2 genes (ID3 and GAGE12H; 2-fold, p<0.05) that display fetal-specific expression (FIG. 10G). Consistent with prenatal-postnatal similarity, germ cells from both younger and older infants located in the fetal-infant and adult state 0 clusters were observe. These results revealed that the spermatogonia present in young and older infants (called state 0) are highly similar to the fetal germline cells that emerge directly after PGCs exit the pluripotent-like state. Given this similarity, these were called fetal (f) cells state f0.

To validate the scRNA-seq profiles at the protein level, IF staining for key markers was performed. The proportion of NANOG⁺ (PGC marker) and MKI67⁺ (proliferation marker) decreased from 5 to 19 weeks (FIG. 2D and FIG. 9G), supporting the notion that the exit from the pluripotent-like state and entry into GO are temporally linked. It was noted that for NANOG, the loss of RNA signal based on transcription profiling appears more abrupt than the loss of protein, suggesting heterogeneity in the rates of protein loss. Regarding the acquisition of state 0 markers, no PIWIL4 positivity was detected in the 8- and 10-week samples; however, from week 14 onward, PIWIL4⁺ cells were clearly detected, specifically in DDX4⁺ germ cells (FIGS. 2E, 2F, and FIG. 9H). Thus, for the key pluripotency, proliferation, and state 0 markers tested, the IF staining results validate the scRNA-seq results.

### Network Expression Dynamics During Embryonic, Fetal, and Postnatal Germ Cell Development

To define candidate key genes and networks linked to germline developmental stages and transitions, network analysis was conducted. Using weighted correlation network analysis (WGCNA) (Langfelder and Horvath, 2008), gene-gene interactions that display dynamic expression patterns during PGC differentiation to state f0 spermatogonia were identified. Here, for the PGC up-regulated network (“PGC network;” FIGS. 10A and 10D), 2,126 genes and 122,360 interactions, and present the top 11 hub genes (and their interactions) were identified. As expected, several genes with known expression in PGCs were present, including POU5F1, NANOG, NANOS3, SOX15, and TFAP2C (Gkountela et al., 2015; Guo et al., 2015; Tang et al., 2015), confirming the robustness of the instant analysis. In addition, this analysis revealed PHLDA3, PDPN, ITM2C, RNPEP, THY1, and ETV4 as prominent markers in mitotic PGCs, providing candidates for future analysis. For example, PDPN, ITM2C, and THY1 encode cell surface proteins, and PDPN has successfully been used to isolate PGCs differentiated from human pluripotent stem cells (Sasaki et al., 2016). Regarding networks that accompany the differentiation of PGCs into state f0 spermatogonia, a large fraction of the identified genes show relatively broad expression within all subsequent spermatogonia stages, and thus this network was called the “spermatogonia network” (FIG. 10B and FIG. 10E). 771 genes and 31,557 interactions were identified, and the top 10 hub genes were presented. Here, roles for EGR4, DDX4, TCF3, and MORC1 in mammalian germ cells are well known. Interestingly, the analysis also indicates several additional factors (e.g., RHOXF1, STK31, CSRP2, ASZ1, SIX1, THRA) worthy of further exploration. For example, RHOXF1 mutations in humans confer male infertility (Borgmann et al., 2016), and MORC1 and ASZ1 both play important roles in protecting the germline genome by repressing transposable element activity (Ma et al., 2009; Pastor et al., 2014), raising the possibility that they may coordinate with the PIWIL4 factor described below. The networks that were exclusively expressed in state 0 SSCs (“state 0 network”; FIG. 10C and FIG. 10F) were also examined. 190 genes and 8,841 interactions were identified, and the top 9 hub genes were presented. Among them, EGR4, CAMK2B, MSL3, PLPPR5, APBB1, and P!W!L4 were already identified in prior work (Guo et al., 2018; Sohni et al., 2019), whereas here, NRG2, RGS14, and DUSP5 emerge as additional factors. Thus, the instant analysis confirms the roles of many known factors and provides a list of key candidate genes with less-studied functions in germ cell development, providing multiple avenues for future studies.

### Embryonic Specification and Fetal Development of Interstitial and Sertoli Lineages

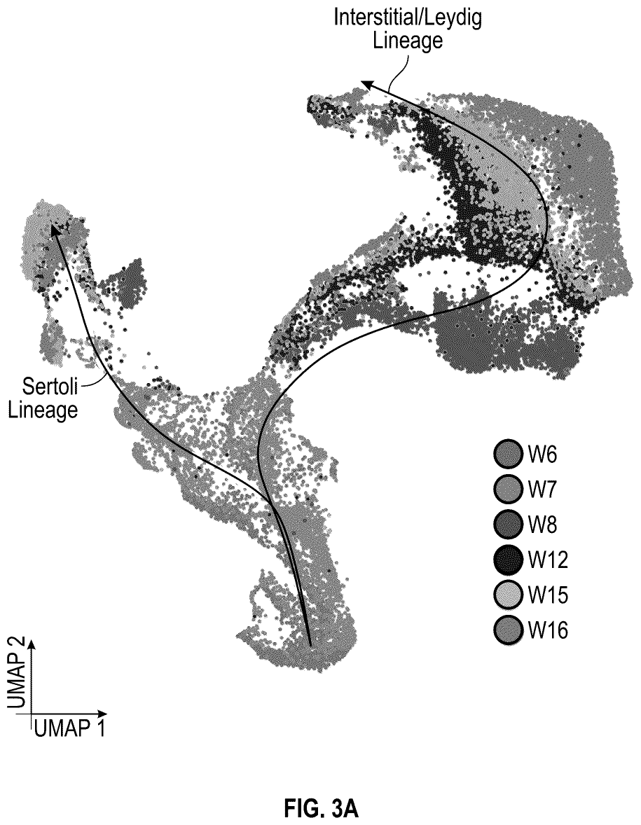

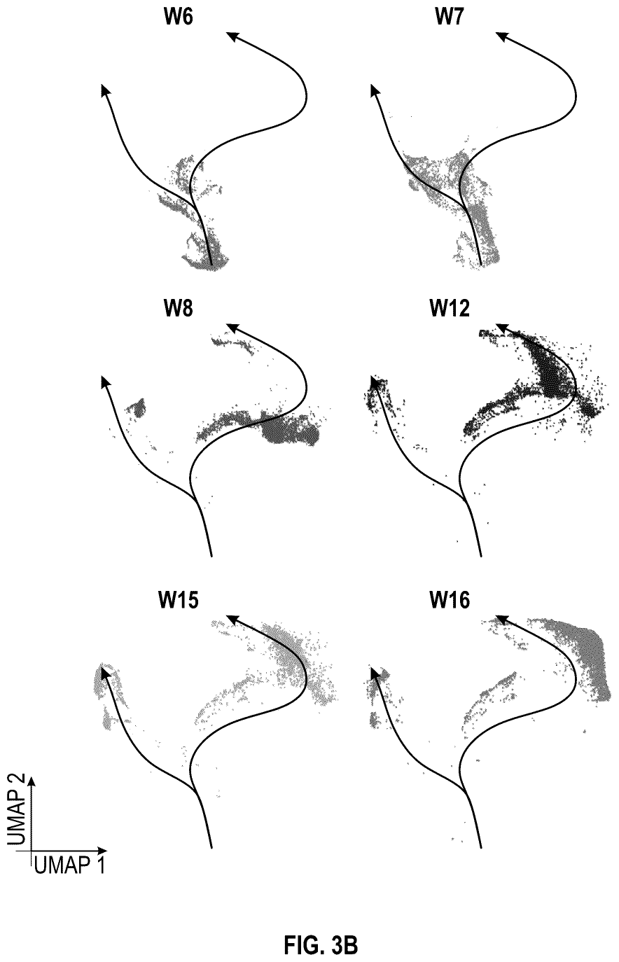

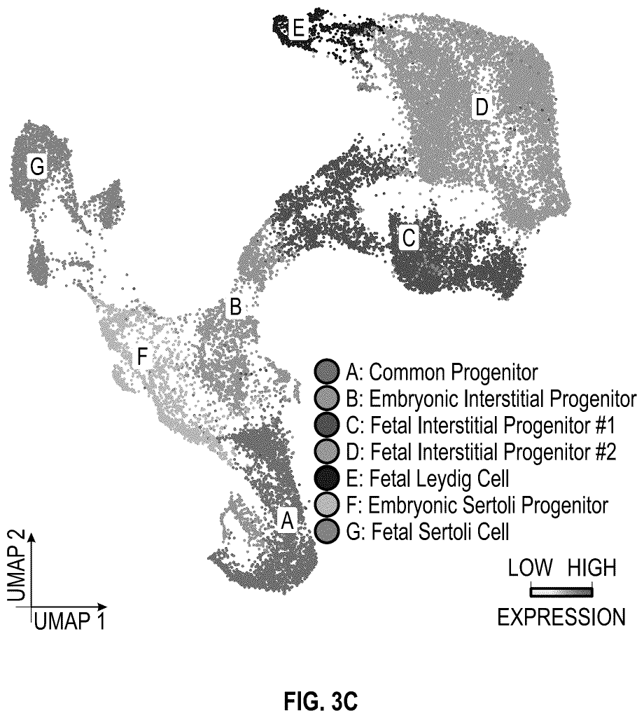

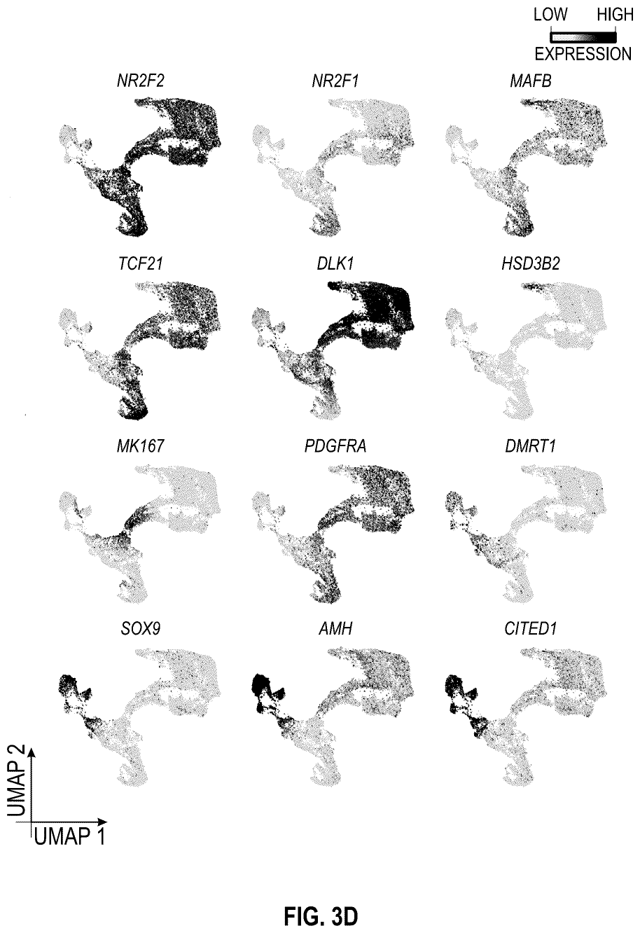

The cell type analyses revealed that the human embryonic and fetal testis stages consist primarily of somatic niche cells, including Sertoli cells and interstitial cells (including Leydig cells) (FIGS. 7B and 7C). Notably, cells that resemble fetal myoid cells by examining myoid markers, including ACTA2 and MYH11 were not observed, which contrasts with observations in mice (Wen et al., 2016). Here, the profiling of early embryonic (weeks 6-7) testes provided the opportunity to examine Sertoli and interstitial/Leydig cell specification. To this end, the fetal somatic niche cells that belong to the interstitial/Leydig and Sertoli lineages were parsed out, along with the early cells of indeterminate cell type (clusters 1-8 and 10 from FIG. 1B), and further analysis was performed. Interestingly, reclustering and subsequent pseudotime analysis revealed one cell cluster at early pseudotime, which transcriptionally bifurcates into two distinct lineages later in pseudotime (FIG. 3A). Notably, the early cluster was composed exclusively of cells from weeks 6-7, whereas cells from week 7 onward align along 2 distinct paths (FIGS. 3A, 3B, and 11A). Examination of known markers suggested that the 2 developmental paths represent Sertoli (left trajectory) or interstitial/Leydig (right trajectory) lineages, respectively (FIGS. 3C and 3D), and the existence of a heterogeneous pool of cells at weeks 6-7 from which both of these trajectories originate, raising the possibility of a common somatic progenitor population. Based on the clustering analysis, the embryonic-fetal interstitial and Sertoli development were then classified into seven stages (A-G), beginning with candidate common somatic progenitors (A) that differentiate into embryonic interstitial/Leydig progenitors (B), which undergo active proliferation (expressing high MK!67). The mostly quiescent embryonic Sertoli progenitors emerge at around week 7 (F). The embryonic interstitial progenitors (A) appear to differentiate into fetal interstitial progenitors (C and D) and also fetal Leydig cells (E), and embryonic Sertoli progenitors will differentiate into fetal Sertoli cells (G). Thus, the computational analysis suggests a heterogeneous multipotential progenitor for interstitial cells and Sertoli cells at 6-7 weeks, which then differentiates into Sertoli and interstitial (including Leydig) lineages between weeks 7 and 8.

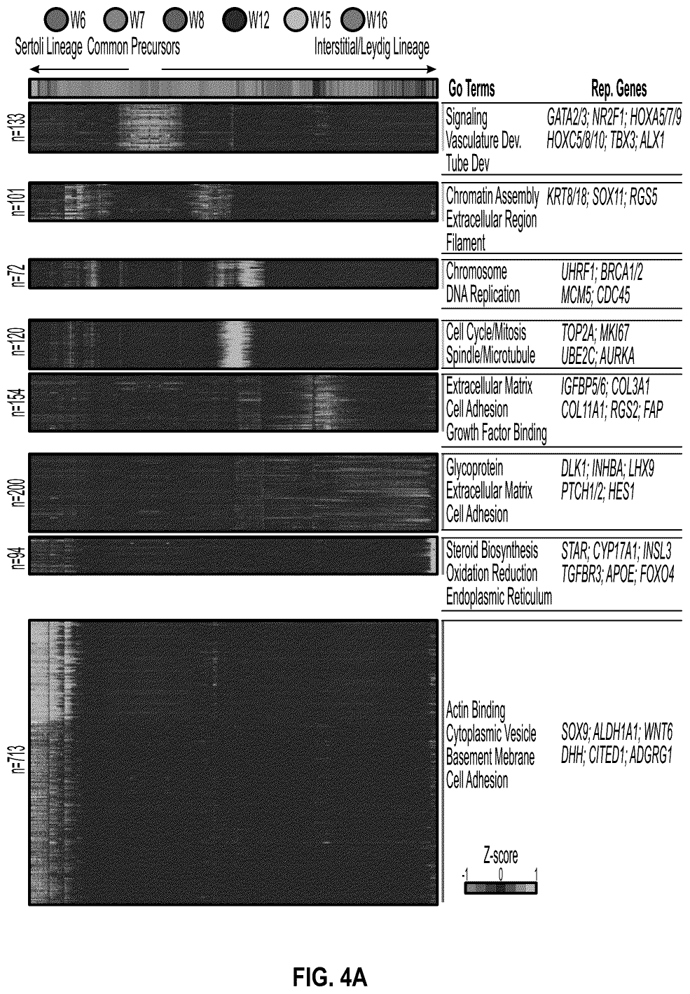

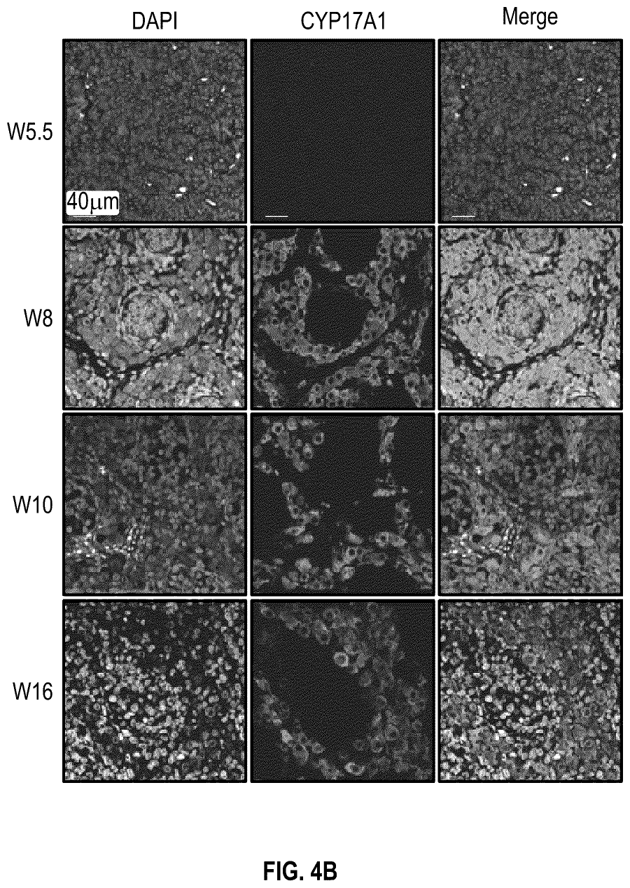

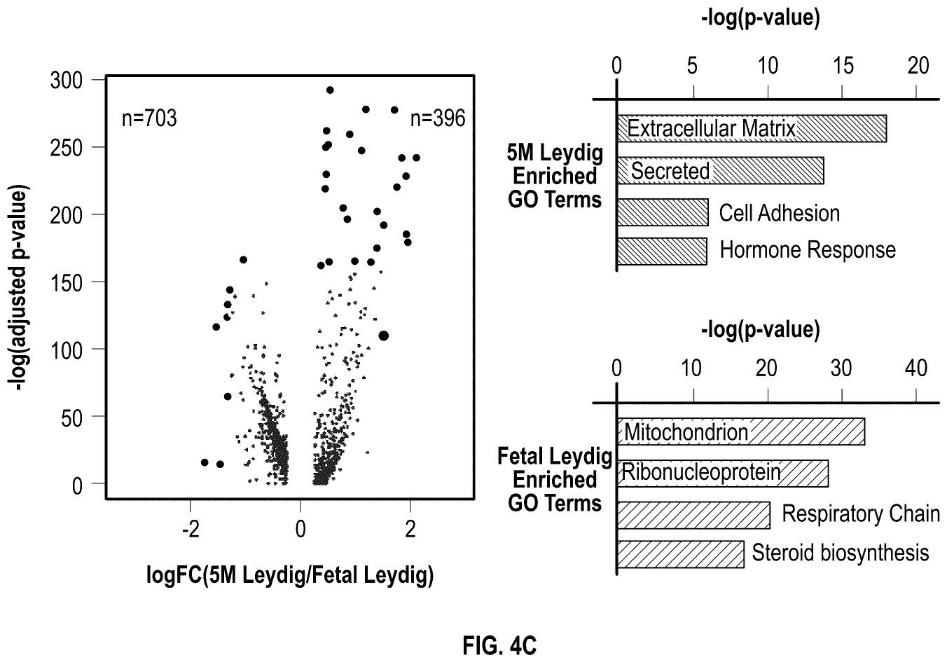

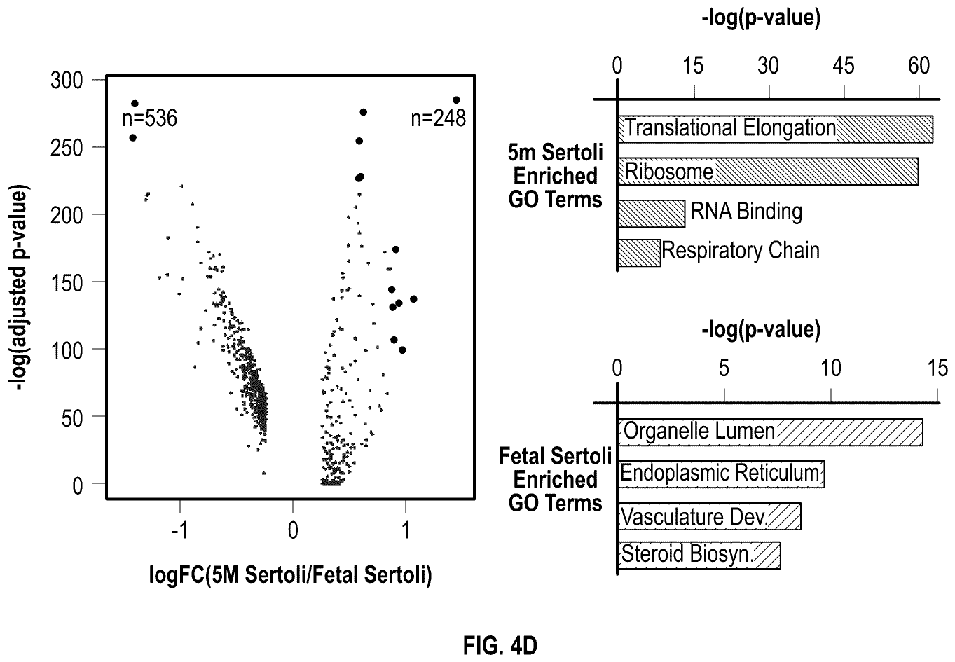

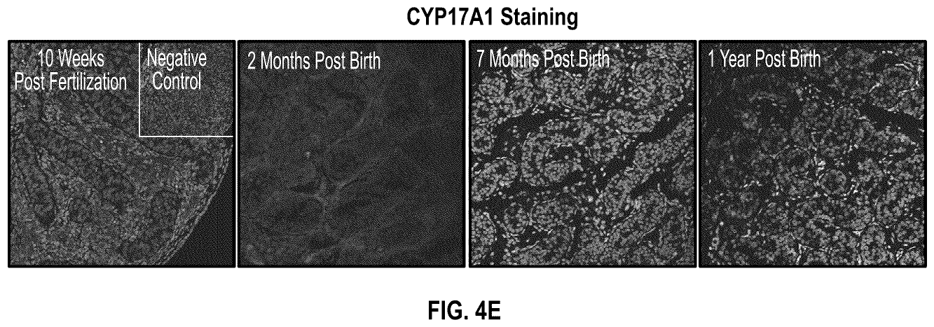

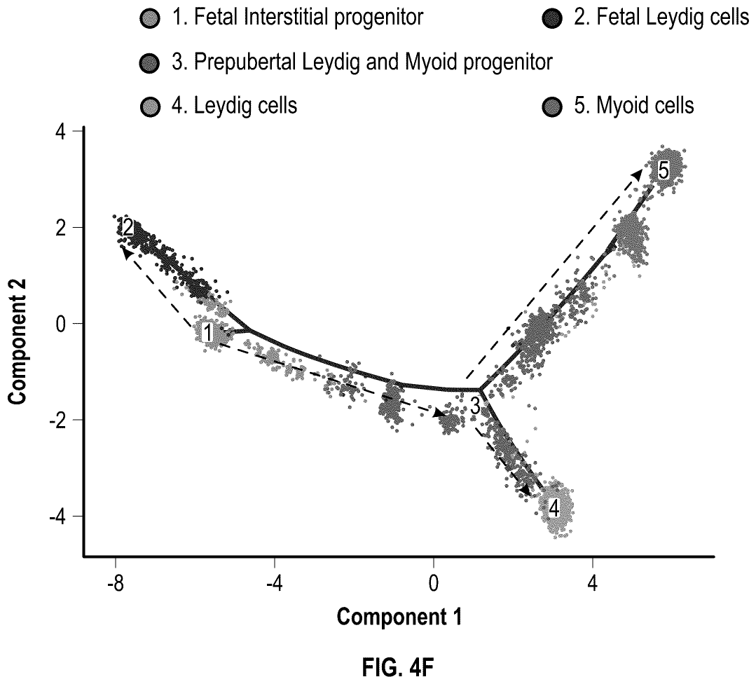

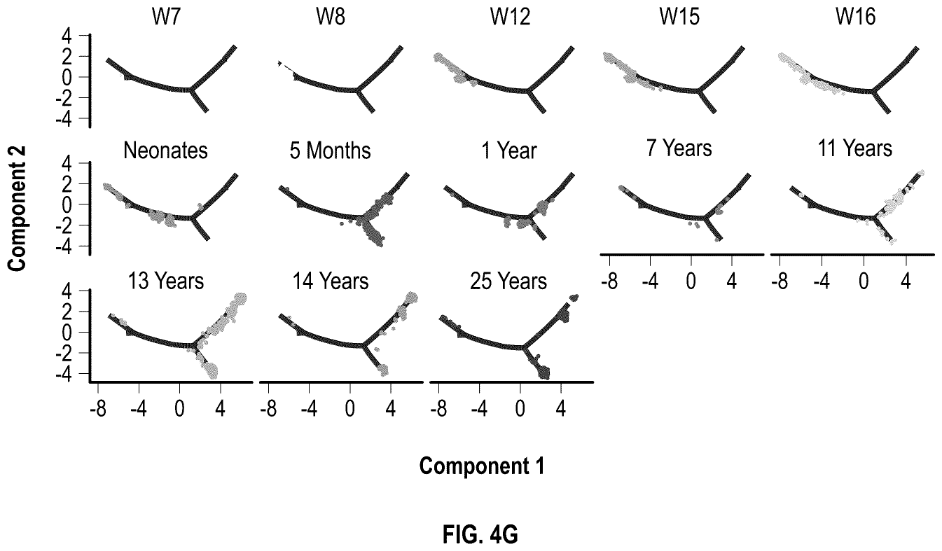

To further define the gene expression programs that accompany male sex determination, gene expression clustering analysis (k-means) was performed to identify the gene groups that display dynamic expression patterns along the pseudotime developmental trajectories (FIG. 4A). Notably, the candidate progenitors (at weeks 6-7) express multiple notable transcription factors, including GATA2, GATA3, NR2F1, HOXA, and HOXC factors and others, with enriched GO terms that include signaling and vasculature development. In particular, several genes involved in tube development (e.g., TBX3, ALX1, HOXA5) are specifically expressed in these candidate progenitors, which is consistent with the initiation of tubule formation to create the testis cords at week 6 (FIG. 4A and FIG. 11B).

This population of cells then bifurcates into distinct transcriptional programs consistent with embryonic Leydig or Sertoli cell progenitors. Along the Sertoli lineage, expressed genes are associated with chromatin assembly, extracellular region, and filament formation. Along the Leydig lineage, cells first express genes related to DNA replication, proliferation, and cell cycle, indicating a phase of Leydig lineage amplification, consistent with a much higher number of cells present on the Leydig lineage trajectory at and after 8 weeks compared to the Sertoli lineage (FIGS. 3B, 4A, and 11A). This is followed in the Leydig lineage by the up-regulation of terms linked to extracellular matrix, cell adhesion and glycoproteins, and components and gene targets associated with both Notch and Hedgehog signaling. Consistent with the known roles of fetal Leydig cells in androgen production in mice (Shima et al., 2013, 2015), fetal Leydig cells placed at the end of pseudo-time express high levels of genes related to steroid biosynthesis (e.g., HSD3B2: FIG. 3D) and secretion. Interestingly, these cells emerge very early, by week 7, and persist for the remainder of the stages examined, suggesting both an early and a persistent role. For the Sertoli lineage, the fetal Sertoli cells express high levels of genes associated with structural functions. To validate the temporal features of steroidogenesis genes, IF staining of CYP17A1, a marker for steroidogenesis highly expressed in fetal Leydig cells was performed (Shima et al., 2013; FIGS. 4B and 11D). Notably, it was found that CYP17A1 is absent in the genital ridge epithelium at 5.5 weeks, whereas robust staining is observed in the interstitial (non-cord) areas in all samples at R7 weeks, strongly suggesting that Leydig cell specification occurs at around week 7, consistent with the scRNA-seq findings herein. Furthermore, it was observed that at week 8, not all interstitial cells are positive for CYP17A1. Here, it was speculated that the fetal CYP17A1⁻ interstitial cells may be the interstitial cell population that gives rise to postnatal Leydig and peritubular cells.

### Relationship Between Fetal and Infant Leydig and Sertoli Cells

The datasets provided an opportunity to compare and contrast fetal versus postnatal human Leydig and Sertoli cells. 396 or 703 genes were found to be differentially expressed (upregulated or down-regulated, respectively) when comparing fetal to infant Leydig cells, respectively (bimodal test; adjusted p<0.01; |log FC|>0.25) (FIG. 4C). As Leydig cells transition from fetal to infant, genes associated with the extracellular matrix, secretion, cell adhesion and hormonal response are upregulated, while those with mitochondrial function and steroid biosynthesis (e.g., CYP17A1, HSD3B2, STAR) are downregulated (FIG. 4C). Likewise, 536 or 248 genes differentially expressed in the infant or fetal Sertoli cells, respectively were found (FIG. 4D). As Sertoli cells transition from fetal to infant, genes associated with translation and respiratory chain are upregulated, and these cells with endoplasmic reticulum and steroid biosynthesis are downregu-lated (FIG. 4D). To confirm, IF staining of CYP17A1 was performed (Shima et al., 2013) and its expression was found to be undetected in the postnatal samples (FIG. 4E), suggesting that fetal Leydig cells disappear or differentiate after birth in humans, which is consistent with discoveries in mice (Svingen and Koopman, 2013). The results suggest that human fetal Leydig and Sertoli cells both exhibit expression of steroid biosynthetic genes, whereas this property is downregulated in the postnatal samples tested.

Prior work by the inventors based on juvenile human testes showed that Leydig and myoid cells share a common progenitor at prepubertal stages (Guo et al., 2020). To gain a deeper understanding of the relationship between the fetal interstitial progenitors and prepubertal Leydig/myoid progenitors, as well as insight into how the common progenitor for the Leydig and myoid lineage is specified from fetal and postnatal precursor cells, additional analysis was performed. Here, in silico scRNA-seq datasets from fetal interstitial cells (clusters C, D, and E from FIG. 3C), neonatal Leydig cells (Sohni et al., 2019), and the postnatal and adult Leydig/myoid cells (Guo et al., 2020) were combined. Following cell combination, Monocle pseudotime analysis, which aims to provide the developmental order of the analyzed cells through computational prediction was performed (FIGS. 4F and 4G). Here, the pseudotime trajectories (depicted by the dashed arrows in FIG. 4F) agree nicely with developmental order based on age (FIG. 4G), suggesting that fetal interstitial progenitor cells give rise to the postnatal and prepubertal Leydig/myoid progenitor cells. In addition, the analysis suggests that the fetal Leydig cells, which originate from the fetal interstitial progenitors, are absent in the postnatal and infant stages, a result confirmed by the immunostaining data (FIG. 4E).

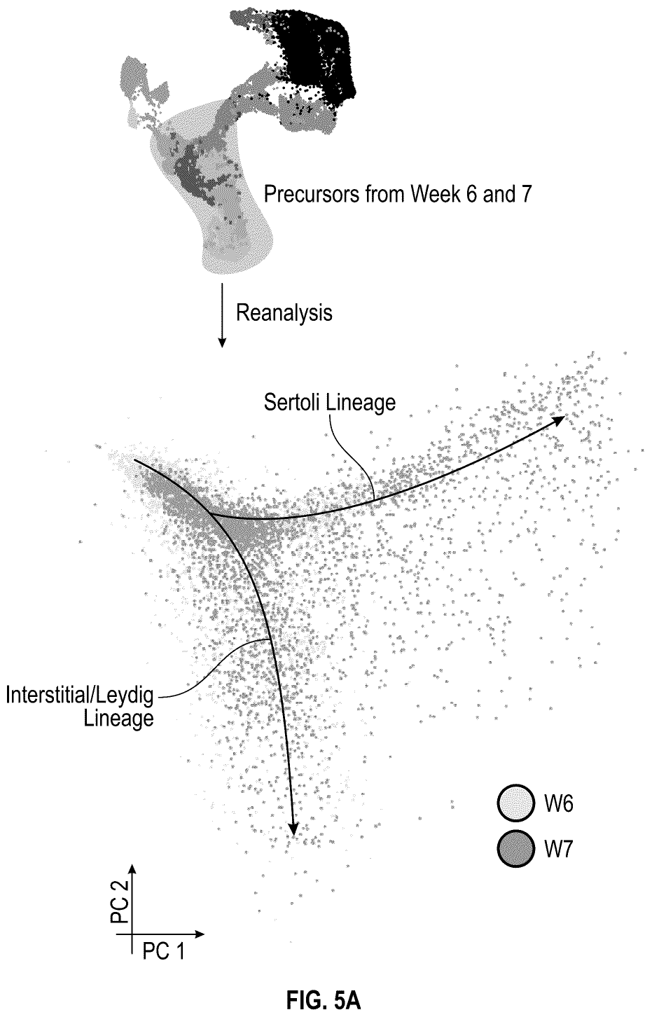

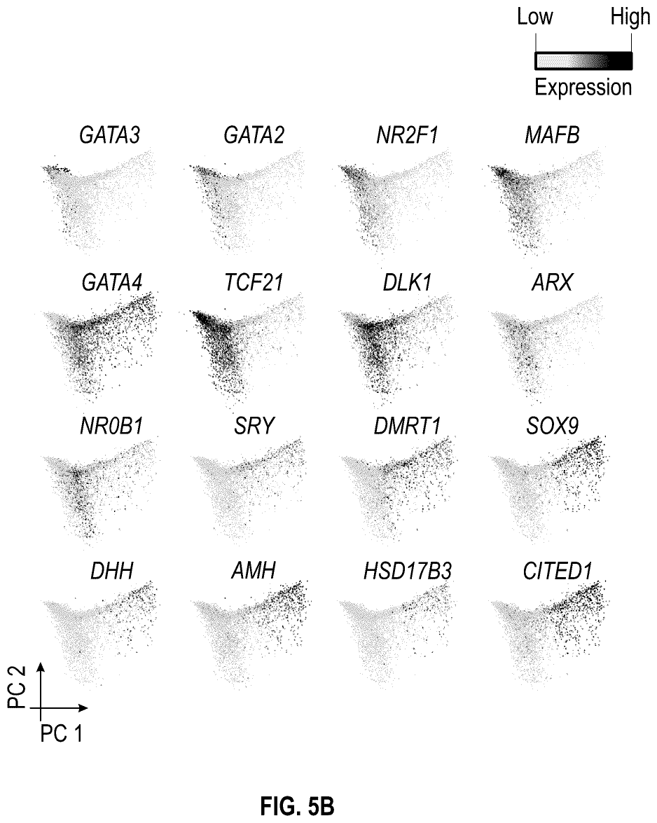

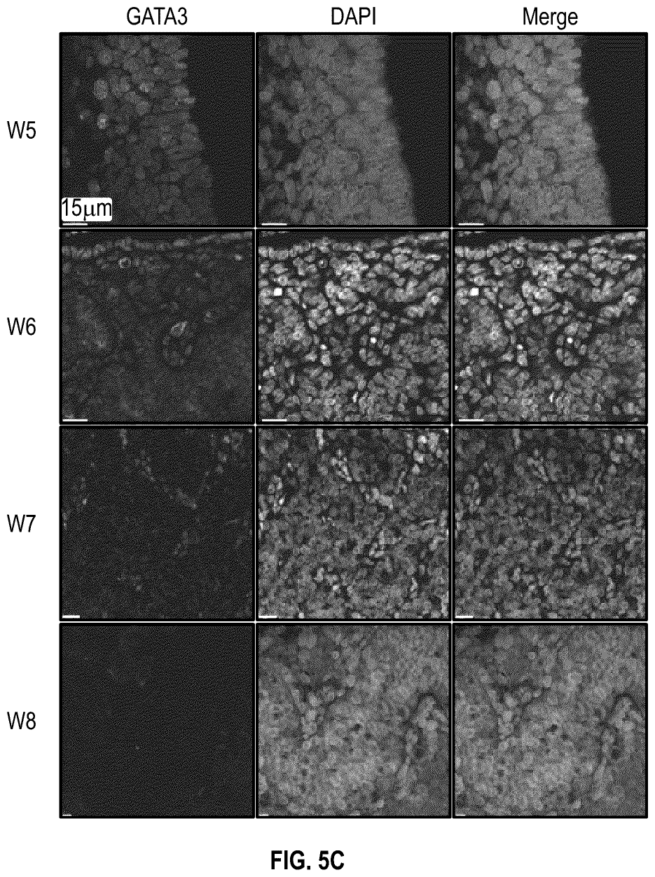

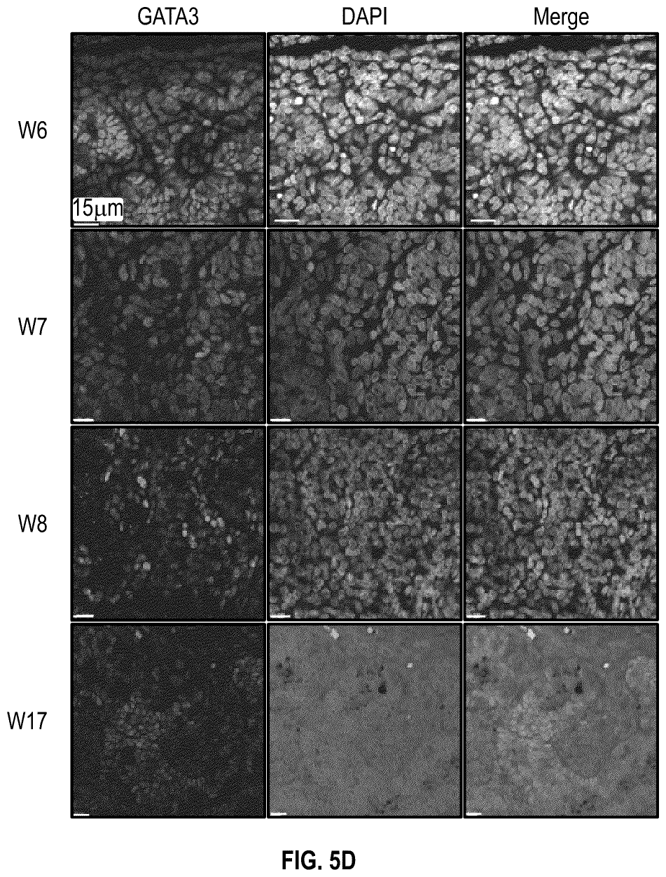

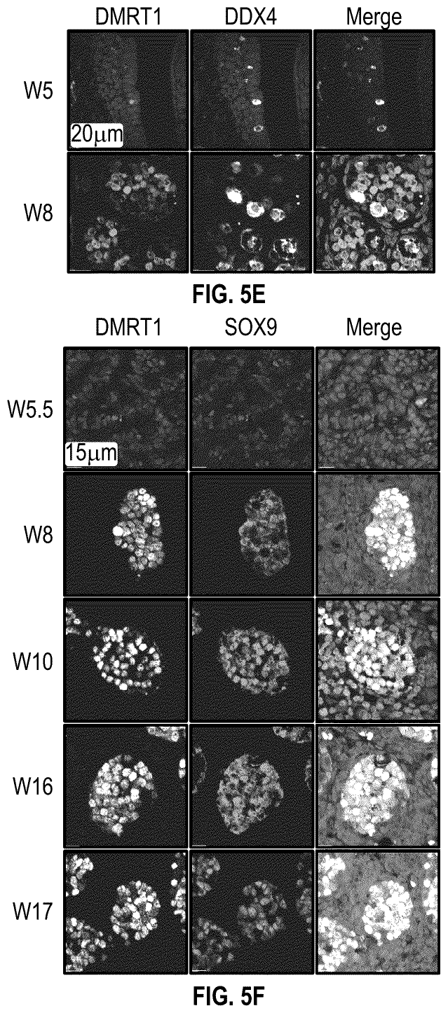

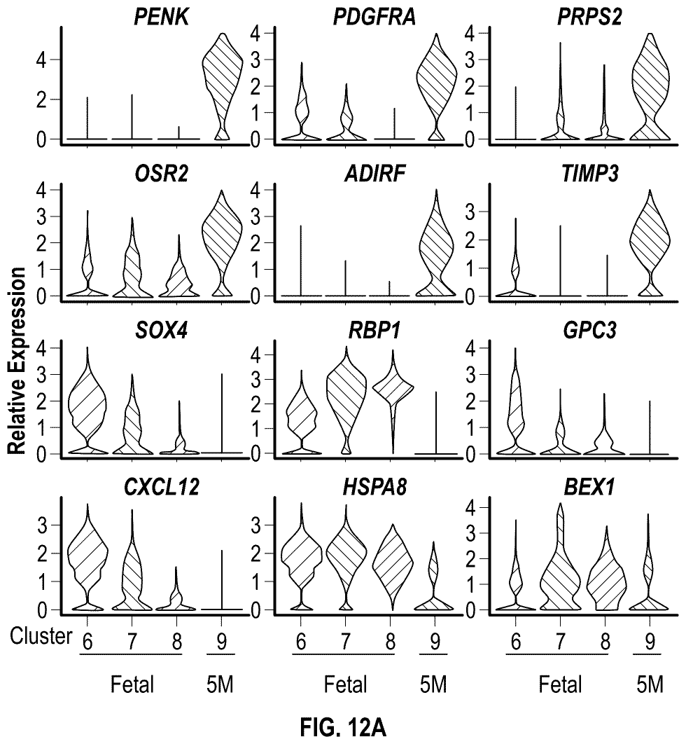

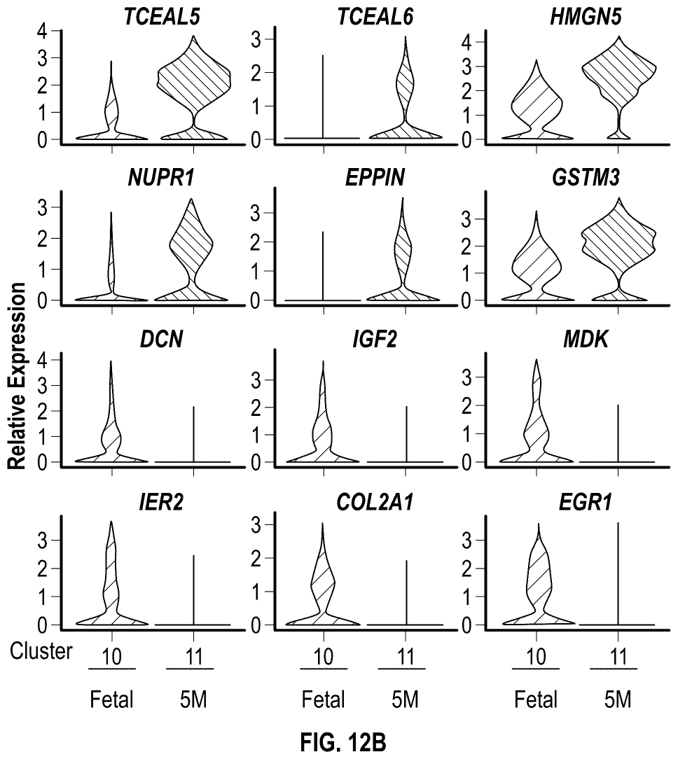

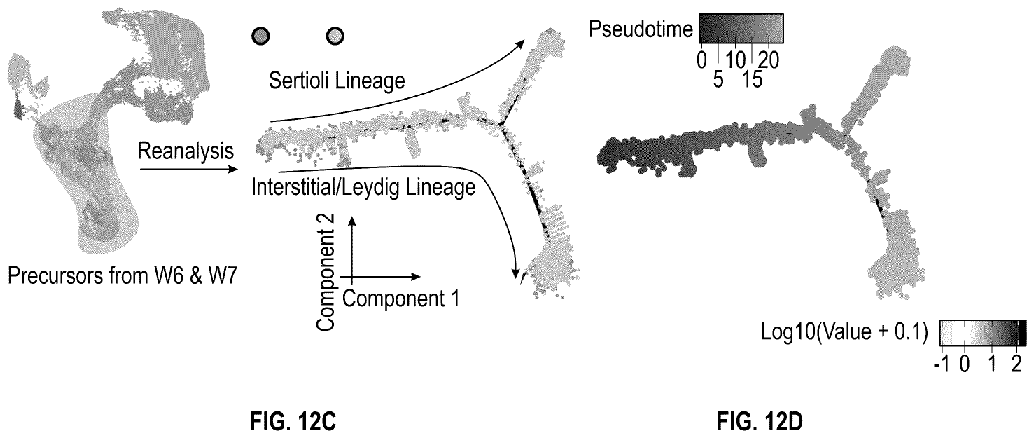

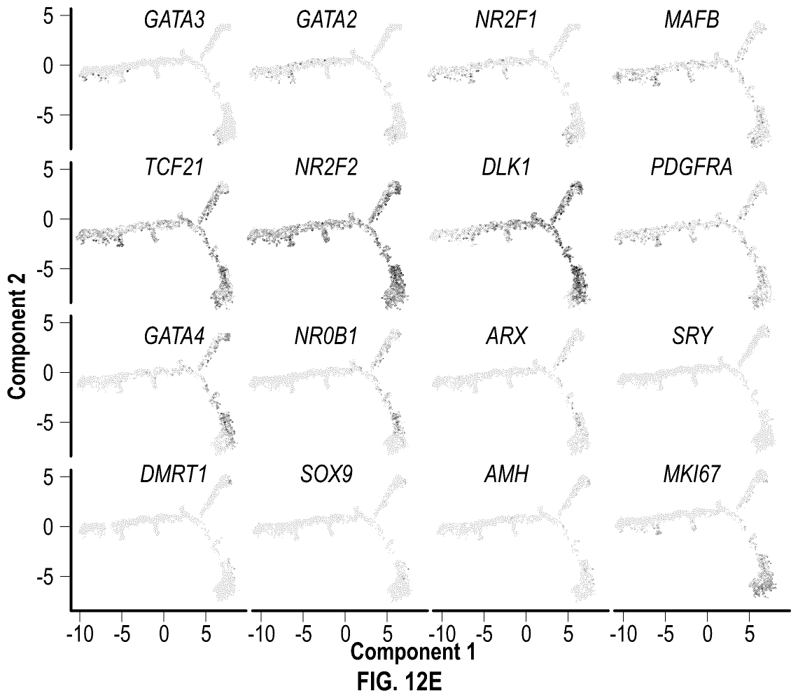

Key Factors Correlated with Embryonic Specification of Interstitial and Sertoli Lineages

Whereas testicular niche cells from 8 to 16 weeks expressed transcription factors characteristic of advanced interstitial or Sertoli cell lineages, the cells from the 6-week gonads lack these late markers, which initially emerge at week 7 (FIGS. 3A-3C). To better understand the genes expressed during the time of somatic specification, the 6- and 7-week cells were parsed out (from FIG. 3A) and a more detailed analysis was performed. Here, principal-component analysis (PCA) of the 6- and 7-week cells revealed that a large portion of the cells did not display markers distinctive for either interstitial or Sertoli cells (FIG. 5A), suggesting a heterogeneous population in which the Sertoli and Ley-dig/interstitial precursors are emerging. An orthogonal analysis via Monocle also confirmed similar patterns and properties (FIG. 12C-12E). Based on the gene expression patterns (FIG. 5B), it was possible to assign the cells at the bottom as the embryonic interstitial/Leydig lineage (expressing DLK1 and TCF21), and the cells at the top right as the embryonic Sertoli lineage (expressing SRY, DMRT1, SOX9, AMH, and others).

Next, it was sought to identify candidate key transcription factors that may participate in initial somatic lineage specification (FIG. 5B). Interestingly, a set of GATA family factors displayed sequential and largely non-overlapping patterns: GATA3 expression was earliest, at the top and left edge of the PCA plot (mostly 7 week), GATA2 started to express somewhat later, and GATA4 was expressed in a later population that was progressing toward the Sertoli lineage. Many other factors also display sequential expression. For example, NR2F1, MAFB, and TCF21 show relatively early expression (similar to GATA2), while TCF21 expression persists through the development of the Leydig lineage, but not the Sertoli lineage. Notably, both ARX and NR0B1 are expressed at the bifurcation stage. For the Sertoli lineage, these early markers cease expression at lineage specification, followed by the expression of SRY and DMRT1 as the earliest markers of the lineage, and then followed by SOX9.

Finally, extensive IF was performed to validate the genomics findings. GATA3 was observed throughout the genital ridge epithelium at week 5, which became restricted to a subpopulation of interstitial cells at weeks 6-7, and by week 8, GATA3 protein becomes undetectable (FIG. 5C). In addition, GATA4 expression is evident both inside and outside the cords from week 6 and onward (FIG. 5D and FIG. 11B). To evaluate Sertoli lineage specification, staining was performed for DMRT1 alongside either a germ cell marker (DDX4) or an additional Sertoli cell marker (SOX9) (FIGS. 5E and 5F). As expected, DMRT1 and SOX9 protein were undetectable in the GATA3/GATA2⁺ genital ridge epithelium containing DDX4⁺ PGCs at week 5 (FIG. 5E). However, by 8 weeks (after cord formation), DMRT1⁺ and SOX9⁺ Sertoli cells are identified (FIG. 5F). Taken together, the IF staining results confirm key markers discovered through the genomics approaches and provide additional insights into the physiology of testis cord development in the embryonic and fetal stages.

### Discussion

PGCs are specified in the early embryo, followed by migration to the genital ridge (Chen et al., 2019; Tang et al., 2016; Witchi, 1948). The genital ridge then undergoes exquisite developmental programming to form the somatic cells of the testicular niche that support the survival and differentiation of the male germline during fetal life. Although prior studies from mice provide rich knowledge of the formation and lineage specification in the embryonic testis (reviewed in Svingen and Koopman, 2013), understanding of human embryonic and fetal testis development has been much less studied, particularly in regard to the specification of the somatic lineages. Here, through the application of single-cell sequencing of unselected testicular cells, together with IF staining, a detailed molecular overview of human fetal testis development is provided, to help delineate the temporal molecular changes involved in human embryonic and fetal testis development and further differentiation.

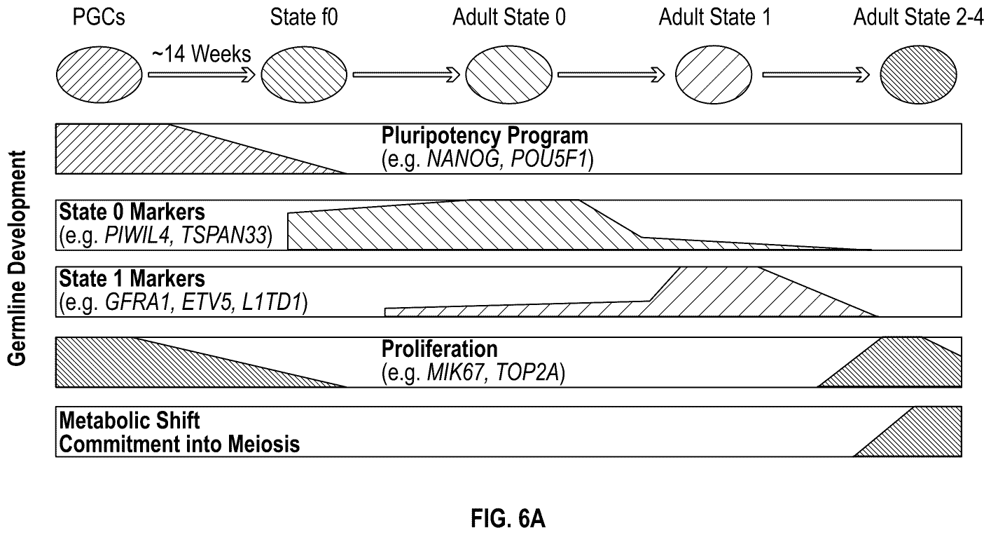

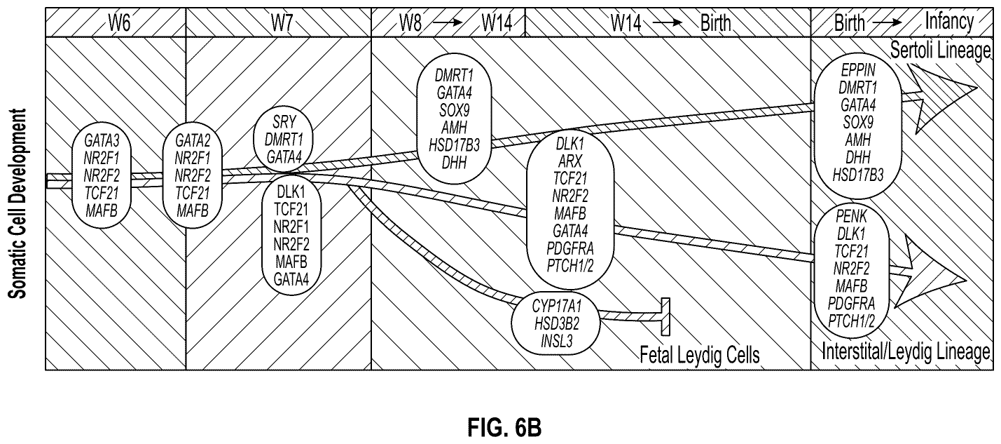

One critical question it was aimed to address is the transition of PGCs into spermatogonia, specifically the transcriptional relationship of differentiating male human PGCs during fetal life to postnatal state 0 SSCs, which have been identified as the most undifferentiated male germline stem cells in human infants and adults (Guo et al., 2018; Sohni et al., 2019), as well as primates (Shami et al., 2020). Combined with prior work (Guo et al., 2017, 2018, 2020; Sohni et al., 2019), the current work provides an evidence-based and detailed model for human germline development that spans embryonic, fetal, infant, pubertal, and adult stages (FIG. 6A). During 6-12 weeks postfertilization, as the male somatic cell linages are being specified, human male PGCs express high levels of transcription factors associated with pluripotency (e.g., POU5F1, NANOG), together with classic well-characterized PGC transcription factors (e.g., SOX17, TFAP2C) and are proliferative. At 14 weeks, a subpopulation of PGCs initiates repression of the pluripotency-like program, and extinguishes expression of the early PGC genes (Li et al., 2017), while simultaneously turning on the state f0 spermatogonia programs (e.g., P!W!L4, MSL3, EGR4, TSPAN33). These state f0 spermatogonia are transcriptionally highly similar to the state 0 spermatogonia, and are found from fetal stages through infants within the seminiferous cords. Interestingly, when the expression patterns of many key PGC or state f0 markers in a prior FGC dataset were examine (Li et al., 2017; FIG. 10H), it was found that the mitotically arrested FGCs exhibit specific and high expression of state 0 genes (e.g., PIWIL4, EGR4, MSL3, TSPAN33) and low expression of PGC genes (e.g., POU5F1, NANOG, TFAP2C, SOX17). This observation strongly suggests that the previously defined mitotically arrested FGCs (Li et al., 2017), which also emerge at 14 weeks postfertilization (FIG. 10I), are likely the same cells as the state f0 defined in the study. Here, the prior derivation of infant state 0 cellular identity and their demonstrated similarity to the fetal population in the present study defines a critical linkage: PGCs differentiate and transition into state f0 spermatogonia and reinforce their state 0-like transcriptome as they transition between fetal germ cells and postnatal germ cells. By 5 months, all of the germline cells display a state 0 spermatogonial transcriptome, and cells with a PGC transcriptome are below the limit of detection. Consistent with the observations at 5 months and in infants, state 0 markers are also expressed in human neonatal germ cells (Sohni et al., 2019). It is revealed that state 0-like spermatogonia originate from PGCs at around weeks 14-16 of fetal life and persist through all of the prenatal and postnatal developmental stages, to provide a pool of undifferentiated spermatogonia in adults available for niche-guided transitions to more differentiated spermatogonial states and ultimately gametogenesis (FIG. 6A).

Prior work in mouse models has revealed several factors and pathways that play important roles in lineage specification and progression of testicular somatic cells in mice (Liu et al., 2016; Svingen and Koopman, 2013; Yao et al., 2002). Recently, scRNA-seq has proven to be a powerful tool to study embryonic and neonatal mouse testis development (Stévant et al., 2019; Tan et al., 2020). Here, the work demonstrates that several key factors in early somatic lineages (e.g., WT1, NR2F1, SOX9, SRY, DMRT1) are shared between humans and mice. Furthermore, through the systematic examination of prenatal human testes via single-cell profiling and IF staining, many additional candidate factors are provide for future characterization, and reveal multiple human-mouse differences. For example, through IF staining of the genital ridge epithelium, no evidence of Sertoli cell or Leydig cell identity was found before 6 weeks postfertilization. Then, starting at week 6, the unbiased/unselected single cell transcriptome profiling identified rare fetal Leydig- and Sertoli-like cells. A large, closely related population of cells that is heterogeneously positive for developmental transcription factors, notably NR2F1, GATA3, and GATA4 RNA was also identified in pseudotime a. GATA3 protein analysis demonstrated that GATA3 is uniformly expressed by the genital ridge epithelium at week 5 postfertilization before specification of Sertoli and Ley-dig cells. Notably, at week 6, when cord formation initiates, GATA3 expression is restricted to a subpopulation of cells in the interstitium. In counterdistinction, GATA4 expression is evident and broad at 6-7 weeks postfertilization, and remains detectable at 17 weeks postfertilization. In the mouse embryo, GATA4 is known to be critical for genital ridge formation, and in the absence of GATA4, the bipotential gonads do not form (Hu et al., 2013). Given that GATA3 is expressed in the genital ridge epithelium before GATA4, it is speculated that GATA3 may have a role in specifying the genital ridge in humans, whereas GATA4 instead may be involved in maintaining the somatic cell lineages after 6 weeks postfertilization, when GATA3 expression is reduced. In the mouse, NR5A1 (also called SF1) is another major transcription factor required for specifying the genital ridge epithelium (Hatano et al., 1996; Luo et al., 1994). However, clear expression of NR5A1 in the GATA3⁺ human progenitors was not observed, providing a second example in which formation of the genital ridge epithelium in human embryos appears different from the mouse (FIG. 11B). Analysis at the week 6-7 time point suggests that Leydig and Sertoli cell specification occurs at or near the same developmental time. The IF studies at week 7 show both Sertoli cells in cords and Leydig cells outside the cords. This result represents a major difference from the mouse, in which Sertoli cells are specified first, and then Leydig cells are subsequently specified (Svingen and Koopman, 2013). Considering that the size of the fetal human testis is proportionally much larger than that of mice, the human testis progenitors may commit relatively early in development, followed by waves of proliferation, which may partly explain the developmental differences.

In addition to being specified at an equivalent developmental stage, it was also discovered that the 6- and 7-week somatic niche progenitors expressed markers consistent with their ability to differentiate into interstitial/Leydig and Sertoli lineages by transiently expressing (in a small subset of cells) key transcription factors, including ARX, NR0B1, or SRY. This identity is further reinforced at 8 weeks, when all cells are distinguishable as interstitial/Leydig or Sertoli lineage cells. Notably, the establishment of the male somatic cell lineages in the embryonic testis occurs almost 2 months before the PGCs begin differentiating into state f0 (at 14-18 weeks). In contrast, in mice, there is only a 2-day delay in the timing of the male niche cell differentiation (at day 12) to the initiation of mouse PGC differentiation into prospermatogonia (at embryonic day 14) (Saitou and Yamaji, 2012; Svingen and Koopman, 2013; Western et al., 2008). The purpose of this 2-month delay in which human PGCs are shielded from initiating differentiation into state f0 spermatogonia in the seminiferous cord niche may be related to the need to increase the number of male germ cells through proliferation, given that these cells are MKI67⁺, before initiation of state f0 differentiation and male-specific epigenetic reprogramming (FIG. 6B).

The testis produces gametes in adult males through continuous niche-guided differentiation of SSCs, and a deep understanding of this biology is needed to improve male reproductive health. Here, the work provides major insights into defining the timing and strategy of human testis formation and its development before and after birth. Notably, the state f0 germ cells that emerge at 15 weeks during fetal life display remarkable similarities to the infant and adult state 0 cells, and thus allow us to link and depict the complete developmental progression of PGCs to adult state 0 cells. Furthermore, detailed molecular characterization of a common somatic progenitor pool and its amplification and transition to testicular niche cells, as well as initial insights into testicular cord formation and possible roles in guiding germ cell development are provided. These results should provide a foundation for future hypothesis-driven research, and could also help guide the reconstruction and study of the human early testis in vitro.

### Methods

Key resource table

| REAGENT or RESOURCE | SOURCE | IDENTIFIER |
|---|---|---|
| Antibodies | | |
| Rabbit polyclonal anti-PIWIL4, | Thermo Fisher | Cat#: PA5-3144, RRID: |
| Dilution: 1:200 | Scientific | AB_2548922 |
| Mouse monoclonal (CloneB56) | BD Biosciences | Cat#: 556003, RRID: |
| anti-MKI67, Dilution: 1:200 | | AB_396287, |
| Goat polyclonal anti-DDX4, | R&D Systems, | Cat#: AF2030, RRID: |
| Dilution: 1:100 | | AB_2277369 |
| Rabbit monoclonal (D73G4) anti- | Cell Signaling | Cat#: 4903, RRID: |
| NANOG, Dilution: 1:100 | Technology | AB_10559205, |
| Mouse monoclonal anti-CYP17A1, | Santa Cruz | Cat#: SC-374244, RRID: |
| Dilution: 1:200 | Biotechnology, | AB_10988393 |
| Mouse monoclonal (1A12-1D9) | Thermo Fisher | Cat#: MA1028, RRID: |
| anti-GATA3, Dilution: 1:100 | Scientific | AB_2536713, |
| Mouse monoclonal (G-4) anti- | Santa Cruz | Cat#: SC-25310, RRID: |
| GATA4, Dilution: 1:100 | Biotechnology, | AB_627667 |
| Mouse monoclonal anti-DMRT1, | Santa Cruz | Cat#: SC-377167 |
| Dilution: 1:100 | Biotechnology, | |
| Rabbit polyclonal anti-SOX9, | Millipore, | Cat#: AB5535, RRID: |
| Dilution: 1:200 | | AB_2239761 |
| AF488 goat-anti mouse IgG2a | Invitrogen | Cat#: A21131, RRID: |
| | | AB_2535771 |
| AF594 donkey-anti-mouse IgG | Invitrogen | Cat#: A21203, RRID: |
| | | AB_2535789 |
| AF594 goat-anti-mouse IgG1, | Invitrogen | Cat#: A21125, RRID: |
| | | AB_2535767 |
| AF594 donkey-anti-rabbit IgG, | Jackson | Cat#: 711-585-152, RRID: |
| | ImmunoResearch | AB_2340621 |
| AF647 donkey-anti-goat IgG, | Invitrogen | Cat#: A21447, RRID: |
| | | AB_2535864 |
| Biological samples | | |
| Human testis samples from | DonorConnect | N/A |
| postnatal donors | | |
| Human testis samples from | University of | N/A |
| embryonic and fetal stages | Washington- Birth | |
| | Defects Research Lab | |
| Human testis samples from Jan's | Karolinska Institutet | N/A |
| lab | | |
| Deposited data | | |
| Single cell RNA-seq for embryonic | This paper | GEO: GSE143356 |
| and fetal human testes | | |
| Single cell RNA-seq for postnatal | This paper | GEO: GSE161617 |
| testes | | |
| Software and algorithms | | |
| Seurat (2.3.4) | Butler et al., 2018 | https://satijalab.org/seurat/ |
| Monocle (2.10.1) | (Qiu et al., 2017) | http://cole-trapnell-lab.github.io/ |
| | | monocle-release/ |
| GO (David 6.7) | Huang et al., 2009 | https://david-d.ncifcrf.gov |
| Cell Ranger (2.2.0) | NA | https://support.10xgenomics.com/ |
| | | single-cell-gene- |
| | | expression/software/pipelines/ |
| | | latest/what-is-cell-ranger |
| Cluster 3.0 | NA | http://bonsai.hgc.jp/* mdehoon/ |
| | | software/cluster/software.htm |
| WGCNA (1.68) | (Langfelder and | https://horvath.genetics.ucla.edu/ |
| | Horvath, 2008) | html/CoexpressionNetwork/ |
| | | Rpackages/WGCNA/Tutorials/ |
| Cytoscape (3.7.2) | (Otasek et al., 2019) | https://cytoscape.org |
| Other | | |
| Single cell RNA-seq for infant and | Guo et al., 2018 | GEO: GSE120508 |
| adult human testes | | |
| Single cell RNA-seq for neonatal | Sohni et al., 2019 | GEO: GSE124263 |
| human testes | | |

### Experimental Model and Subject Details

Prenatal male gonads from 6 to 16 weeks post-fertilization were obtained from three collaborating laboratories at University of Washington Birth Defects Research Laboratory (BDRL), University of Tubingen and Karolinska Institutet. At BRDL, the prenatal gonads were obtained with regulatory oversight from the University of Washington IRB approved Human Subjects protocol, combined with a Certificate of Confidentiality from the Federal Government. The research project was also approved by the research ethics committee of the University of Tubingen. All consented material was donated anonymously and carried no personal identifiers. Human first trimester tissue was collected after elective surgical terminations with maternal written informed consent. The Regional Human Ethics Committee, Stockholm, Sweden, approved the collection (Dnr 2007/1477-31 with complementary permissions 2011/1101-32 and 2013/564-32. The ethical approval to perform the gonadal studies: Dnr 2013/457-31/4). Developmental age was documented by BDRL and University of Tu€bingen as days post fertilization using a combination of prenatal intakes and Carnegie staging. Developmental age was documented by Karolinska Institutet as days post fertilization by the examination of anatomical landmarks such as nervous system, limb, eye and gonadal development according to the atlas of England. Formalin fixed and paraffin embedded adult testis from biobank samples without underlaying testicular pathologies was obtained at the Department of Pathology at the Karolinska Institutet, and Karolinska University Hospital (ethical approval: Dnr 2014/267-31/4).

Postnatal human testicular sample (5 months old) was obtained through the University of Utah Andrology laboratory and Donor-Connect. This sample was removed from deceased individuals who consented to organ donation for transplantation and research.

### Method Details

### Sample Transportation and Storage

The prenatal samples collected at BDRL used for single cell transcriptome profiling were shipped overnight in HBSS with an ice pack for immediate processing in Los Angeles. From University of Tu€bingen samples were delivered to UCLA within 24-48 hours after the procedure.

The postnatal whole testis was transported to the research laboratory on ice in saline and processed within 1 hour of removal by surgery. Around 90% of each testis was divided into smaller portions (·500 mg—1 g each) using scissors and directly transferred into cryovials (Corning cat #403659) in DMEM medium (Life Technologies cat #11995073) containing 10% DMSO (Sigma-Aldrich cat #D8779), 15% fetal bovine serum (FBS) (GIBCO cat #10082147) and cryopreserved in Mr. Frosty container (Thermo Fisher Scientific cat #5100-0001) at a controlled slow rate, and stored at −80° C. for overnight. Cryovials were transferred to liquid nitrogen for longterm storage.

### Human Testis Sample Preparation for Single Cell RNA Sequencing

Prenatal tissues were processed within 24-48 hours after termination. Upon arrival to UCLA tissues were gently washed with PBS and placed in dissociation buffer containing collagenase IV 10 mg/ml (Life Technologies #17104-019), Dispase II 250 ug/ml (Life Technologies #17105041), DNase I 1:1000 (Sigma 4716728001), 10% FBS (Life Technologies 10099141) in 1×PBS. After every 5 minutes tissues were gently pipetted with P1000 pipette against the bottom of Eppendorf tube. This process was repeated 3 times for a total of 15 minutes. Afterward, cells were centrifuged for 5 minutes at 500 g and pellet was resuspended in 1×PBS with 0.04% BSA and strained through 40 mm strainer and counted using automated cell counter (Thermo Fisher, Countess II). The cell concentration was adjusted to 800-1200 cells per microliter and immediately used for scRNA-seq. For postnatal tissues, 1 cryovial of tissue was thawed quickly, which was then washed twice with PBS, and subject to digestion as described previously (Guo et al., 2018). Tissues were washed twice in 1×PBS and minced into small pieces for better digestion outcome. Tissues were then treated with trypsin/ethyl-enediaminetetraacetic acid (EDTA; Invitrogen cat #25300054) for 20-25 min and collagenase type IV (Sigma Aldrich cat #C5138-500MG) at 37° C. Single testicular cells were obtained by filtering through 70 mm (Fisher Scientific cat #08-771-2) and 40 mm (Fisher Scientific cat #08-771-1) strainers. The cells were pelleted by centrifugation at 600 g for 15 min and washed with PBS twice. Cell number was counted using a hemocytometer, and the cells were then resuspended in PBS+0.4% BSA (Thermo Fisher Scientific cat #AM2616) at a concentration of 1,000 cells/uL ready for single-cell sequencing.

### Single Cell RNA-Seq Performance, Library Preparation and Sequencing

It was targeted to capture 6,000-7,000 cells. The prenatal sequencing was conducted in UCLA, and the postnatal sequencing was conducted at University of Utah. Briefly, cells were diluted following manufacturer's instructions, and 33.8 mL of total mixed buffer together with cells were loaded into 10× Chromium Controller using the Chromium Single Cell 3′ v3 reagents. The sequencing libraries were prepared following the manufacturer's instructions, using 13 cycles of cDNA amplification, followed by an input of 100 ng of cDNA for library amplification using 12 cycles. The resulting libraries were then sequenced on a 2×150 cycle paired-end run on an Illumina Novaseq 6000 instruments.

### Processing of Single Cell RNA-Seq Data

Raw data were demultiplexed using mkfastq application (Cell Ranger v2.2.0) to make Fastq files. Fastq files were then run with count application (Cell Ranger v2.2.0) using default settings, which performs alignment (using STAR aligner), filtering and UMI counting. The UMI count tables were used for further analysis.

### Immunostaining of Testicular Tissues

Intact testes were fixed in 4% PFA at room temperature for 2 hours on a platform rocker. Tissues were washed 3 times with PBS for 10 minutes each wash then placed into paraffin blocks (Histogel, Thermo Scientific HG4000012) for sectioning onto slides. Sections were deparaffinized and rehydrated in a Xylene then ethanol series (100%, 95%, 70%, 50%, water) respectively. Antigen retrieval was performed in either Tris-EDTA solution (pH 9.0) or Sodium Citrate Solution (pH 6.0) in a hot water bath (95° C.) for 40 minutes. Sections were washed in PBS, 0.2% Tween-20 (PBS-T) 3 times, 5 minutes each then permeabilized in PBS, 0.05% Trition X-100 for 20 minutes. Sections were blocked with blocking solution (10% Normal Donkey Serum (NDS), PBS-T) for 30 minutes at room temperature in a humid chamber. Primary Antibodies were diluted in 2.5% NDS, PBS-T at the appropriate dilutions (see Key resources table) and incubated overnight at 4° C. in a humid chamber. After 3 washes in PBS-T (5 minutes each) secondary antibodies were added and allowed to incubate at room temperature for 1 hour in a humid chamber. After 3 washes in PBS-T, DAPI was added to the sections for approximately 5 minutes, then washed 3 times 5 minutes each in PBS-T. Prolong Gold antifade mountant (Invitrogen P10144) was added to the sections. Coverslips were placed onto slides then sealed with nail polish. Slides were allowed to cure overnight, in the dark, at room temperature then subsequently stored at 4° C. until ready to image. For sections stained with PIWIL4 antibody, the blocking buffer used was Superblock blocking buffer (Thermo Scientific 37580). In addition, the SignalBoost Immunoreaction Enhancer Kit (Millipore 407207) was used to dilute primary and secondary antibodies for experiments involving PIWIL4 antibody.

### Microscopy

A Zeiss LSM 880 with Airyscan controlled by the Zen Black software, equipped with the Plan-Apochromat 203/0.8 NA and the Plan-Apochromat 633/1.4 NA M27 oil immersion objective, was used to acquire confocal images. Saved CZI files were converted to Imaris format files (.ims) using the Imaris File converter (Bitplane), then processed using the image analysis software IMARIS 9.3 (Bit-plane). An Olympus BX-61 light microscope was used to examine Hematoxylin and Eosin (H&E) stained slides. The ImageJ stitch function uses similar features/structures from a collection of images to make a fused image, therefore each image has some overlap with the previous image taken. Briefly, H&E images were taken with the 20× objective. In ImageJ under the Plugins dropdown box the Stitching plugin was chosen and then selected the Grid/Collection Stitching function. In the “Type” box “unknown position” was selected and “all files in directory” was chosen for the “Order”. Linear Blending was chosen for the Fusion Method used. The Regression threshold was set at 0.30. The Max/avg displacement threshold was set at 2.50 and the Absolute displacement threshold was set to 3.50. Stitched images were built using the ImageJ2(NIH) Grid/Collection Stitching plugin.

### Quantification and Statistical Analysis

The Seurat program was used as a first analytical package. To start with, UMI count tables from both replicates from all four juvenile donors were loaded into R using Read10× function, and Seurat objects were built from each experiment. Each experiment was filtered and normalized with default settings. Specifically, cells were retained only if they contained >500 expressed genes and had <25% reads mapped to mitochondrial genome. t-SNE and clustering analysis were first run on each replicate, which resulted in similar t-SNE map. Data matrices from different donors and replicates were then combined with the previously published infant and adult data (Guo et al., 2018). Next, cells were normalized to the total UMI read counts, as instructed in the tutorial. t-SNE and clustering analyses were performed on the combined data using the top 6,000 highly variable genes and 1-30 PCs, which showed the most significant p values.

Detailed pseudotime for different cell types were performed using the Monocle package (v2.10.1) following the default settings. After pseudotime coordinates/order were determined, gene clustering analysis was performed to establish the accuracy of pseudo-time ordering. Here, cells (in columns) were ordered by their pseudotime, and genes (in rows) were clustered by k-means clustering using Cluster 3.0. Different k-mean numbers were performed to reach the optimal clustering number. Cell cycle analysis was performed using scran program R Package; v1.6.5).

### Weighted Correlation Network Analysis

Hub genes in PGC, spermatogonia and State 0 were found by WGCNA. When finding hub genes in PGC and spermatogonia, gene expression data of 40 cells from PGC and State 0 respectively were randomly extracted from the UMI count tables of scRNA-seq data. Genes were filtered by selecting those genes expressed in more than 20 cells since scRNA-seq data had a high drop-out rate and low expression genes may represent noise. Then the counts were normalized by total reads (x*100000/total reads) and then log-transformed (log 2(x+1)). Afterward, one-step network construction and module detection were performed. In this step, parameters including signed hybrid network type, Pearson correlation method and the default soft-threshold power b were chosen to reach the scale-free network topology. To identify the modules that were significantly correlated with PGC or spermatogonia, bi-weight mid-correlation (robustY=FALSE) was used. The quality of the modules was checked by the strong correlation between module eigengenes and traits of interest as well as the strong correlation between gene module membership and gene-trait correlation. Finally, hub genes inside those modules were selected from the top 40 genes with the highest intramodular connectivity (sum of in-module edge weights). Specifically, in order to find hub genes in State 0 rather than spermatogonia, gene expression data of 40 cells from State1 was added to rule out the genes expressing broadly in States 0-4 and performed the same analysis to determine the modules that were significantly correlated with State 0. Ten hub genes were selected by the same standard. Finally, the networks were visualized by Cytoscape Software 3.7.2.

### Example 2. Establishing a Human Testicular Tissue Culture System

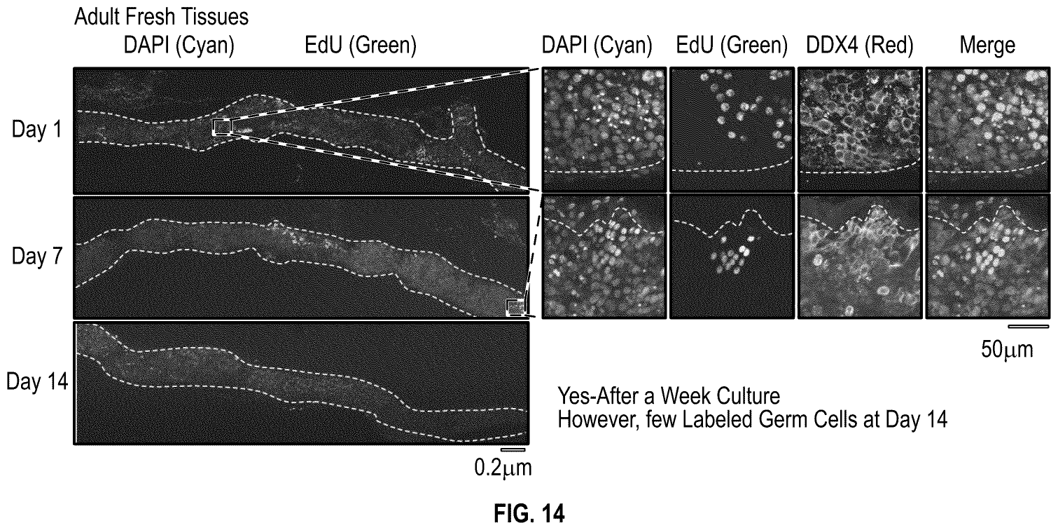

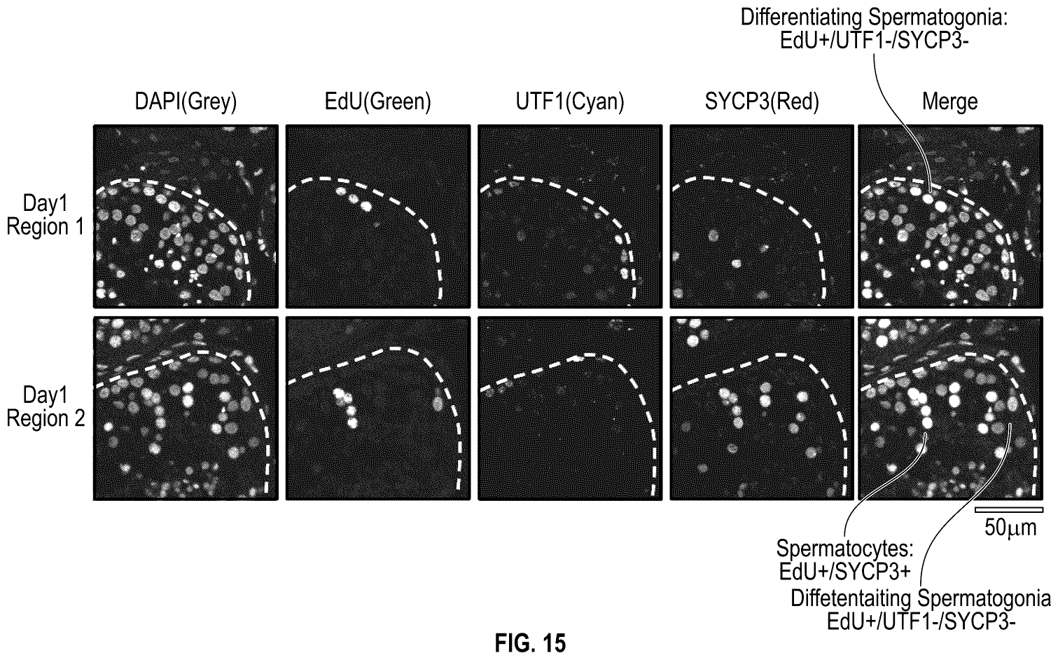

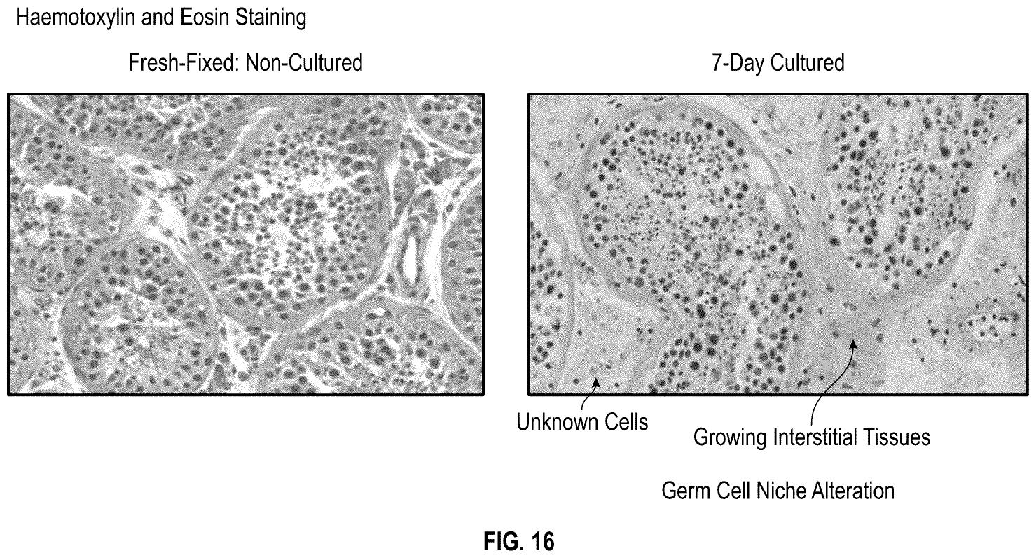

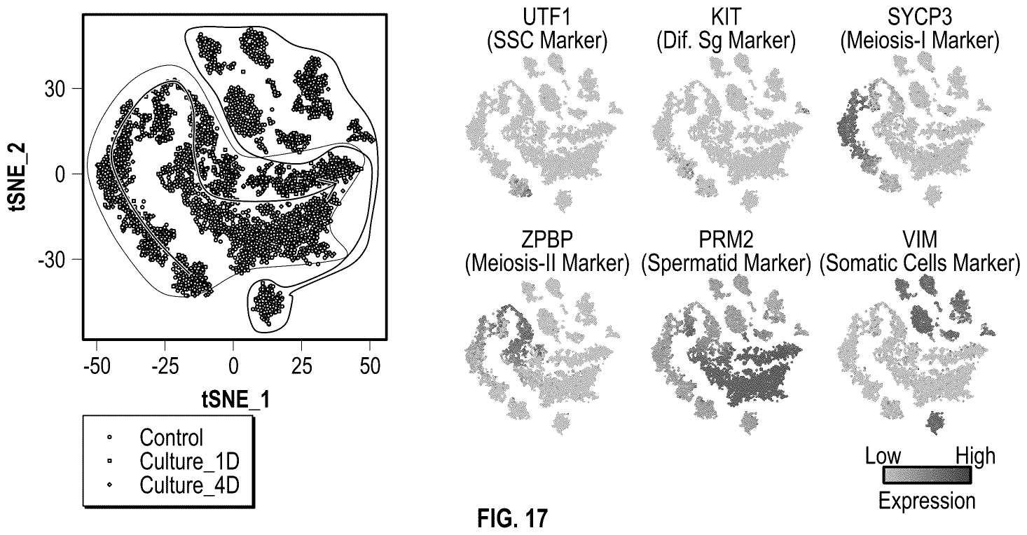

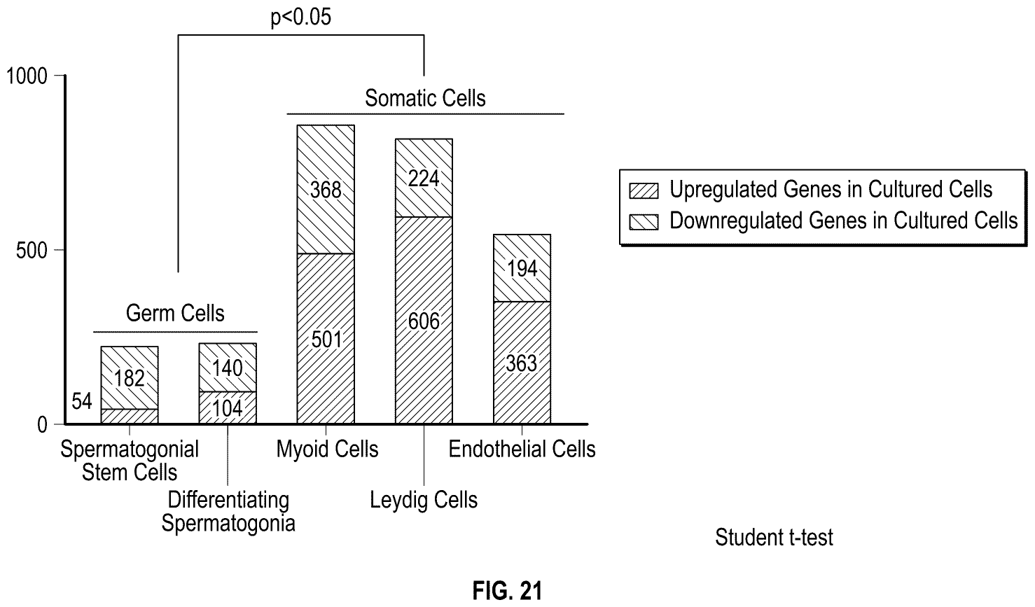

To identify culture conditions that support growth and development of testicular germ cells in vitro, both germline and somatic, a genomic approach was used to identify dysregulated biological pathways in cultured testicular tissue comprising tubules and germ cells that can be used to identify the in vitro culture conditions. Using tissue analysis approach to analyze in vitro cultured tissue, it was discovered that spermatogonia were able to proliferate/replicate in vitro and differentiating spermatogonia were able to proliferate/replicate and enter meiosis (FIGS. 14 and 15). However, although germ cells proliferate under base culture conditions, very few cells were found 14 days after start of culture. Using an immunohistochemical approach, it was discovered that the germ cell niche was altered after 7 days of culture. More specifically, results show that somatic cells rather than germ cells display most alteration after culturing (FIGS. 16, 17 and 21).

To identify dysregulated pathways in cultured tissue, a process using a genomic approach using scRNA-seq was used to further reveal the molecular changes of cultured testicular tissue using a process detailed in the diagram shown in FIG. 25. Testicular tissue comprising seminal tubules was obtained from healthy adult subjects and cultured under basic culture conditions using the methods described below. The cells were dissociated to obtain single cells of all types of testicular types. The RNA transcripts in each cell type in cultured cells was compared to the level of RNA transcripts in the corresponding cells dissociated from tissue obtained directly from a subject. In this instance, the single-cell RNA sequencing transcriptome profile of the adult human testis atlas (Guo, et al. 2018 Cell Research 28, 1141; the disclosure of all of which is incorporated herein in its entirety), which provides a comprehensive characterization of cell types in the human adult testis.

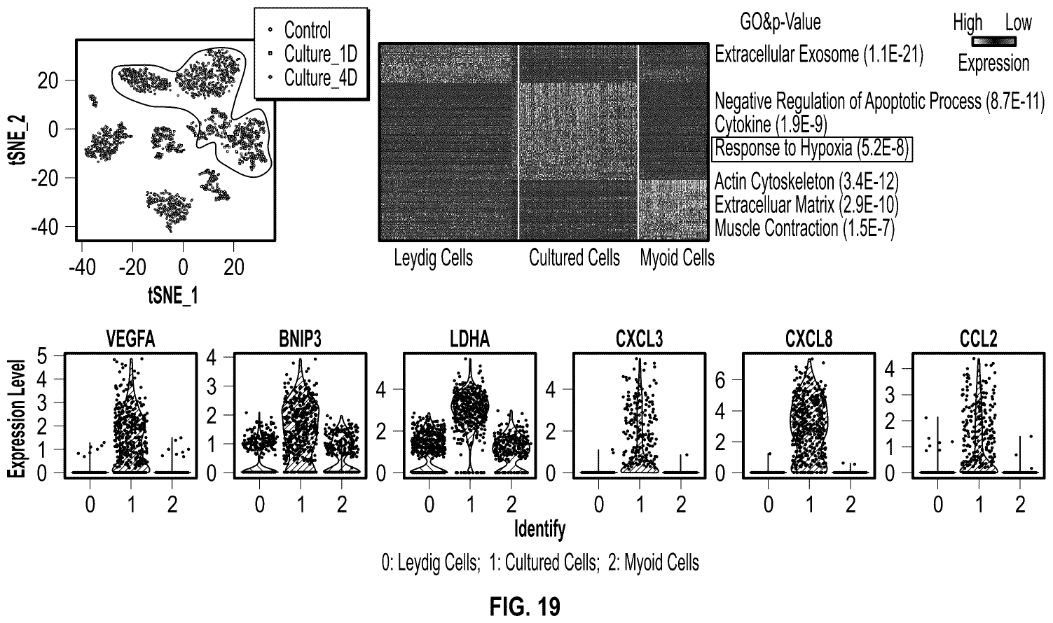

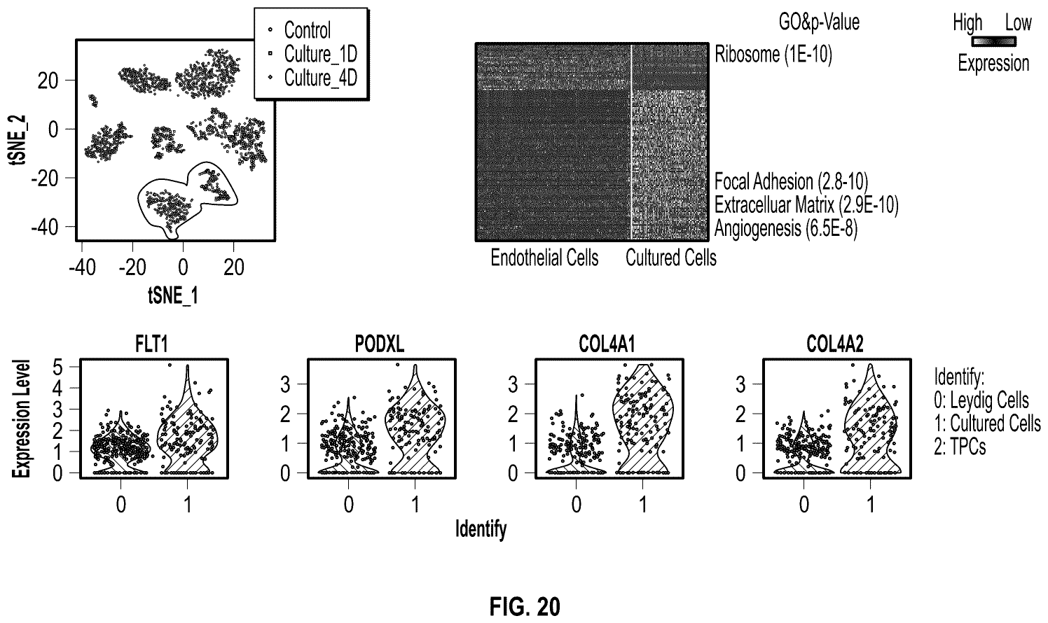

Using the genomic approach described above, it was discovered that expression of the genes associated with the following were altered in cultured Leydig and myoid cells: extracellular exosome, negative regulation of apoptotic process, cytokine, response to hypoxia, actin cytoskeleton, extracellular matrix, and muscle contraction (FIG. 19). It was also discovered that expression of the genes associated with the following were altered in cultured endothelial cells: ribosome, focal adhesion, extracellular matrix, and angiogenesis (FIG. 20).

Among the dysregulated pathways, it was discovered that the HIF pathway was activated in somatic cells of cultured testicular tissue. Dysregulation of the HIF pathway and genes having altered expression in the somatic cells of cultured tissue are shown in FIG. 26. Genes associated with the following pathways affected by dysregulation of the HIF pathway were altered in cultured Leydig and myoid cells: extracellular exosome, negative regulation of apoptotic process, cytokine, response to hypoxia, actin cytoskeleton, extracellular matrix, and muscle contraction.

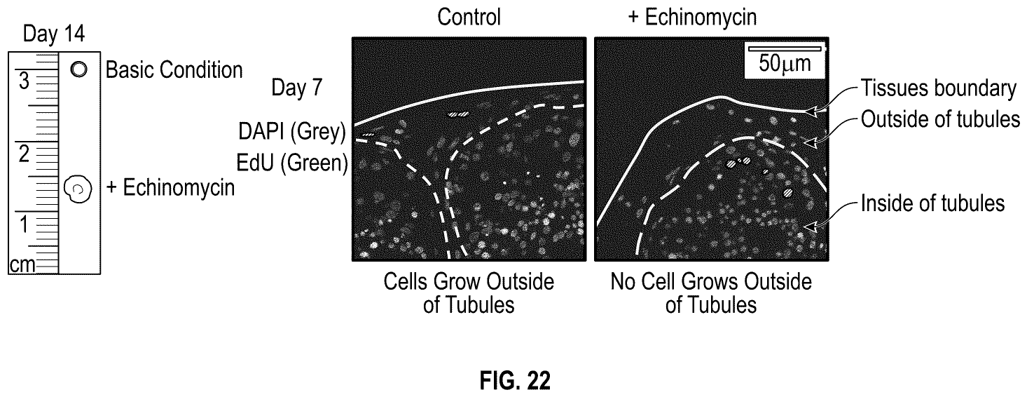

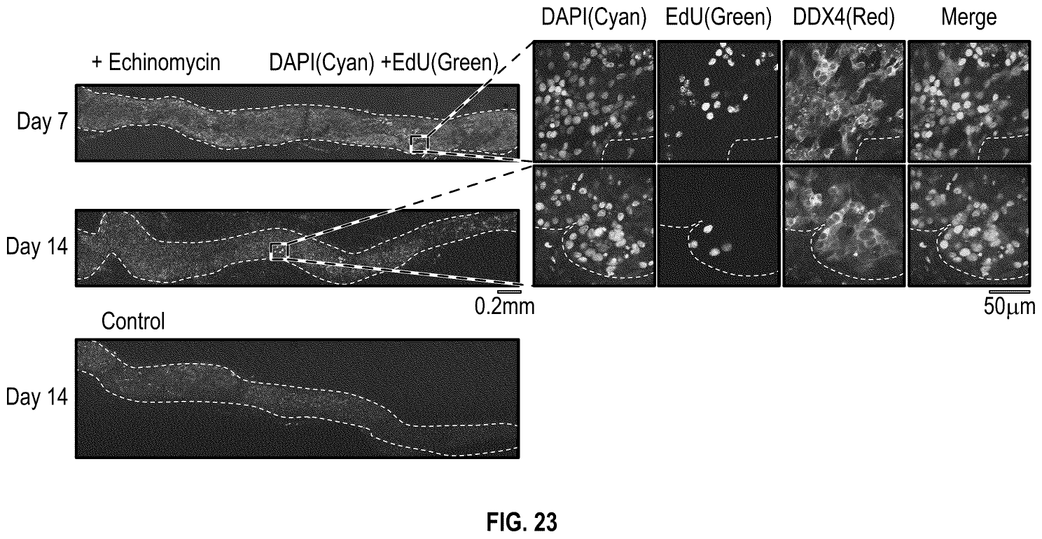

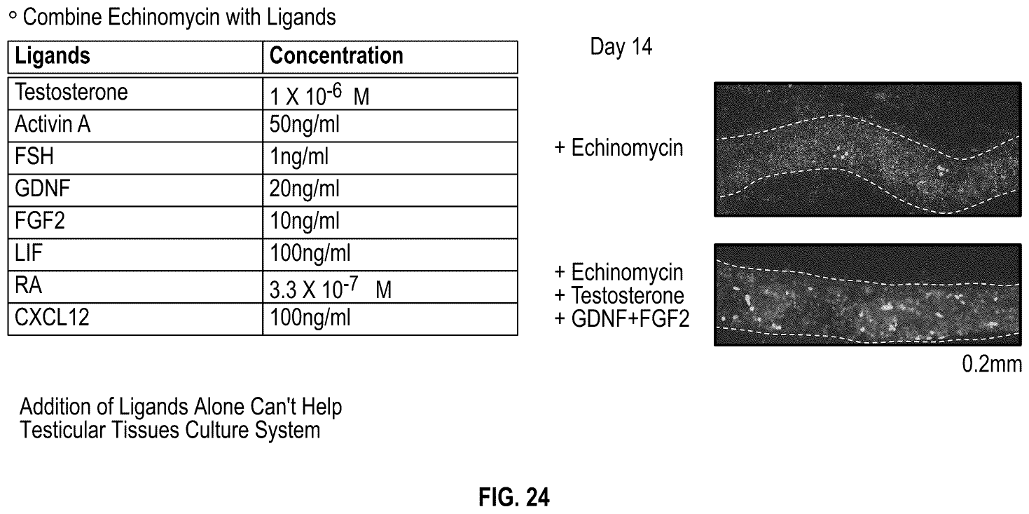

Considering the above, small molecule inhibitors (Table 1) were used against some pathways in the HIF pathways to determine if and which inhibitor can reverse the effects of culturing and maintain testicular tissue structure. To accomplish this, the iterative method of identifying factors that could improve culture conditions was used to test the effect of the small molecule inhibitors described in Table 1 with parameters described further below. It was discovered that the HIF-1 inhibitor echinomycin helps maintain testicular tissue structure (FIGS. 22, and 23) and helps germ cell survival (FIG. 24) even two weeks after start of culture. Accordingly, the results show that the cultured somatic cells are under low oxygen tension and demonstrate the effectiveness of the described process at identifying factors that improve culture conditions for culturing testicular germ cells.

TABLE 1
Small molecule inhibitors.

| | Name | function |
|---|---|---|
| Hypoxia | Echinomycin | HIF-1 inhibitor |
| | PX-12 | HIF-1 inhibitor |
| | Vitexin | HIF-1 inhibitor |
| | 1400W | NOS inhibitor |
| | Isoliquiritigenin | NLRP3 Inflammasome |
| | | Inhibitor |
| | Glybenclamide | NLRP3 Inflammasome |
| | | Inhibitor |
| | Celecoxib | COX2 inhibitor |
| | Semapimod hydrochloride | Macrophage inhibitor |
| | Acetylsalicylic acid | Inflammation inhibitor |
| | Ibuprofen | Inflammation inhibitor |
| | Interleukin-1 Receptor | IL1R inhibitor |
| | Antagonist | |
| | Caffeic Acid Phenethyl | NF-kB inhibitor |
| | Ester | |
| ROS | N-acetylcysteine amide | ROS scavenger |
| | Melatonin | ROS scavenger |
| | Trolox | ROS scavenger |
| | Pazopanib | VEGF inhibitor |
| | Tranlist | Angiogenesis inhibitor |
| | Dasatinib | PDGFR inhibitor |
| | Nintedanib | VEGFR inhibitor |
| Fibrosis | PXS-5153A | LOXL2/3 inhibitor |
| | SIS3 | Smad3 inhibitor |
| | Pirfenidone | TGF-b inhibitor |
| | Itraconazole | Fibrosis inhibitor |

Culture media was also supplemented with ligands (Table 2) informed by the genomic work here and previously discovered by the inventors (Guo et al., Cell Stem Cell, 2017; Guo et al., Cell Research, 2018; and Guo et al., Cell Stem Cell, 2020).

TABLE 2

| Ligands | Concentration | |
|---|---|---|
| Testosterone | 1 × 10⁻⁶M | |
| Activin A | 50 | ng/ml |
| FSH | 1 | ng/ml |
| GDNF | 20 | ng/ml |
| FGF2 | 10 | ng/ml |
| LIF | 100 | ng/ml |
| RA | 3.3 × 10⁻⁷M | |
| CXCL12 | 100 | ng/ml |

It was discovered that the ligands were effective but not sufficient for restoring tissue structure when echinomycin was not also added. Conversely, when echinomycin, testosterone, FSH, and RA are added to the medium, resulted in better germ cell proliferation when compared to echinomycin alone or ligands alone (FIG. 24).

### Testicular Tissue Culture

### Materials

Petri dish (Genesee Scientific #32103)
CytoOne 6-well TC plate (USA scientific #CC7682-7506)
αMEM (STEMCELL Technologies #36453)
KnockOut™ Serum Replacement (KSR; Gibco #10828010)
Echinomycin (Millopore Sigma #SML0477)
Testosterone (empower pharmacy #49696)
GDNF (recombinant human glial cell line-derived neurotrophic factor; R&D systems #212-GD-010)
bFGF (basic fibroblast growth factor; BD Biosciences #354060-10)
Click-iT Edu (Thermo Fisher Scientific #C10337)
Collagenase type IV (Sigma Aldrich cat #C5138-500MG)

### Method

Tissue preparation: Whole testes are removed from cadaveric organ donors by DonorConnect staff, which are picked up by Utah team and transported to research lab on ice. Testicular tissues are cut by 3-5 mm by dimeter in size using surgical scissors and tweezers.

Culture media preparation: the base media is αMEM+10% KSR. We first tested various small molecular inhibitors/ligands with different concentrations in the base media to culture testicular tissues. We then chose to use the inhibitor/ligand combinations with better outcomes based on morphology change and germ cell proliferation status (i.e. maintained testicular size with germ cell proliferation; see below for more details). One of the most effective combination of proliferation factors we current have to date is echinomycin (HIF inhibitor; concentration: 5 nM)+Testosterone (concentration: 10-7M)+GDNF (concentration: 10 ng/mL)+bFGF (concentration: 10 ng/mL).

Tissue culture: Place three pieces of testicular tissue into one well of a 6-well plate with 2 ml of media in each well. The tissue should be fully immersed in media. Place the plate at 34° C. in 5% CO2 in an incubator with culture media changed every other day.

Morphology examination: Measure the sizes of the cultured testicular tissues at different time points, including Day 1, Day 7, Day 14, and Day 21. And then fix the sample for H&E staining. The detailed method has been described previously (Guo et al., 2018, 2020).

Germ cell proliferation examination: Add Edu (2 ul into each well) at different time points at Day 0, Day 6, Day 13, Day 20. Harvest the tissue 24 hours later for the germ cell proliferation test. Samples are washed three times by PBS and digested to isolated tubules by collagenase type IV at 37° C. Then wash 3 times by PBS to terminate the digestion and perform whole-mount staining of the tubules with Edu and DDX4/UTF1/SYCP3. The detailed method has been described previously (Gassei et al., 2014).

Single cell-RNA seq profiling of cultured tissues: Single cell transcriptome of the cultured testicular tissues in the most effective combination is obtained. The detailed method for tissue dissociation and sequencing execution has been described previously (Guo et al., 2018, 2020). We make comparisons of the cultured profile with non-cultured healthy testicular profiles, which allows us to refine our culture media by testing more small molecular inhibitors/ligands.

### Parameters

Extract Testicular Somatic Cells for tSNE/UMAP Analysis
tsne.method
   tsne.method=“tSNE: Use the Rtsne package Barnes-Hut implementation of tSNE
   tsne.method=“Flt-SNE”: Use the FFT-accelerated Interpolation-based t-SNE
reduction (dimensional reduction)
   PCA or ICA

### Differential Expression Analysis in Each Cell Type

Tests used to identify differentially expressed genes:
   test.use=“wilcox”: Wilcoxon Rank Sum test
   test.use=“ROC”: ROC analysis
   test.use=“t”: Student's t-test
   test.use=“negbinom”: method based on a negative binomial generalized linear model
   test.use=“DESeq2”: DESeq2 analysis which uses a negative binomial distribution
   test.use=“poisson”: method based on a poisson generalized linear model
   test.use=“MAST”: method based on a hurdle model tailored to scRNA-seq data
min.pct (genes that are detected in a minimum fraction of min.pct cells) min.pct setting: from 0.1 to 0.5
logfc.threshold (limit genes to at least X-fold difference (log-scale)) logfc.threshold setting: from 0.25 to 0.5

TABLE 3
GO terms of genes in heat map of FIG. 19

| | Negative | | | | | |
|---|---|---|---|---|---|---|
| | regulation | | | | | |
| Extracellular | of apoptotic | Response | Response | Actin | Extracelluar | Muscle |
| exosome | process | to Cytokine | to hypoxia | cytoskeleton | matrix | contraction |
| RPL13A | CA9 | VEGFA | LOXL2 | ABLIM1 | ENG | IGF2 |
| RPL27A | PLOD2 | TGFB3 | KCNK3 | CALD1 | ELN | CALD1 |
| RPL31 | VEGFA | TFRC | CA9 | DSTN | MYH11 | CALM2 |
| RPS20 | TGFB3 | TGFB1 | PLOD2 | GSN | IGFBP7 | ENG |
| RPS17 | TFRC | SMAD3 | VEGFA | LMOD1 | SPARCL1 | GSN |
| RPL38 | AK4 | PTGIS | TGFB3 | MYH11 | LTBP4 | MYL9 |
| RPL35A | TGFB1 | PDGFRB | ANGPTL4 | MYLK | FBLN5 | FXYD1 |
| RPL7 | ACAA2 | ADM | STC1 | NEXN | MGP | TPM1 |
| RPL27 | PGK1 | HIF1A | TFRC | PALLD | SMOC2 | FHL2 |
| RPS29 | SMAD3 | PTGS2 | MMP14 | SPTBN1 | DCN | LMOD1 |
| RPS15A | PTGIS | BNIP3 | AK4 | TMSB4X | PODN | IGF1 |
| RPS27 | PDGFRB | GGT5 | TGFB1 | TAGLN | COL1A1 | SOD1 |
| RPL34 | ADM | SERPINE1 | P4HB | TPM1 | SPARC | SCN7A |
| RPS16 | HIF1A | IGFBP3 | HSP90B1 | TPM2 | COL1A2 | FXYD6 |
| RPL37A | PTGS2 | TIMP1 | HILPDA | ACTA2 | LAMA2 | ELN |
| RPS23 | ATP6 | CD44 | ERO1A | MYL9 | OGN | ACTA2 |
| RPL3 | BNIP3 | GAPDH | ACAA2 | TAGLN | FLRT2 | SSPN |
| FAU | SLC16A3 | IL6 | PGK1 | AHNAK | COL15A1 | MYH11 |
| RPL9 | GGT5 | MIF | PLOD1 | TAX1BP3 | LAMB2 | GAMT |
| RPL21 | LIPG | ATP1A2 | SMAD3 | EZR | CILP | PDGFRB |
| RPL26 | SERPINE1 | ANGPTL4 | PTGIS | ANXA6 | FBLN2 | ANXA6 |
| RPS15 | SCD | STC1 | MT3 | | ECM2 | PLD3 |
| RPL30 | SLC16A1 | MMP14 | COL1A1 | | OMD | SPRY1 |
| RPS8 | SLC39A14 | P4HB | PDGFRB | | MFGE8 | MYLK |
| RPS25 | IGFBP3 | HSP90B1 | CFLAR | | LAMC3 | TPM2 |
| RPS27A | TIMP1 | MT3 | ADM | | IGFBP6 | |
| RPS13 | SLC6A8 | COL1A1 | HIF1A | | PRELP | |
| RPS4X | GBE1 | CFLAR | SOD2 | | CAV1 | |
| RPLP2 | LYVE1 | SOD2 | PTGS2 | | MFAP4 | |
| RPL24 | GYS1 | DDIT4 | ATP6 | | DPT | |
| RPLP1 | CD44 | PGF | DDIT4 | | VIT | |
| RPL15 | FMOD | MMP2 | BNIP3 | | PBXIP1 | |
| RPL23A | ALDH1A3 | SFRP1 | PGF | | NID1 | |
| RPL32 | NT5E | TREM1 | MMP2 | | MMP23B | |
| COX7C | ENO1 | LOXL3 | SFRP1 | | COL3A1 | |
| RPL13 | PTGES | MME | | | QSOX1 | |
| RPL23 | GAPDH | COL7A1 | | | CTSK | |
| RPL22 | PFKL | NRP1 | | | CCN2 | |
| RPL37 | VCAN | CSF3 | | | BGN | |
| RPL41 | MSMO1 | CXCL5 | | | A2M | |
| RPS11 | PLIN2 | SSC5D | | | | |
| RPL36A | COX3 | IL11 | | | | |
| IGFBP7 | CHSY1 | EGFL7 | | | | |
| SKP1 | COX2 | COL3A1 | | | | |
| RPL36AL | GANAB | LIF | | | | |
| INMT | ND6 | PDIA3 | | | | |
| UBB | LDHA | CXCL1 | | | | |
| RPL10 | COX1 | IGFBP4 | | | | |
| RPS21 | PGAM1 | IL33 | | | | |
| EIF4A2 | ACADVL | ITGB1 | | | | |
| CKB | IGFBP2 | LOX | | | | |
| COX4I1 | IL6 | FZD4 | | | | |
| NAP1L1 | BGN | CALR | | | | |
| RPL18A | MIF | CD68 | | | | |
| TXNIP | ATP1A2 | CXCL8 | | | | |
| RAC1 | | BDKRB2 | | | | |
| RPL39 | | CXCL3 | | | | |
| SOD1 | | FN1 | | | | |
| DPEP1 | | ECM1 | | | | |
| HSPA1A | | OSMR | | | | |
| RWDD1 | | PLAUR | | | | |
| ABLIM1 | | IRAK3 | | | | |
| ZFP36 | | ANGPTL2 | | | | |
| RPL17 | | ECE1 | | | | |
| CRYAB | | ICAM1 | | | | |
| GPX3 | | NIBAN2 | | | | |
| H3-3B | | HSPA5 | | | | |
| PIK3R1 | | CCL2 | | | | |
| EIF1 | | MT2A | | | | |
| PMP22 | | DDOST | | | | |
| HSPA1B | | PPIB | | | | |
| SCN7A | | EDNRB | | | | |
| FKBP5 | | TNFRSF12A | | | | |
| HSPB1 | | TFPI | | | | |
| PLPP3 | | CXCL6 | | | | |
| EZR | | AKAP12 | | | | |
| RGMA | | ACTG1 | | | | |
| C7 | | FGF7 | | | | |
| MYH11 | | TUBA1B | | | | |
| FOS | | | | | | |
| ADI1 | | | | | | |
| PLPP1 | | | | | | |
| LAMA2 | | | | | | |
| HSP90AA1 | | | | | | |
| H4C3 | | | | | | |
| DNAJA1 | | | | | | |
| MYL9 | | | | | | |
| CFD | | | | | | |
| DNAJB1 | | | | | | |
| JUN | | | | | | |
| MT1A | | | | | | |
| PEMT | | | | | | |
| ACTA2 | | | | | | |
| HSPH1 | | | | | | |
| ATF3 | | | | | | |

TABLE 4
GO terms of genes in heat map of FIG. 20.

| | Focal | Extracelluar | |
|---|---|---|---|
| Ribosome | adhesion | matrix | Angiogenesis |
| RPL34 | MMP14 | MMP14 | ACKR3 |
| RPS17 | P4HB | ITGB1 | PGF |
| TXNIP | BMP2 | ADAMTS9 | PLAU |
| RPS20 | PDGFA | ADAMTS1 | PNP |
| RPL31 | LAMA4 | P4HB | PLAUR |
| RPL27A | SERPINE1 | BMP2 | FMNL3 |
| RPL13A | THBS1 | IGFBP3 | GJA1 |
| ID1 | NID1 | PDGFA | DLL4 |
| IGFBP7 | ITGA5 | LAMB1 | GAPDH |
| RPS15A | IGFBP2 | ADAMTS4 | TUBA1C |
| RPL38 | PLAU | LAMA4 | ACTG1 |
| RPLP2 | PNP | NID2 | MMP14 |
| RPS27 | PLAUR | COL18A1 | CLIC1 |
| RPL7 | ACTG1 | PXDN | ITGB1 |
| ASS1 | CALR | COL4A1 | ADAMTS9 |
| RPL35A | CXCL8 | ERO1A | HBEGF |
| RPS29 | VCL | PECAM1 | RAP1B |
| RPL27 | ETS1 | SERPINE1 | ADAMTS1 |
| RPS23 | CXCR4 | ADAMTS5 | CCND1 |
| RPL23A | CD200 | THBS1 | CALR |
| RPS25 | SPRY4 | COL4A2 | RHOC |
| RPL37 | CD276 | PLOD1 | DUSP5 |
| RPL39 | THY1 | NID1 | P4HB |
| RPL36A | MYADM | CTHRC1 | CALU |
| RPL3 | CD81 | HSPG2 | BMP2 |
| RPS14 | TNFRSF4 | EXT1 | CXCL8 |
| RPS21 | SOX4 | PPIB | PGK1 |
| VWF | LGALS1 | SPP1 | MAPKAPK2 |
| RPL9 | RGCC | LAMC1 | EIF4G2 |
| RPL36 | PODXL | ITGA5 | FLT1 |
| RPL32 | CD9 | CCN3 | PDIA6 |
| RPS19 | ARHGEF7 | IGFBP2 | VCL |
| RPL37A | MIF | LOXL2 | CFL1 |
| COX7C | PPM1F | ESM1 | FSCN1 |
| RPL41 | NOTCH1 | ICAM2 | TKT |
| RPL21 | JUP | ITGA6 | PFN1 |
| RPL35 | ARPC2 | TIMP1 | ETS1 |
| RPS4X | PLPP3 | SERPINH1 | HDAC1 |
| RPS18 | YES1 | | LMAN1 |
| RPL17 | MAP4K4 | | IGFBP3 |
| HIF3A | CD55 | | CRIP2 |
| EPAS1 | ANGPT2 | | HSP90AA1 |
| RPL12 | EFNB1 | | SOD2 |
| INMT | PRELID1 | | PRRC2C |
| ZC3H11A | JUND | | MYH9 |
| ARL6IP5 | ADGRF5 | | LDHA |
| APOA1 | ZEB1 | | DNAJA1 |
| ACACB | MACF1 | | |
| ZFP36 | TGM2 | | |
| PLAAT4 | EFNB2 | | |
| HSPB1 | PKN1 | | |
| FKBP5 | FERMT2 | | |
| ENAH | APOD | | |
| MT1X | FLNA | | |
| MT1E | | | |
| MT1M | | | |
| MT2A | | | |

### Example 3. Process for Identifying SSC Ligands

A process for identification and selection of receptor ligands capable of improving testicular germ cell proliferation was developed. The process uses expression data obtained by mining scRNA-seq data obtained from testicular tissue (FIG. 27). The process systematically prioritizes candidates for spermatogonia ligands through a series of bioinformatics analyses and filtering. The process comprises identifying and selecting potential ligands based on their receptor expression in State 1 SSCs (active stem cells) and State 2/3 differentiating spermatogonia (differentiated germ cells with strong proliferation ability), or any combination thereof.

More specifically, the approach was based on the single-cell RNA sequencing transcriptome profile of the adult human testis atlas (Guo, J., Grow, E. J., Mlcochova, H., Maher, G. J., Lindskog, C., Nie, X., Guo, Y., Takei, Y., Yun, J., Cai, L., et al. (2018). The adult human testis transcriptional cell atlas. Cell Research 28, 1141. 10.1038/s41422-018-0099-2; the disclosure of all of which is incorporated herein in its entirety), which provides a comprehensive characterization of cell types in the human adult testis. Pseudo-bulk RNA-seq analysis was used, which involves aggregating individual cells into groups to enhance the receptor gene expression signal on SSCs and differentiating spermatogonia. Each individual cell is assigned to its corresponding cell type cluster based and summing the expression values of all cells within each cluster to create pseudo-bulk samples.

The CellTalkDB database was used to identify receptors expressed in SSCs/differentiating spermatogonia. Specifically, the receptors whose expression in SSCs/differentiating spermatogonia ranks within the top 150 were selected, as expression levels below this point are generally low in these cell populations. Next, the gene expression patterns of the top 150 receptors were examined in the single-cell RNA-seq data, excluding receptors that exhibit ubiquitous expression in the testis. This filtering step helps to focus on receptors that are more specific to the target cell populations. Using the CellTalkDB database, the corresponding ligands for the selected receptors were then identified. The ligand candidates were prioritized based on their expression levels in the testes, and their known functionality.

The identified ligands were then used in the iterative process of the instant disclosure for identifying culture conditions that support testicular germ cell proliferation in vitro to refine the culture conditions. Ligands identified using this process and the concentrations of ligands that were or can be used in the iterative process are listed in Table 5. Ligands that were found to be effective to date are also noted.

TABLE 5
SSC and/or differentiating spermatogonia receptors and ligands

| Ligand | | | | |
|---|---|---|---|---|
| name | Concentration | | Effective | Receptors expressed in SPG |
| GDNF | 20-40 | ng/ml | Yes | GFRA1/GFRA2/RET/GFRA3 |
| FGF2 | 20 | ng/ml | Yes | FGFR3/FGFR1/FGFR2/FGFRL1/GPC4 |
| EGF | 20 | ng/ml | | EGFR (Note: this ligand-receptor pair did not |
| | | | | meet our threshold, however we included EGF |
| | | | | as many other groups add this factor into |
| | | | | culture media) |
| insulin | 10 | ug/ml | | INSR |
| Testosterone | 10-100 | uM | Yes | AR |
| Retinoic | 333-666 | nM | Yes | RXRA |
| Acid | | | | |
| BMF4 | 20-200 | ng/ml | | BMPR1B/BMPR1A/BMPR2 |
| BMF7 | 20-100 | ng/ml | | BMPR1B/BMPR1A/BMPR2 |
| SCF | 100-200 | ng/ml | | KITLG |
| Activin A | 20-40 | ng/ml | | ACVR2B/ACVR1B/ACVR2A |
| WNT-1 | 4-8 | ng/ml | No | RYK/FZD3/FZD7/FZD1/ROR2/LRP6 |
| WNT-2 | 100-200 | ng/ml | | FZD3/FZD7/FZD1/FZD4/LRP6 |
| WNT-3A | 20-40 | ng/ml | Yes | RYK/FZD3/FZD7/FZD1/LRP6 |
| WNT-5A | 200 | ng/ml | | RYK/FZD3/FZD7/FZD1/FZD4/LRP6/ROR2/PTK7 |
| WNT-11 | 200 | ng/ml | | FZD3/FZD7/FZD1/FZD4 |
| FGF-acidic | 2-16 | ng/ml | | FGFR3/FGFR1/FGFR2/FGFRL1 |
| (FGF1) | | | | |
| FGF9 | 5-10 | ng/ml | No | FGFR3/FGFR1/FGFR2 |
| Neurturin | 80-160 | ng/ml | Yes | GFRA1/GFRA2/RET/GFRA3 |
| (NRTN) | | | | |
| Persephin | 20-40 | ng/ml | | GFRA1/GFRA2/RET/GFRA3 |
| (PSPN) | | | | |
| Artemin | 20-40 | ng/ml | No | GFRA1/GFRA2/RET/GFRA3 |
| (ARTN) | | | | |
| Netrin-1 | 500 | ng/ml | No | NEO1 |
| (NTN1) | | | | |
| BMP1 | 110-220 | ng/ml | | BMPR1B/BMPR1A/BMPR2 |
| BMP2 | 200-400 | ng/ml | Yes | BMPR1B/BMPR1A/BMPR2 |
| BMP6 | 100-200 | ng/ml | | BMPR1B/BMPR1A/BMPR2 |
| BMP8a | 200-500 | ng/ml | | BMPR1B/BMPR1A/BMPR2 |
| BMP8b | 0.2-1 | ug/ml | No | BMPR1B/BMPR1A/BMPR2 |
| GDF6/BMP13 | 1.26-1.5 | ug/ml | No | BMPR1B/BMPR1A/BMPR2 |
| GDF9 | 250 | ng/ml | No | ACVR2A/BMPR1B/BMPR1A/BMPR2/TGFBR1 |
| GDF11/BMP11 | 5-10 | ng/ml | No | BMPR1B/BMPR1A/BMPR2/ACVR2B/ACVR1B/ACVR2A |
| inhibinA | 20-40 | ng/ml | | ACVR2B/ACVR2A |
| DHH | 200-400 | ng/ml | | PTCH1 |
| CNTN2 | 100-200 | ng/ml | No | CNTNAP2 |
| IGF1 | 10-20 | ng/ml | | IGF1R/IGF2R/INSR |
| IGF2 | 10-20 | ng/ml | | INSR/IGF1R/IGF2R |
| CFCL1 | 25-50 | ng/ml | | CNTFR/IL6ST/LIFR |
| TGFB1 | 2-4 | ng/ml | | SDC2/TGFBR1 |
| DLK1 | 0.52-1 | ug/ml | No | NOTCH1/NOTCH2 |
| DLK2 | 1-2 | ug/ml | No | NOTCH1 |
| DLL1 | 0.5-1 | ug/ml | | NOTCH1/NOTCH2 |
| DLL3 | 250-500 | ng/ml | | NOTCH1/NOTCH2 |
| NRG1-beta 1 | 0.5-20 | ng/ml | | GPC1 |
| EGF | | | | |
| Domain | | | | |
| (NRG1) | | | | |
| NRG1-beta 1 | 12.5-100 | ng/ml | | GPC1 |
| extracellular | | | | |
| domain | | | | |
| (NRG1) | | | | |
| rh beta-NGF | 2-8 | ng/ml | Yes | KIDINS220 |
| rh Midkine | 1-2 | ug/ml | Yes | GPC2 |
| Protein | | | | |
| rh HB-EGF | 1-4 | ng/ml | Yes | CD9 |
| rh Holo- | 10-100 | ug/ml | | TFRC |
| Transferrin | | | | |
| rh MIF | 80-160 | ng/ml | Yes | CXCR4/CD74 |
| rh ADAM10 | 10-20 | ng/ml | No | CADM1/TSPAN14/TSPAN15/TSPAN12 |
| rh WNT7a | 100-200 | ng/ml | | RECK |
| rh Secretin | 100-200 | nM | No | VIPR2 |
| rh LIF | 10-20 | ng/ml | Yes | IL6ST/LIFR |
| rh CXCL12 | 100-200 | ng/ml | No | CXCR4 |

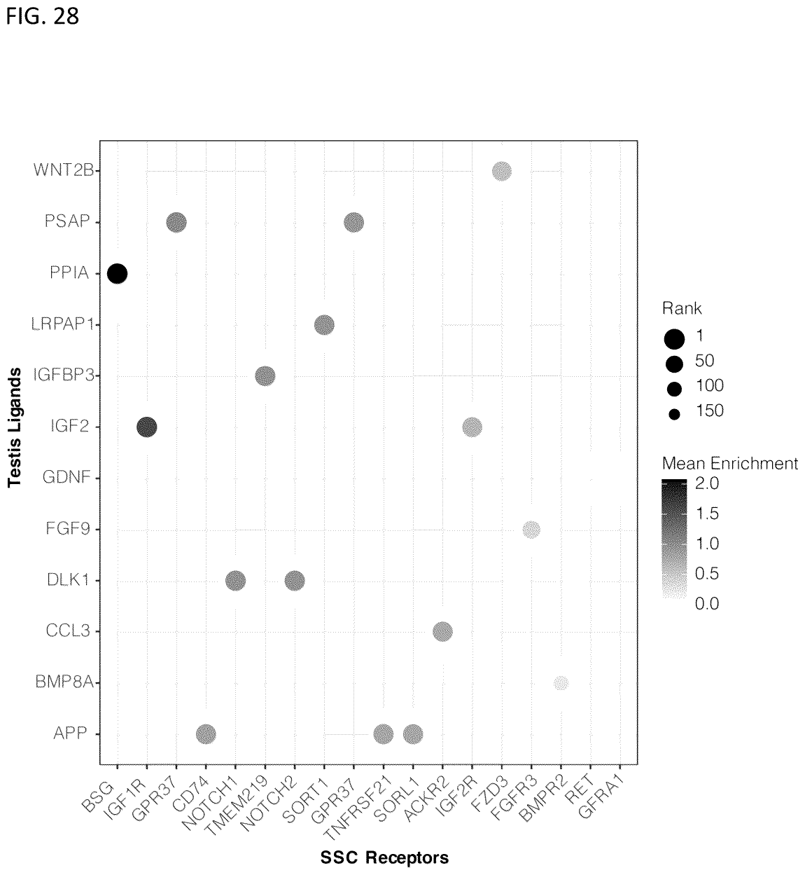

Importantly, after extensive experimentation, it was surprisingly discovered that other methods of identifying receptors and corresponding ligands such as using CellphoneDB and CellChat to analyze cell-cell interaction to find potential ligands was not successful. As shown in FIG. 28, the Cellphone DB software package fails to enrich ligand-receptor pairs of known importance to human SSC biology. The SSC receptors serving as positive controls (FGFR3, BMPR2, RET, GFRA1) showed at best modest enrichment and low ranking in the analysis with CellPhoneDB. Accordingly, it was concluded that CellPhoneDB is not well suited for uncovering biologically meaningful signaling pathways in human SSCs.

### Example 4. Improved Culture Conditions

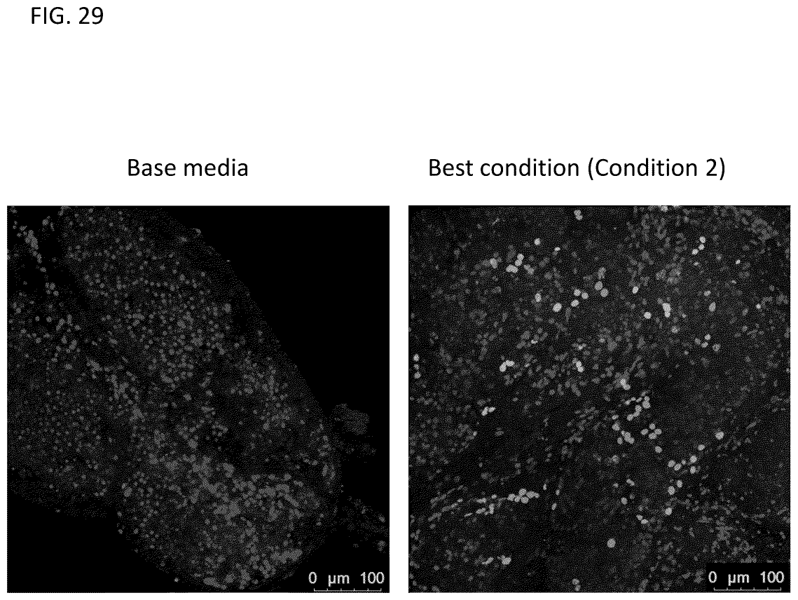

The iterative process of the instant disclosure and the process for identifying SSC ligands were used to identify a combination of factors that can extend germ cell proliferation (e.g., dish life of stem cells) to at least 21 days in cultured tubules (FIG. 29). The improved culture media is shown in Table 6 (referred to herein throughout as Control 2 media) and is referred to herein throughout as Condition 2 media.

TABLE 6
Control 2 media

| Components | Concentration | |
|---|---|---|
| αMEM | base media | |
| Knockout Serum Replacement | 10% | |
| (KSR) | | |
| Penicillin - Streptomycin | 1% | |
| GDNF | 20 | ng/ml |
| FGF2 | 20 | ng/ml |
| Insulin | 10 | ug/ml |
| EGF | 20 | ng/ml |
| Testosterone | 10 | uM |
| Echinomycin | 5 | nM |

### Example 5. Further Identification of Factors that Improve the Level of Proliferation of Testicular Germ Cells

Inhibitors of dysregulated pathways identified in Example 2 were screened using the iterative process of the instant disclosure comparing cultured to uncultured tissue to identify culture conditions that can improve the level of proliferation of germ cells.

The inhibitors used for screening were selected based on scRNA-seq results comparing cultured and uncultured somatic cells by scRNA-seq to identify dysregulated pathways (FIG. 30). In this study, the HIF-1a, apoptosis, inflammation, ROS, and angiogenesis pathways were primarily targeted to help maintain testicular somatic cell function and promote germ cell proliferation. The inhibitors used in the screen for each dysregulated pathway are shown in Tables 7-11. The level of proliferation was quantified by calculating the number of Edu+ cells (EdU stains the cells that are in the process of replication) per unit area (in μm²) in each condition and using the ratio of this parameter in the test condition to a previously identified best condition to quantify the effect of the tested condition. An example of a comparative analysis of culture conditions is shown in FIG. 31 showing the level of proliferating germ cells in tissue cultured in a first culture condition (top panels) and the level of proliferating germ cells in tissue cultured in the first culture condition further comprising nystatin (an anti-apoptosis factor) discovered to be effective at improving germ cell proliferation (bottom panels). A list of inhibitors found to be effective in this study is shown in Table 12.

TABLE 7

| Category | Inhibitor name | Concentration | |
|---|---|---|---|
| HIF inhibitor | Echinomycin | 5 | nM |
| | Amphotericin B | 0.5 | ug/ml |
| | Podofilox | 5 | nM |
| | Tanespimycin | 10 | nM |
| | 2ME2 | 1 | uM |
| | LY294002 | 10 | uM |
| | Topotecan Hydrochloride | 50 | nM |
| | Pictilisib | 50 | nM |
| | Entinostat | 200 | nM |
| | Vorinostat | 1 | uM |
| | PT-2385 | 50 | uM |
| | Acriflavine | 2 | uM |
| | VR23 | 1 | uM |
| | Carfilzomib | 20 | nM |
| | Bortezomib | 40 | nM |
| | Ganetespib | 50 | nM |
| | YC-1 | 500 | nM |
| | PX-12 | 5 | uM |
| | PI-103 | 100 | nM |
| | Torkinib | 100 | nM |
| | Epirubicin hydrochloride | 200 | nM |
| | Amentoflavone | 2 | uM |
| | SYP-5 | 10 | uM |
| | TC-S 7009 | 30 | uM |
| | PX-478 | 20 | uM |
| | Strophanthidin | 1 | uM |
| | TAS-103 HCl | 50 | nM |
| | FM19G11 | 500 | nM |

TABLE 8

| Category | Inhibitor name | Concentration | |
|---|---|---|---|
| anti- | Pifithrin-α hydrobromide | 10 | uM |
| apoptosis | | | |
| | GSK2795039 | 25 | uM |
| | Nystatin | 5 | uM |
| | Chloramphenicol | 250 | uM |
| | Tauroursodeoxycholate | 100 | uM |
| | sodium | | |
| | Rutin | 10 | uM |
| | Oxymatrine | 20 | uM |
| | DPN | 20 | nM |
| | NSC 15364 | 100 | nM |
| | BOC-D-FMK | 40 | uM |
| | Bax inhibitor peptide V5 | 20 | uM |
| | DC260126 | 5 | uM |
| | COG1410 | 10 | uM |
| | Notoginsenoside R1 | 10 | uM |
| | BI-6C9 | 10 | uM |
| | Coenzyme Q9 | 10 | uM |
| | BiP inducer X | 5 | uM |
| | Coniferaldehyde | 50 | uM |
| | Acetylshikonin | 2 | uM |
| | Asperosaponin VI | 2 | uM |
| | BRD3308 | 100 | nM |
| | Bilobalide | 3 | uM |
| | DCP-LA | 100 | nM |
| | Morroniside | 10 | uM |
| | MCL-1/BCL2-IN-2 | 5 | uM |
| | A-1331852 | 20 | nM |
| | A-1155463 | 200 | nM |

TABLE 9

| Category | Inhibitor name | Concentration | |
|---|---|---|---|
| anti- | (S)-Flurbiprofen | 1 | uM |
| inflammation | | | |
| | Benzydamine | 25 | uM |
| | hydrochloride | | |
| | RP-54745 | 5 | uM |
| | Diflucortolone valerate | 10 | uM |
| | Cortisone acetate | 300 | nM |
| | EC330 | 200 | nM |
| | SB225002 | 40 | nM |
| | Plerixafor | 20 | nM |
| | LMT-28 | 5 | uM |
| | SC144 | 1 | uM |
| | Resatorvid | 20 | nM |
| | Apilimod | 40 | nM |
| | JTE-607 | 20 | nM |
| | AX-024 HCl | 2 | nM |

TABLE 10

| Category | Inhibitor name | Concentration |
|---|---|---|
| anti- | Dimethyl-bisphenol A | 25 uM |
| angiogenesis | | |
| | Sunitinib | 50 nM |
| | Nintedanib | 50 nM |
| | Sorafenib | 50 nM |

TABLE 11

| Category | Inhibitor name | Concentration |
|---|---|---|
| ROS inhibitor | DL-a-tocopheral | 1 mM (in cell |
| | acetate (VE) | culture) |
| ROS inhibitor | L-Ascorbic acid | 1 mM (in cell |
| | 2-glucoside (VC) | culture) |
| ROS inhibitor | glutathione (GSH) | 1 mM (in cell |
| | | culture) |

TABLE 12

| | | | | | EdU+/area |
|---|---|---|---|---|---|
| | | | | | (um2) to |
| Category | inhibitor name | concentration | | quantified? | condition 2 |
| HIF inhibitor | Strophanthidin | 1 | uM | Yes | 2.2 |
| Anti-apoptosis | Notoginsenoside R1 | 10 | uM | Yes | 1.5 |
| Anti-apoptosis | Nystatin | 5 | uM | Yes | 2.1 |
| Anti-inflammation | Plerixafor | 20 | nM | Yes | 1.4 |
| Anti-inflammation | SB225002 | 40 | nM | Yes | 1.7 |

| | | | | |
|---|---|---|---|---|
| ROS inhibitor | DL-a-tocopheral | 1 | mM | tested in isolated |
| | acetate (VE) | | | tubule cell culture |
| ROS inhibitor | L-Ascorbic acid 2- | 1 | mM | tested in isolated |
| | glucoside (VC) | | | tubule cell culture |
| ROS inhibitor | glutathione (GSH) | 1 | mM | tested in isolated |
| | | | | tubule cell culture |

### Example 6. Method of Culturing Isolated Spermatogonia (SPG Culture System)

A protocol for culturing isolated spermatogonia, i.e., spermatogonia without niche cells, and independently from tubules was developed. In some aspects, tubule culture systems such as those described in Examples 2-5 can be used a learning platform to inform SPG culture.

According to the developed protocol, spermatogonia are first isolated and the isolated cells are cultured.

### Cell Isolation

Materials and Reagents.
PBS (thermoFisher scientific #1001004)
H₂O (thermoFisher scientific #10977015)
Collagenase type IV (Sigma Aldrich cat #C5138-500MG): use PBS to dilute to 10 mg/ml (10×), store at −20° C.
Trypsin-EDTA (0.25%, Invitrogen cat #25300054): stored at −20° C.
AutoMACS Running Buffer—MACS Separation Buffer (milenyl Biotec): stored at 4° C. Keep on ice during use.
FBS(Omega Scientific FB01, lot #908017): store at −20° C.
Digestion I solution (pre-warm to 37° C.): 5 ml PBS+700 ul collagenase type IV+100 ul DNase I
Digestion II solution (pre-warm to 37° C.): 500 uL Trypsin-EDTA (0.25%)+4.5 mL PBS
Strainers with mesh size 40 μm (Fisher Scientific cat #08-771-1)

Method. Spermatogonia are isolated from testicular tissue as follows. 2 g of tissue are scraped with a razor on a petri dish with razor blades to spread the tissue. The scraped tissue is added to 50 ml Eppendorf Conical Tubes with digestion I solution and shaken vigorously (a rotor at 60 rpm was used) for 5 mins. At the end of digestion, separated tubules can be detected. The tissue is then filtered with a 40 um strainer, washed with 5 mL MACS buffer, and the flow through is discarded. The tissue is then added into a new tube with 5 ml digestion II solution, and shaken vigorously (a rotor at 60 rpm was used) for 30 mins. The tissue is again filtered with a 40 um strainer, washed with 15 mL MACS buffer, and the cell suspension is transferred into a new 50 ml tube and placed on ice. 600 ul of FBS is added into the new tube to terminate the reaction and the tube is centrifuged at 300 g for 5 mins at 4° C. The supernatant is aspirated without disturbing the cell pellet, and the cells are resuspended in 1 ml MACS buffer. The cells are pelleted again and the supernatant is aspirated without disturbing the cell pellet. The cells in the cell pellet is then resuspend in 1 ml MACS buffer and filtered with a 40 um strainer. The cells in the filtrate is now ready for concentration calculation and the subsequent single-cell sequencing.

### Cell Culture

Culture media. Spermatogonia cell culture media used is as shown in Table 13 and is referred to herein throughout as C2 media. The composition of the C2 media was informed by the iterative process of the instant disclosure and/or results of the iterative process used with cultured testicular tissue.

TABLE 13
Spermatogonial culture medium

| Components | Concentration | |
|---|---|---|
| aMEM | base media | |
| Knockout Serum Replacement (KSR) | 10% | |
| Penicillin - Streptomycin | 1% | |
| GDNF | 20 | ng/ml |
| FGF2 | 20 | ng/ml |
| Insulin | 10 | ug/ml |
| EGF | 20 | ng/ml |
| Testosterone | 10 | uM |

Cells are plated at a concentration of ˜90,000 cells/cm² in petri plates and incubated for 24 hours at 35° C. After incubation, the supernatant is removed and laminin is added at a concentration of 0.25 mg/cm². The supernatant is also replated to a new plate. The media is changed every other day, at which time the top 50% of the media is removed and replaced with a similar volume of media.

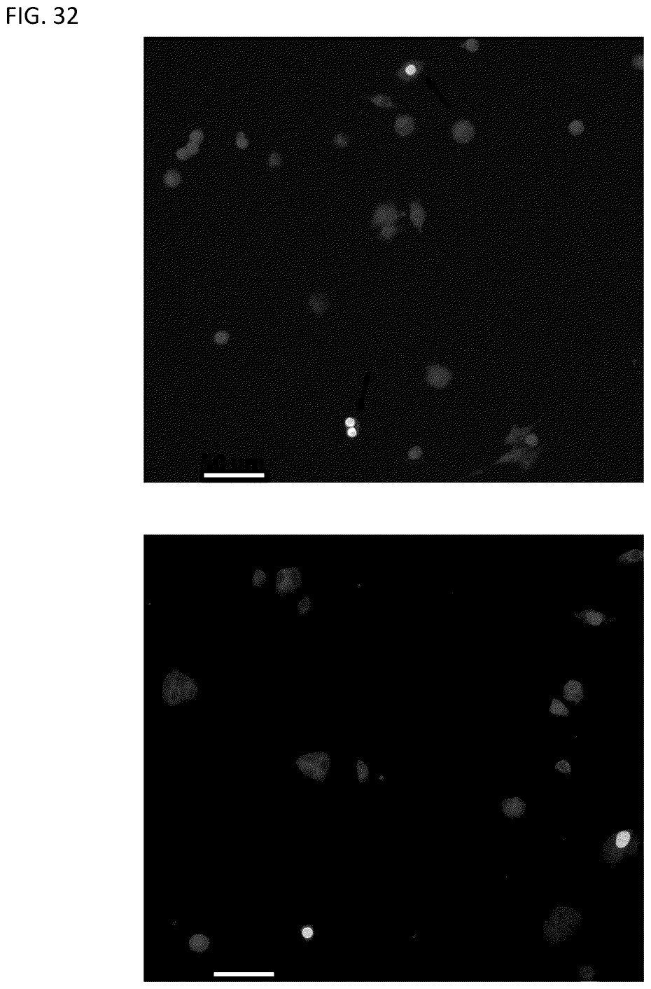

As it can be seen in FIG. 32, proliferating spermatogonia were detected at day 7 during culture and even at day 14. In fact, more proliferating spermatogonia are observed at day 14, indicating replicating spermatogonia (Table 14).

TABLE 14

| | Day 7 | Day 14 |
|---|---|---|
| DDX4 + EdU+ cells/ | 1.69% | 2.36% |
| DDX4+ cells | | |

### Example 7. Improved Method of Culturing Isolated Spermatogonia

Through the iterative process of the instant disclosure, the concentration of testosterone in C2 media (Table 13) described in Example 6 was adjusted to include eight times the levels of testosterone in C2 media improves SPG proliferation when compared to culture in C2 media as measured by the number of proliferating SPG in media after 14 days of culture.

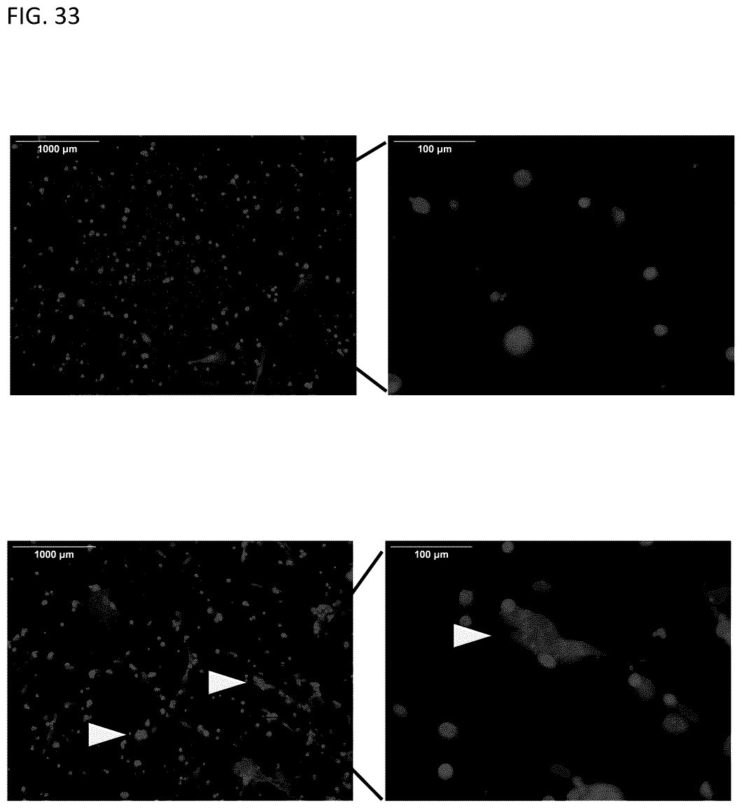

As it can be seen in FIG. 33, proliferating spermatogonia were detected at days 14 during culture in C2 media, and clusters of proliferating germ cells during culture in C2 media.

### Example 8. A Hybrid System for In Vitro Culture of Spermatogonia

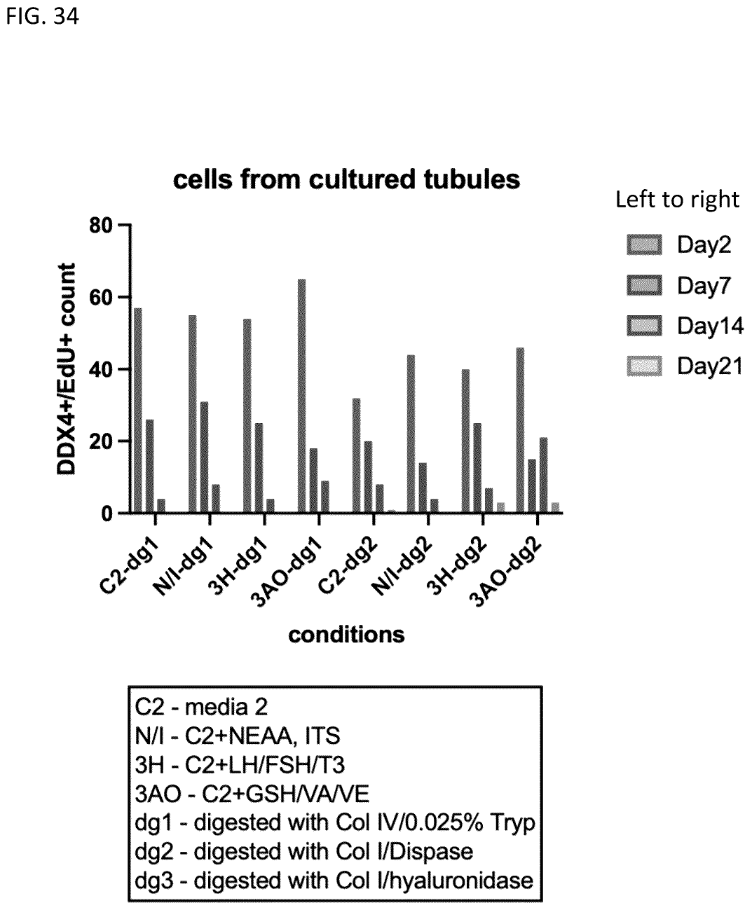

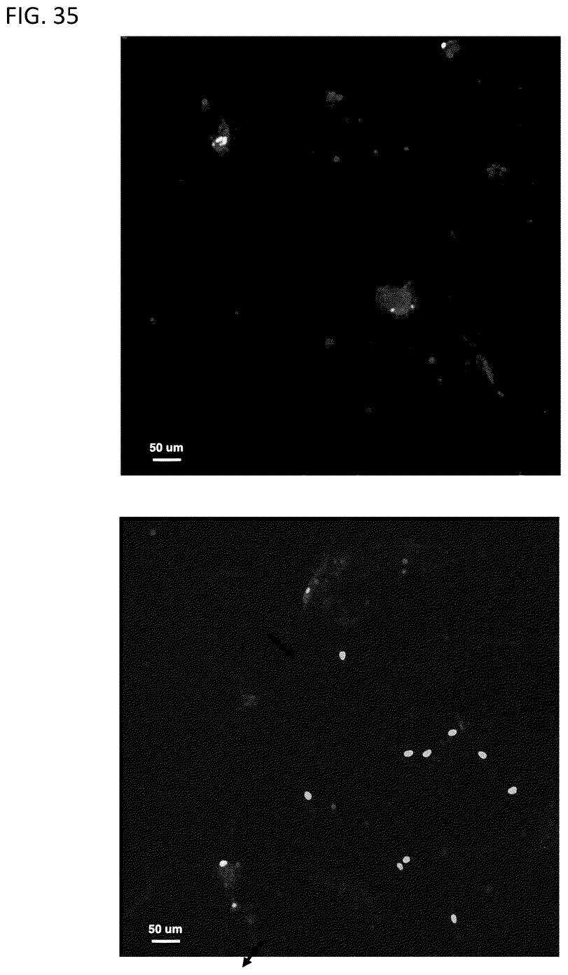

After extensive experimentation, it was discovered that using a hybrid system comprising a combination of the testicular tissue culture system of Example 2 using the control 2 culture media SPG culture system of using the C2 medium, was able to significantly increase the length time germ cells can proliferate in culture. More specifically, it was discovered that by culturing testicular tissue using the tissue culture system of Example 2 in the control 2 culture media followed by culturing SPG using the SPG culture system of Example 7, the germ cells were able to continue proliferating in culture for at least 21 days (FIGS. 34 and 35). More detailed descriptions of methods used are described further below.

As shown in FIGS. 34 and 35, SPG were capable of proliferating at least 21 days after a 7-day culture period in tubules. Various cell preparation and isolation methods and various culture conditions were tested. Methods of cell preparation and culture conditions tested are as described in Table 15. Importantly, it was found that the method used to isolate tissues and cells and the culture media are both important for successful in extending proliferative life of SPG in culture. More specifically, the cell preparation and culture conditions most effective at increasing proliferative life of SPG are as follows: the tubules are cultured in C2 media; the SPG are isolated by COL I+Dispase, the plate is coated with laminin, the cells are cultured in media C2+3OA (3OA is a combination of 3 anti-oxidant molecules, including GSH, VC and VA).

TABLE 15

| Medium | | Cell prep | |
|---|---|---|---|
| C2 | C2 media alone | Dg1 | Digested with Col IV |
| | | | and 0.025% Tryp |
| N/I | C2 media supplemented | Dg2 | Digested with Col IV |
| | with NEAA and ITS | | and Dispase |
| 3H | C2 media supplemented | Dg3 | Digested with Col IV |
| | with LH, FSH, and T3 | | and Hyaluronidase |
| 3AO | C2 media supplemented | | |
| | with GSH, VA, and VE | | |

### Samples.

Samples were received from Donor Connect. The samples were processed and fertile samples were chosen to set up the experiment. The tissue was cut into small pieces of an approximate size of 0.5 cm×0.5 cm. The pieces were cultured in two 6 well cell culture plates with three pieces in a well. Media used for culture was condition 2 media with. The tissue pieces were incubated at 35° C. with 5% CO2 for 7 days with media change done every alternative day. On day 7 of the culture, all the tissue pieces were pooled and cells were isolated from them. Two different SPG cell isolation methods were followed.

### Method I

### Materials and Reagents

PBS (thermoFisher scientific #1001004)
Ultrapure distilled water (thermoFisher scientific #10977015)
collagenase type IV (Sigma Aldrich cat #C5138-500MG): use PBS to dilute to 10 mg/ml (10×), store at −20° C.
Trypsin-EDTA (0.25%, Invitrogen cat #25300054): store at −20° C.
autoMACS Running Buffer—MACS Separation Buffer (Milenyl Biotec): store at 4° C. Put on ice during use.
FBS(Omega Scientific FB01, lot #908017): store at −20° C.
Digestion I solution (pre-warm to 37° C.): 5 ml PBS+700 ul collagenase type IV
Digestion II solution (pre-warm to 37° C.): 500 ul Trypsin-EDTA (0.25%)+4.5 ml PBS.
Cell strainers with mesh size 40 μm (Fisher Scientific cat #08-771-2)

### Procedure

Two g of tissue are added to a petri dish and scraped with razor blades to spread the tissue. The scraped tissue is transferred to 50 ml Eppendorf Conical Tubes with digestion I solution, incubated at 37° C., and shaken vigorously (a rotor at 60 rpm was used) for 3 mins. At the end of digestion, separated tubules can be detected. The tissue is then filtered with a 40 μm strainer and the flow through is discarded. The tissue is then added into a new 50 ml tube with digestion solution II, incubated at 37° C., and shaken vigorously about every 5 min (a rotor at 60 rpm can be used) for 30 mins. At the end of digestion, the wall of tubules is blurred. The tissue is again filtered with a 40 μm strainer and the wash and the cell suspension is transferred into a new 50 ml tube and placed on ice. 600 ul of FBS is added into the new tube to terminate the reaction and the tube is centrifuged at 300 g for 5 mins at 4° C. The supernatant is aspirated without disturbing the cell pellet, and the cells are resuspended in 1 ml MACS buffer. The cells are pelleted again in a 1.5 ml tube for 5 min at 300 g and the supernatant is aspirated without disturbing the cell pellet. The cells in the cell pellet is then resuspend in C2 media. The cells are counted and plated per the required cell density per well.

### Method II

### Materials and Reagents

Enzyme stocks: 50× Collagenase type I (thermo 17100017) in HBSS (10 U/ul), −20° C. in TC, 100× Dispase in DPBS (240 U/ml), −20° C. in TC
Digestion I: 5 ml of 200 U/ml Collagenase type I (1×) in HBSS
Digestion II: 5 ml of 200 U/ml Collagenase type I (1×), 2.4 U/ml Dispase (1×), 1 mM CaCl₂) in HBSS

### Procedure

Two g of tissue are added to a petri dish and scraped with razor blades to spread the tissue. The scraped tissue is transferred to 50 ml Eppendorf Conical Tubes with digestion I solution, incubated at 37° C., and shaken vigorously (a rotor at 60 rpm was used) every 1 min for 3-5 mins. At the end of digestion, separated tubules can be detected. The tubules are filtered with a 40 μm strainer, rinsed with ˜5 ml cold MACS buffer, and tissue on top of the strainer is collected into a tube with 5 ml Digestion II buffer. Discard the cells passed through. The tissue is digested in Digestion II at 37° C. in a water bath for 30 min, with vigorous shaking every 5 mins. At the end of digestion, the wall of tubules should be blurred. The tissue from the previous step is filtered through a 40 μm strainer. The cell suspension is added into a new 50 ml tube (on ice) and 600 ul FBS as well as 100 ul DNase I are added into the new tube to terminate the reaction, add 15 ml cold MACS buffer and centrifuged at 300 g for 5 mins at 4° C. The supernatant is aspirated without disturbing the cell pellet and the cells are resuspended in 1 ml MACS buffer, transferred to 1.5 ml tube, spun at 300 g for 5 min and resuspended in C2 media. The cells are counted.

### Cell Culture

The isolated cells were cultured in 96 well cell culture plates with a seeding density of 30,000 live cells per well in 200 ul media. The media tested were as in Table 15. MatriClone at 0.16 ul was added to each well. The cell culture plates were incubated at 35° C. with 5% CO2. Media changes were done every other day by aspirating 100 ul of old media and supplementing the same amount of fresh media. Four different plates were set-up to stain and observe the cells at day 2, day 7, day 14 and day 21. The cells were stained with EdU and DDX4. EdU stains the cells that are in the process of replication and DDX4 stains germ cells.

Method for Staining
1. Label cells with EdU
   a. In cell culture/assay plate, change media and add 10 mM EdU DMSO stock (2000×) directly in media to make 5 uM working concentration.
   b. Incubate for 24 h or 48 h in TC incubator depending on assay needs.
2. Fix cells
   a. After incubation, aspirate media and rinse once with PBS
   b. Fix cells by adding 4% paraformaldehyde in PBS and incubate at RT for 10-15 min.
   c. Rinse cells 3×5 min with PBS at RT
3. Permeabilize cells
   a. Prepare permeabilization buffer by adding 0.1% TritonX-100 in PBS
   b. Incubate cells with permeabilization buffer at RT for 10 min
   c. Rinse cells 3×5 min with PBS at RT
4. Blocking (SuperBlock solution from Thermo)
   a. Incubate cells in blocking buffer for 1 h at RT
5. Primary antibody
   a. Prepare primary working solution by adding DDX4 1:400 in primary antibody buffer
   b. Incubate cells with primary antibody solution for 1 h at RT or overnight at 4° C.
   c. Rinse cells 3×5 min with PBS at RT
6. Secondary antibody
   a. Prepare secondary antibody solution by adding fluorophore conjugated secondary antibody 1:800 in secondary antibody buffer, minimize light exposure
   b. Incubate cells with 2nd antibody for 1 h at RT in the dark
   c. Rinse cells 3×5 min with PBS at RT in the dark
7. EdU detection
   a. Make EdU reaction cocktail according to the Click-iT EdU labeling kit (500 ul reaction mixture: 438 ul 1× click it reaction buffer+10 ul CuSO4+2.5 ul Fluorescent Azide+50 ul 1× reaction buffer additive. Note: protect from light and use within 15 mins.)
   b. Apply to cells and incubate for 30 min at RT
   c. Rinse cells 3×5 min with PBS at RT in the dark
8. Stain the nuclei
   a. Prepare hoechst dilution 1:10,000 in PBS
   b. Incubate cells with the hoechst solution for 5 min at RT in the dark
   c. Rinse cells 3×5 min with PBS at RT in the dark
9. Mount* (if observe immediately, just leave cells in PBS)
   a. Add ProLong Gold mountant to mount cells and cover with a coverslip for imaging.

The plates were observed under the microscope with three different channels of light.

What is claimed is:

1. An iterative process for identifying culture conditions supportive of testicular germ cell proliferation in vitro, the process comprising:
a. identifying one or more dysregulated pathways in testicular cells cultured in a first set of culture conditions by:
i. culturing testicular tissue in vitro in a first culture medium, wherein the testicular tissue comprises seminiferous tubules and testicular germ cells, and wherein the first culture medium supports a first level of proliferation of germ cells;
ii. profiling transcriptomes of single testicular cells obtained from the testicular tissue using single cell RNA sequencing (scRNA-seq);
iii. assigning a cell type to each single testicular cell using cell type-specific gene markers expressed in each cell;
iv. identifying RNA transcripts differentially expressed in each cell type when compared to RNA transcripts expressed in cells of corresponding cell types obtained from control tissue, wherein differentially expressed RNA transcripts identify one or more dysregulated biological pathways in the testicular cell types cultured in the first set of culture conditions;
b. identifying one or more factors that improve the level of proliferation of testicular germ cells by:
i. culturing testicular tissue in vitro in one or more second culture media, wherein the one or more second culture media comprise the first culture medium supplemented with one or more factors that regulate a biological pathway identified in (a);
ii. identifying one or more second culture media that support improved levels of germ cell proliferation when compared to the first level of germ cell proliferation in the first culture medium, thereby identifying the one or more factors that improve the level of proliferation of testicular germ cells;
c. iteratively repeating steps (a) and (b) to identify additional factors or to identify combinations of factors identified in step (b) that improve the level of proliferation of testicular germ cells in vitro;
wherein culture conditions supportive of testicular germ cell proliferation in vitro comprise one or more factors identified in steps (b) and (c).

2. The process of claim 1, wherein the first culture medium is base medium comprising αMEM and 10% KSR.

3. The iterative process of claim 1, wherein the first culture medium is base medium comprising αMEM and 10% KSR supplemented with factors or combinations of factors identified in a previous round of the iterative process.

4. The iterative process of claim 1, wherein testicular germ cell proliferation comprises proper identity, growth, development, survival, and replication of the testicular germ cells in vitro.

5. The iterative process of claim 1, wherein cells of testicular tissue cultured in the culture media that support testicular germ cell proliferation comprise an expressed RNA transcript profile substantially similar to the expressed RNA transcript profile of cells of testicular tissue directly isolated from testis of adult males.

6. The iterative process of claim 1, wherein cells of testicular tissue cultured in culture media that support testicular germ cell proliferation comprise no dysregulated pathways.

7. The process of claim 1, wherein control tissue is directly isolated from a subject.

8. The process of claim 1, wherein control tissue is previously cultured in a discovered medium.

9. The iterative process of claim 1, wherein the testicular tissue is obtained from a healthy adult subject, an infertile or sub-fertile adult subject, or a pre-pubertal subject.

10. The iterative process of claim 1, wherein the testicular tissue is directly isolated from testis of male subjects.

11. The iterative process of claim 1, wherein one or more second culture media that support testicular germ cell proliferation comprise base culture media supplemented with the one or more factors that improve the level of proliferation of testicular germ cells.

12. The iterative process of claim 1, wherein the one or more dysregulated pathways comprise one or more pathways of apoptosis.

13. The iterative process of claim 1, wherein the one or more dysregulated pathways comprise one or more pathways of hypoxia-inducible factor (HIF).

14. The iterative process of claim 13, wherein culture media that support testicular germ cell proliferation comprise a hypoxia-inducible factor (HIF) inhibitor, a gonadocorticoid, a fibroblast growth factor receptor (FGFR) protein ligand, or any combination thereof.

15. The iterative process of claim 14, wherein the HIF inhibitor is echinomycin and wherein one or more second culture media that support testicular germ cell proliferation comprise echinomycin at a concentration ranging from 2 nM to 7 nM.

16. The iterative process of claim 14, wherein the gonadocorticoid is testosterone and GDNF, wherein one or more second culture media that support testicular germ cell proliferation comprise testosterone at a concentration ranging from 1.5×10⁻⁶M to 0.5×10⁻⁸M and GDNF at a concentration ranging from 7 ng/mL to 12 ng/mL.

17. The iterative process of claim 14, wherein the FGFR protein ligand is bFGF (FGF2) and wherein one or more second culture media that support testicular germ cell proliferation comprise bFGF at a concentration ranging from 7 ng/mL to 12 ng/mL.

18. The iterative process of claim 1, wherein the culture conditions supportive of testicular germ cell proliferation in vitro comprise base culture media supplemented with echinomycin, testosterone, GDNF, and bFGF.

19. The iterative process of claim 18, wherein the culture conditions supportive of testicular germ cell proliferation in vitro comprise base culture media comprising αMEM and 10% KSR supplemented with 2 nM to 7 nM echinomycin, 1.5×10⁻⁶M to 0.5×10⁻⁸M, testosterone, 7 ng/mL to 12 ng/mL GDNF, and 7 ng/mL to 12 ng/mL bFGF.
